(19) United States
(12) Patent Application Publication
(10) Pub. No.: US 2019/0169218 A1
(43) Pub. Date: Jun. 6, 2019

(54) AMPHIPATHIC COMPOUND HAVING NOVEL PENTA-SACCHARIDE HYDROPHILIC GROUP AND USE THEREOF

(71) Applicant: Industry-University Cooperation Foundation Hanyang University Erica Campus, Ansan-si, Gyeonggi-do (KR)

(72) Inventors: Pil Seok CHAE, Ansan-si, Gyeonggi-do (KR); Muhammad EHSAN, Ansan-Si, Gyeonggi-Do (KR)

(21) Appl. No.: 16/092,938

(22) PCT Filed: Apr. 14, 2017
(86) PCT No.: PCT/KR2017/004066

§ 371 Date: Oct. 11, 2018

(30) Foreign Application Priority Data
Apr. 14, 2016 (KR) 10-2016-0045395

(51) Int. Cl. C07H 15/04; G01N 33/68

(52) U.S. Cl. CPC C07H 15/04; C07K 1/145; G01N 33/68

(57) ABSTRACT

Disclosed are an amphipathic compound having a penta-saccharide hydrophilic group, a method of preparing the same, and a method of extracting, solubilizing, stabilizing, crystallizing or analyzing membrane proteins and membrane protein complexes using the same. In particular, since the compound has a high-density penta-saccharide hydrophilic group composed of five glucose units, the compound may have an excellent effect on crystallization of membrane proteins. In addition, since the hydrophilic group used in the amphipathic compound has a novel structure, the hydrophilic group may be applied to the development of various amphipathic molecules.

### CROSS-REFERENCE TO RELATED APPLICATION

This application claims priority to and the benefit of Korean Patent Application No. 200X-XXXXX filed on XXX X, 200X, the disclosure of which is incorporated herein by reference in its entirety.

### BACKGROUND

### 1. Field of the Invention

The present invention relates to an amphipathic compound having a newly developed penta-saccharide hydrophilic group and a method of preparing the same, and more particularly, to a method of extracting, solubilizing, stabilizing, crystallizing or analyzing membrane proteins using the same.

### 2. Discussion of Related Art

Membrane proteins play an important role in biological systems. Since these bio-macromolecules (i.e., membrane proteins) contain hydrophilic and hydrophobic domains, amphipathic molecules are needed to extract membrane proteins from lipid bilayers and to solubilize and stabilize the same in aqueous solutions.

To analyze the structures of membrane proteins, it is necessary to obtain high quality membrane protein crystals. For this purpose, structural stability of the membrane proteins in an aqueous solution should be preferentially achieved. Although the number of existing amphipathic molecules that have been used in membrane protein studies is more than 100, only 5 thereof have been actively used for membrane protein structure studies. These five amphipathic molecules include n-octyl-β-D-glucopyranoside (OG), n-nonyl-β-D-glucopyranoside (NG), n-decyl-β-D-maltopyranoside (DM), n-dodecyl-β-D-maltopyranoside (DDM), and lauryldimethylamine-N-oxide (LDAO) (Non-Patent Documents 1 and 2). However, since various membrane proteins surrounded by these molecules are easily denatured or aggregate and quickly lose functions thereof, there are considerable limitations in studying the functions and structures of membrane proteins using these molecules. This is because the chemical structure of conventional molecules is so simple that the molecules cannot exhibit sufficiently diverse properties.

In particular, for membrane protein crystallization, it is important to form small complexes with excellent capacity to stabilize membrane proteins. Most conventional materials do not have these two properties at the same time. Since current tools have limitations in analyzing membrane protein structure and future research will address less structurally stable membrane proteins, amphipathic molecules having various desirable properties, such as excellent membrane protein stabilization and small complex formation, are required.

Accordingly, the present inventors developed novel amphipathic compounds having a glucose-centered high-density hydrophilic group, and completed the present invention by confirming that the compound is excellent in solubilizing, stabilizing and crystallizing membrane proteins.

### NON-PATENT DOCUMENTS

(Non-Patent Document 1) S. Newstead et al., Protein Sci. 17 (2008) 466-472.

(Non-Patent Document 2) S. Newstead et al., Mol. Membr. Biol. 25 (2008) 631-638.

### SUMMARY OF THE INVENTION

Therefore, the present invention has been made in view of the above problems, and it is an objective of the present invention to provide a compound represented by Formula 1.

It is another objective of the present invention to provide a composition for extracting, solubilizing, stabilizing, crystallizing or analyzing membrane proteins including the compound.

It is still another objective of the present invention to provide a method of preparing the compound.

It is yet another objective of the present invention to provide a method of extracting, solubilizing, stabilizing, crystallizing or analyzing membrane proteins using the compound.

In accordance with the present invention, the above and other objectives can be accomplished by the provision of a compound represented by Formula 1 below:

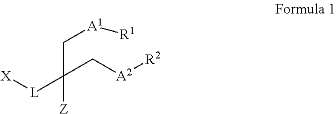

wherein L may represent a methylene group or a direct bond;

Each of A¹ and A² may represent a methylene group or an oxygen atom;

Each of R¹ and R² may independently represent a substituted or unsubstituted C₃-C₂₀ alkyl group, a substituted or unsubstituted C₃-C₂₀ cycloalkyl group, or a substituted or unsubstituted C₃-C₂₀ aryl group;

X may represent a glucose-centered branched penta-saccharide linked by oxygen; and

Z may represent a hydrogen atom or —CH₂-A³-R³, wherein A³ may represent a methylene group or an oxygen atom, and R³ may independently represent a substituted or unsubstituted C₃-C₂₀ alkyl group, a substituted or unsubstituted C₃-C₂₀ cycloalkyl group, or a substituted or unsubstituted C₃-C₂₀ aryl group.

The compound according to the above embodiment may have a penta-saccharide as a hydrophilic group.

As used herein, the term “saccharide” refers to a compound having a relatively small molecular size among carbohydrates and having a sweet taste when dissolved in water. Saccharides are classified into monosaccharides, disaccharides, and polysaccharides depending on the number of molecules constituting a sugar.

A saccharide used in the above embodiment may be a penta-saccharide composed of a total of five glucose units, in which one glucose unit is positioned at the center of a hydrophilic group and four glucose units are radially connected thereto. Each of the four glucose units may be directly connected to the central glucose unit via a glycosidic bond or may be connected to each other via an alkylene spacer.

Thus, the hydrophilic group of the compound was not previously used, has a high hydrophilic density, and may be structurally distinguished from existing amphipathic compounds. In addition, since the five saccharides are densely interconnected, an increase in the length of the hydrophilic group may be minimized while increasing the size of the hydrophilic group. As a result, the size of the complex may be reduced when membrane proteins and the compound are complexed. When the complex of the compound and the membrane proteins is small, high quality membrane protein crystals may be obtained (G. G. Prive, Methods 2007, 41, 388-397). In particular, amphipathic molecules having a small hydrophilic group such as a glucoside may have an excellent effect on the crystallization of membrane proteins.

In addition, when Z represents hydrogen, R¹ and R² may act as hydrophobic groups, or when Z represents —CH₂-A³-R³, R¹ to R³ may act as hydrophobic groups. As hydrophobic groups, two or three alkyl groups were introduced to the compound according to one embodiment of the present invention to optimize a hydrophilic-lipophilic balance.

In the compound according to one embodiment of the present invention, hydrophobic groups and hydrophilic groups may be linked via alkyl or ether linkers. Specifically, according to embodiments of the present invention, when Z represents hydrogen, A₁ and A₂ may have an alkyl linker having a methylene group (—CH₂—) or A₁ and A₂ may have an ether (—O—) linker having an oxygen atom (O). In addition, when Z represents —CH₂-A³-R³, one or more of A¹ to A³ may independently represent a methylene group (—CH₂—) or an oxygen atom.

Specifically, L may represent a methylene group; A¹ and A² may represent methylene groups; each of R¹ and R² may independently represent a substituted or unsubstituted C₃-C₂₀ alkyl group, a substituted or unsubstituted C₃-C₂₀ cycloalkyl group, or a substituted or unsubstituted C₃-C₂₀ aryl group; and Z may represent a hydrogen atom. More specifically, L may represent a methylene group; A¹ and A² may represent methylene groups; R¹ and R₂ may represent substituted or unsubstituted C₅-C₁₅ alkyl groups; R¹ and R² may be the same; and Z may represent a hydrogen atom.

In particular, L may represent a direct bond; A¹ and A² may represent oxygen atoms; each of R¹ and R² may independently represent a substituted or unsubstituted C₃-C₂₀ alkyl group, a substituted or unsubstituted C₃-C₂₀ cycloalkyl group, or a substituted or unsubstituted C₃-C₂₀ aryl group; and Z may represent a hydrogen atom. More specifically, L may represent a direct bond; A¹ and A² may represent oxygen atoms; R¹ and R² may represent substituted or unsubstituted C₅-C₁₅ alkyl groups; R¹ and R² may be the same; and Z may represent a hydrogen atom.

In particular, L may represent a methylene group; Z may represent —CH₂-A³-R³, wherein one or more of A¹ to A³ may be oxygen atoms and the other(s) may be a methylene group; each of R¹ to R³ may independently represent a substituted or unsubstituted C₃-C₂₀ alkyl group, a substituted or unsubstituted C₃-C₂₀ cycloalkyl group, or a substituted or unsubstituted C₃-C₂₀ aryl group; and R¹ to R³ may be the same. More specifically, L may represent a methylene group; Z may represent —CH₂-A³-R³, wherein one or more of A¹ to A³ may be oxygen atoms and the other(s) may be a methylene group; each of R¹ to R³ may independently represent a substituted or unsubstituted C₃-C₂₀ alkyl group; and R¹ to R³ may be the same.

In one embodiment of the present invention, the expression “alkyl-based penta-saccharide amphiphiles (PSAs)” refers to a compound, wherein L represents a methylene group; A₁ and A₂ represent methylene groups; and Z represents a hydrogen atom.

In another embodiment of the present invention, the expression “ether-based penta-saccharide amphiphiles (PSEs)” refers to a compound, wherein L represents a direct bond; A₁ and A₂ represent oxygen atoms; and Z represents a hydrogen atom.

In another embodiment of the present invention, the expression “tripod penta-saccharide amphiphiles (TPSs)” refers to a compound, wherein L represents a methylene group; and Z represents —CH₂-A³-R³, wherein one or more of A¹ to A³ represent oxygen atoms.

The compound may correspond to one of Formulas 2 to 18 according to one embodiment of the present invention, without being limited thereto.

In one embodiment of the present invention, the expression “PSA-C9” refers to a compound, wherein L represents a methylene group; A₁ and A₂ represent methylene groups; R¹ and R² represent unsubstituted C₇ alkyl groups; and Z represents a hydrogen atom. Therefore, the compound may be represented by Formula 2 below;

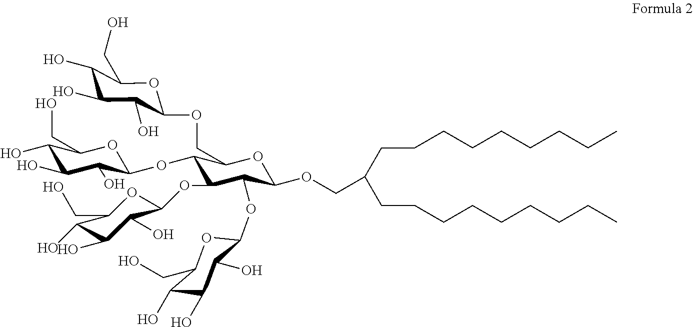

In another embodiment of the present invention, the expression “PSA-C10” refers to a compound, wherein L represents a methylene group; A¹ and A² represent methylene groups; R¹ and R² represent unsubstituted C₈ alkyl groups; and Z represents a hydrogen atom. Therefore, the compound may be represented by Formula 3 below:

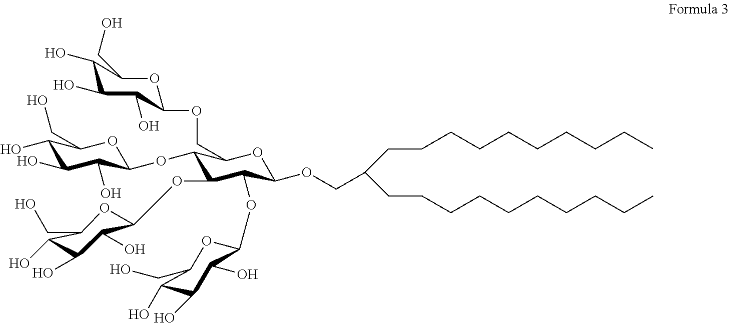

In another embodiment of the present invention, the expression “PSA-C11” refers to a compound, wherein L represents a methylene group; A¹ and A² represent methylene groups; R¹ and R² represent unsubstituted C₉ alkyl groups; and Z represents a hydrogen atom. Therefore, the compound may be represented by Formula 4 below:

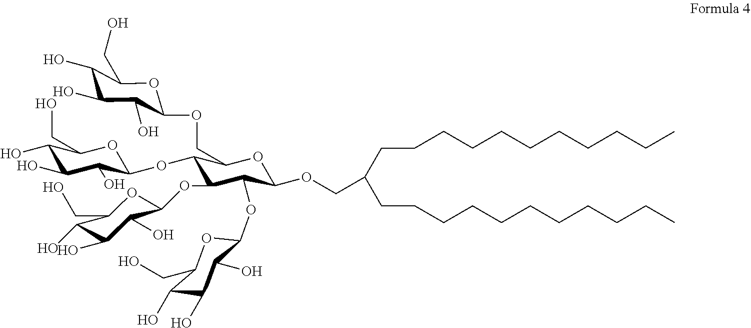

In another embodiment of the present invention, the expression “PSE-C7” refers to a compound, wherein L represents a direct bond; A¹ and A² represent oxygen atoms; R¹ and R² represent unsubstituted C₇ alkyl groups; and Z represents a hydrogen atom. Therefore, the compound may be represented by Formula 5 below:

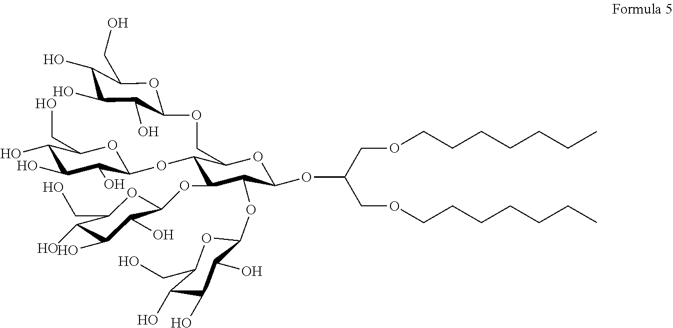

In another embodiment of the present invention, the expression “PSE-C9” refers to a compound, wherein L represents a direct bond; A¹ and A² represent oxygen atoms; R¹ and R² represent unsubstituted C₉ alkyl groups; and Z represents a hydrogen atom. Therefore, the compound may be represented by Formula 6 below:

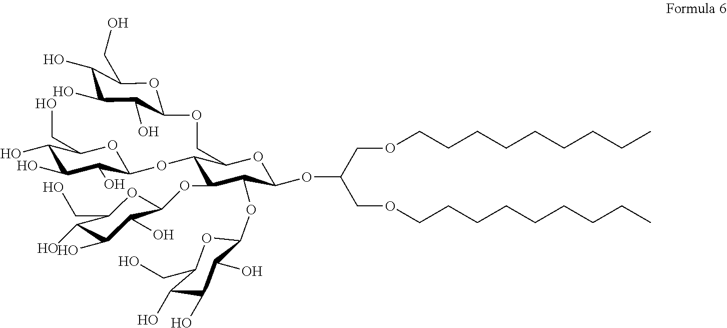

In another embodiment of the present invention, the expression “PSE-C11” refers to a compound, wherein L represents a direct bond; A¹ and A² represent oxygen atoms; R¹ and R²represent unsubstituted C₁₁ alkyl groups; and Z represents a hydrogen atom. Therefore, the compound may be represented by Formula 7 below:

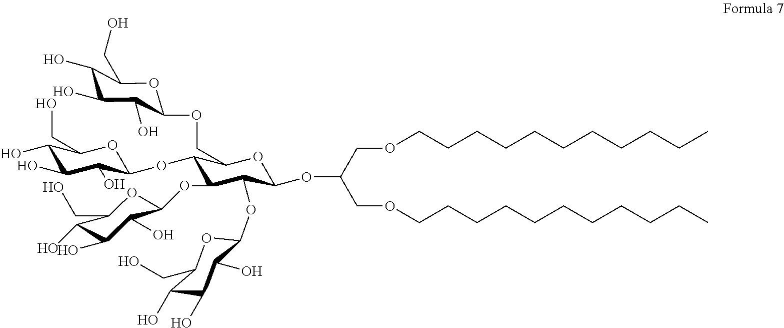

In another embodiment of the present invention, the expression “PSE-C13” refers to a compound, wherein L represents a direct bond; A¹ and A² are oxygen atoms; R¹ and R² are unsubstituted C₁₃ alkyl groups; and Z represents a hydrogen atom. Therefore, the compound may be represented by Formula 8 below:

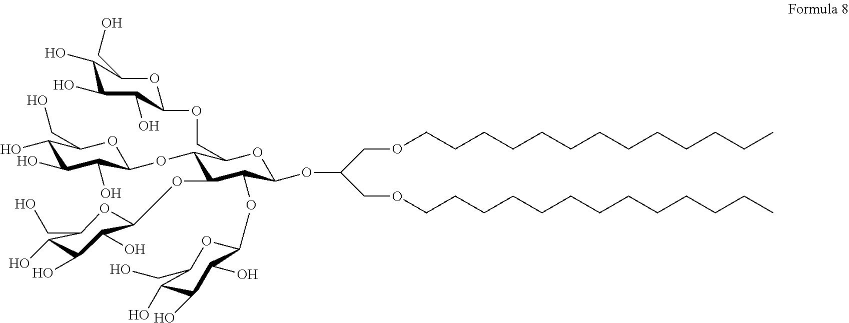

In another embodiment of the present invention, the expression “TPS-E6” refers to a compound, wherein L represents a methylene group; and Z represents —CH₂-A³-R³, wherein A¹ to A³ represent oxygen atoms and R¹ to R³ represent unsubstituted C₆ alkyl groups. Therefore, the compound may be represented by Formula 9 below:

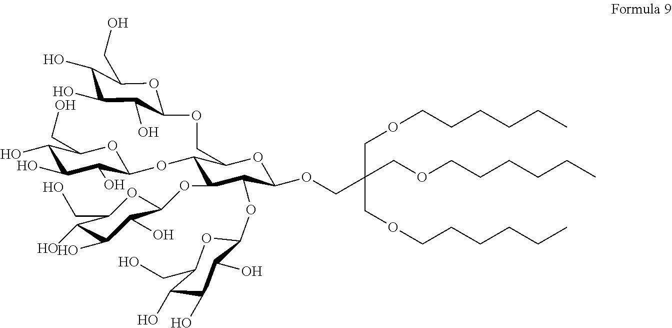

In another embodiment of the present invention, the expression “TPS-E7” refers to a compound, wherein L represents a methylene group; and Z represents —CH₂-A³-R³, wherein A¹ to A³ represent oxygen atoms and R¹ to R³ represent unsubstituted C₇ alkyl groups. Therefore, the compound may be represented by Formula 10 below:

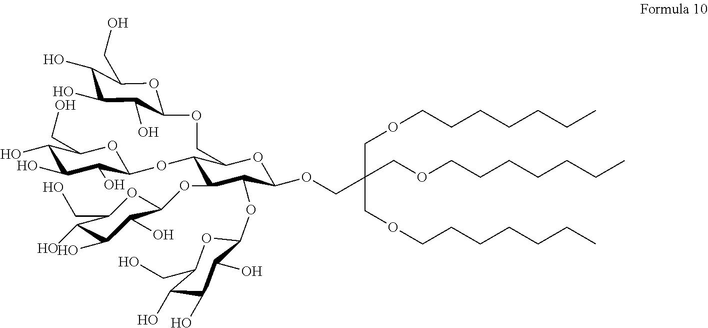

In another embodiment of the present invention, the expression “TPS-E8” refers to a compound, wherein L represents a methylene group; and Z represents —CH₂-A³-R³, wherein A¹ to A³ represent oxygen atoms and R¹ to R³ represent unsubstituted C₆ alkyl groups. Therefore, the compound may be represented by Formula 11 below:

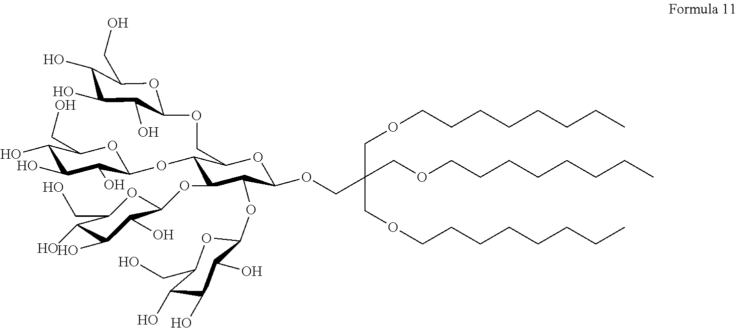

In another embodiment of the present invention, the expression “TPS-A6” refers to a compound, wherein L represents a methylene group; and Z represents —CH₂-A³-R³, wherein A¹ and A³ represent methylene groups, A² represents an oxygen atom, and R¹ to R³ represent unsubstituted C₆ alkyl groups. Therefore, the compound may be represented by Formula 12 below:

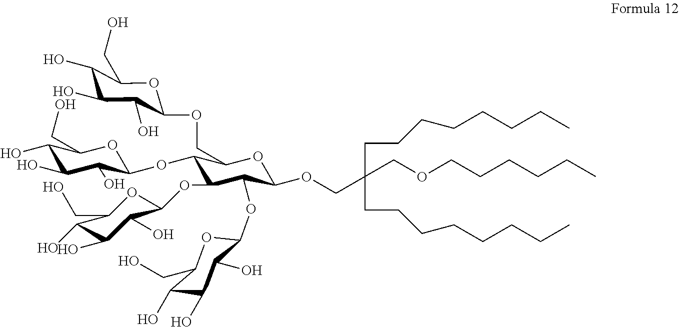

In another embodiment of the present invention, the expression “TPS-A7” refers to a compound, wherein L represents a methylene group; and Z represents —CH₂-A³-R³, wherein A¹ and A³ represent methylene groups, A² represents an oxygen atom, and R¹ to R³ represent unsubstituted C₇ alkyl groups. Therefore, the compound may be represented by Formula 13 below:

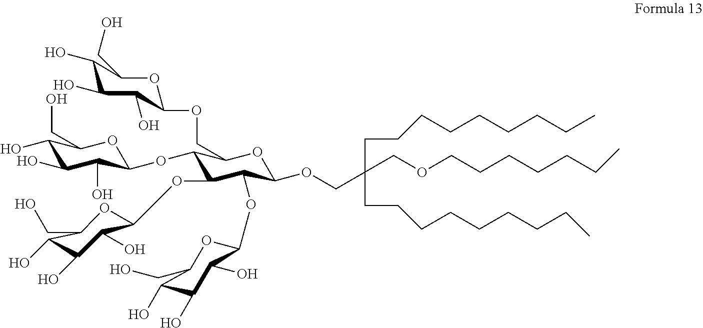

In another embodiment of the present invention, the expression “TPS-A8” refers to a compound, wherein L represents a methylene group; and Z represents —CH₂-A³-R³, wherein A¹ and A³ represent methylene groups, A² represents an oxygen atom, and R¹ to R³ represent unsubstituted C₈ alkyl groups. Therefore, the compound may be represented by Formula 14 below:

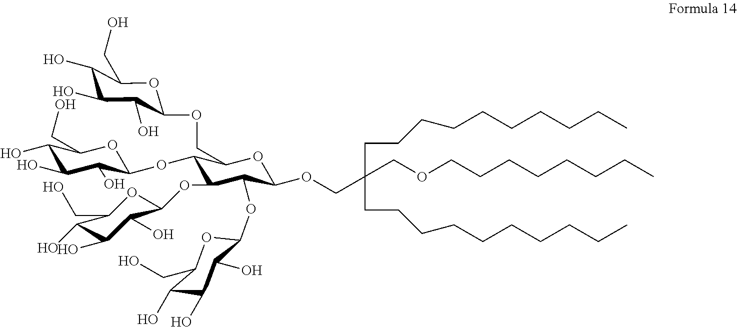

In another embodiment of the present invention, the expression “TPS-E8L” refers to a compound, wherein L represents a methylene group; Z represents —CH₂-A³-R³, wherein A¹ to A³ represent oxygen atoms and R¹ to R³ represent unsubstituted C₈ alkyl groups; and X represents a penta-saccharide, in which each of four glucose units is linked to a central glucose core via a propylene spacer. Therefore, the compound may be represented by Formula 15 below:

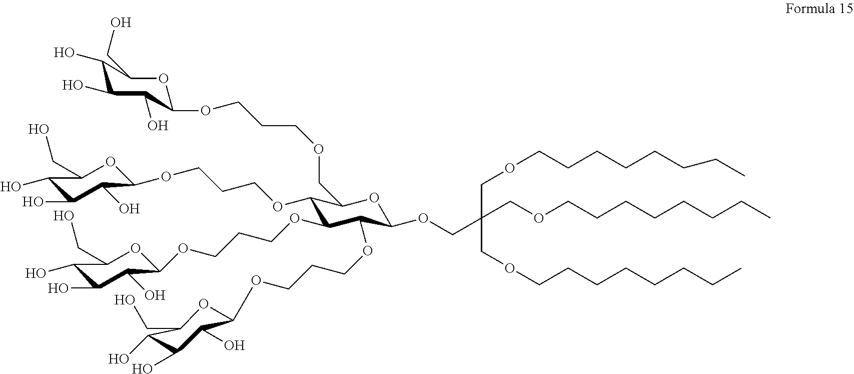

In another embodiment of the present invention, the expression “TPS-E9L” refers to a compound, wherein L represents a methylene group; Z represents —CH₂-A³-R³, wherein A¹ to A³ represent oxygen atoms and R¹ to R³ represent unsubstituted C₉ alkyl groups; and X represents a penta-saccharide, in which each of four glucose units is linked to a central glucose core via a propylene spacer. Therefore, the compound may be represented by Formula 16 below:

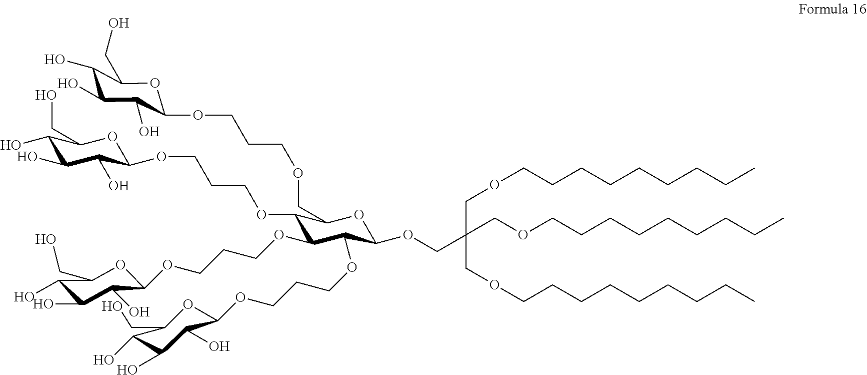

In another embodiment of the present invention, the expression “TPS-E10L” refers to a compound, wherein L represents a methylene group; Z represents —CH₂-A³-R³, wherein A¹ to A³ represent oxygen atoms and R¹ to R³ represent unsubstituted C₁₀ alkyl groups; and X represents a penta-saccharide, in which each of four glucose units is linked to a central glucose core via a propylene spacer. Therefore, the compound may be represented by Formula 17 below:

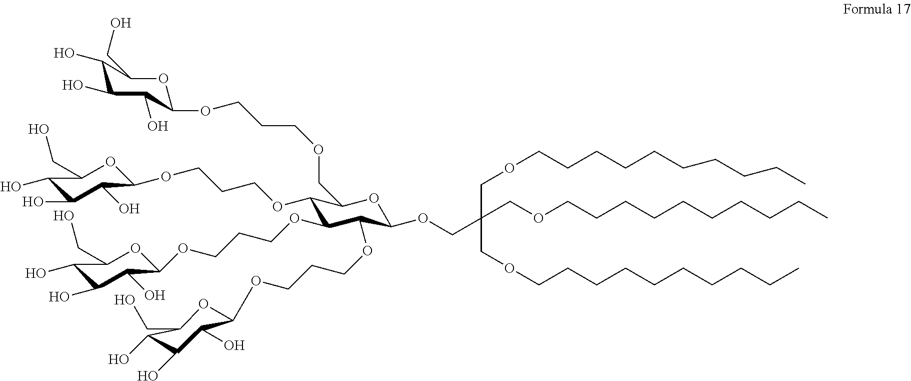

In another embodiment of the present invention, the expression “TPS-E11L” refers to a compound, wherein L represents a methylene group; Z represents —CH₂-A³-R³, wherein A¹ to A³ represent oxygen atoms and R¹ to R³ represent unsubstituted C₁₁ alkyl groups; and X represents a penta-saccharide, in which each of four glucose units is linked to a central glucose core via a propylene spacer. Therefore, the compound may be represented by Formula 18 below:

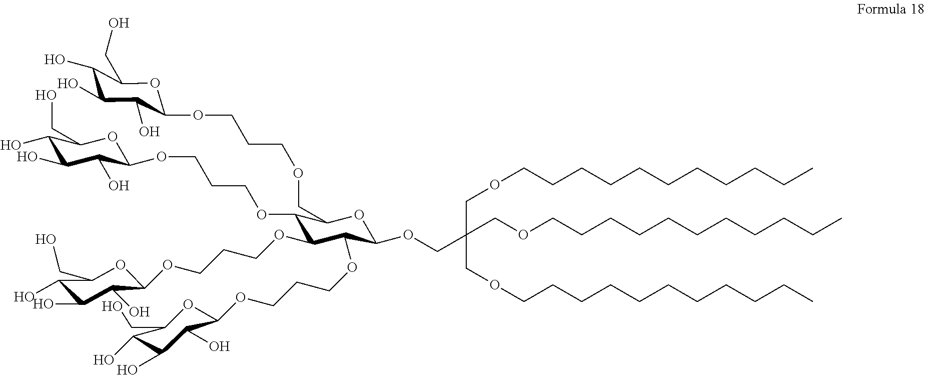

The compound according to another embodiment of the present invention may be an amphipathic molecule for extracting, solubilizing, stabilizing, crystallizing or analyzing membrane proteins, without being limited thereto.

As used herein, the term “amphipathic molecule” refers to a molecule that has both hydrophobic and hydrophilic groups and affinity for both polar and nonpolar solvents. Surfactants or phospholipid molecules present in the cell membrane are amphipathic substances having a hydrophilic group at one end and a hydrophobic group at the other end and are capable of forming micelles or liposomes in aqueous solutions. Since hydrophilic groups have polarity but nonpolar groups coexist, amphipathic molecules tend to be insoluble in water. However, when the concentration of amphipathic molecules is above a certain limiting concentration (i.e., critical micelle concentration, CMC), hydrophobic groups are gathered inward due to hydrophobic interactions and hydrophilic groups are exposed at the surface, generating micelles, which increases solubility in water.

Methods of measuring CMC are not particularly limited, and methods widely known in the art may be used. For example, a fluorescence staining method using diphenylhexatriene (DPH) may be used.

In an aqueous solution, the compound according to one embodiment of the present invention may have a critical micelle concentration (CMC) of 0.0001 to 1.0 mM, specifically, 0.0005 to 1.0 mM, more specifically, 0.0005 to 0.5 mM, still more specifically, 0.001 to 0.5 mM, and for example, the CMC may be 0.001 to 0.27 mM, without being limited thereto.

In the case of n-dodecyl-β-D-maltopyranoside (DDM), which has been conventionally used for membrane protein studies, the critical micelle concentration of DDM is 0.170 mM. Compared to this, PSAs, PSEs or TPSs according to embodiments of the present invention had a lower CMC value than DDM. Thus, since PSAs, PSEs or TPSs easily form micelles with small amounts, PSAs, PSEs or TPSs may be used to effectively study and analyze membrane proteins using small amounts as compared to DDM.

In accordance with an aspect of the present invention, the above and other objectives can be accomplished by the provision of a composition for extracting, solubilizing, stabilizing, crystallizing or analyzing membrane proteins including the compound.

The composition may be prepared in the form of micelles, liposomes, emulsions or nanoparticles, without being limited thereto.

The micelles may have a radius of 2.0 to 70.0 nm, specifically 2.0 to 45.0 nm; more specifically, the micelles formed by PSAs according to embodiments of the present invention may have a radius of 2.0 to 4.0 nm, for example, 2.5 to 3.5 nm; the micelles formed by PSEs according to another embodiment of the present invention may have a radius of 2.0 to 30.0 nm, for example, 2.6 to 15.0 nm: and the micelles formed by TPSs according to yet another embodiment of the present invention may have a radius of 2.0 to 70.0 nm, for example, 2.3 to 60.0 nm, without being limited thereto.

Methods of measuring the radius of micelles are not particularly limited, and methods widely known in the art may be used. For example, dynamic light scattering (DLS) may be used.

The micelles, liposomes, emulsions or nanoparticles may contain membrane proteins therein. That is, the micelles, liposomes, emulsions or nanoparticles may extract and enclose membrane proteins present in the cell membranes. Therefore, it is possible to extract, solubilize, stabilize, crystallize or analyze membrane proteins using the micelles.

The composition may further include buffers, which may aid extraction, solubilization, stabilization or analysis of membrane proteins.

In accordance with another aspect of the present invention, there is provided a method of preparing a compound represented by Formula 1 below, the method including:

1) a step of preparing dialkylated diethylmalonate by adding a 1-iodoalkane to diethyl malonate;

2) a step of preparing a dialkylated mono-ol by adding LiCl, DMSO and H₂O to the prepared dialkylated diethylmalonate, heating the mixture to a temperature of 150 to 200° C., and adding LiAlH₄ and THF to the mixture;

3) a step of introducing a protecting group-attached glucose by performing a glycosylation reaction on the prepared dialkylated mono-ol;

4) a step of removing an O-benzoyl group by performing a deprotection reaction on the product prepared in step 3);

5) a step of attaching four glucose units with attached protecting groups by performing a glycosylation reaction on the product prepared in step 4) to introduce a penta-saccharide hydrophilic group; and

6) a step of removing an O-benzoyl group by performing a deprotection reaction on the product prepared in step 5),

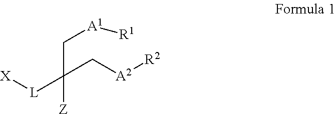

wherein L may represent a methylene group; A¹ and A² may represent methylene groups; each of R¹ and R² may independently represent a substituted or unsubstituted C₃-C₂₀ alkyl group, a substituted or unsubstituted C₃-C₂₀ cycloalkyl group, or a substituted or unsubstituted C₃-C₂₀ aryl group; Z may represent a hydrogen atom; and X may represent a glucose-centered branched penta-saccharide.

The method according to the above embodiment may be the method of preparing PSAs according to one embodiment of the present invention, without being limited thereto.

In this embodiment, the compound may be synthesized by a simple synthetic method consisting of six steps using diethyl malonate as a starting material. According to the method of the present invention, since synthesis of the compound is easy, mass production of the compound for membrane protein studies is possible.

In accordance with yet another aspect of the present invention, there is provided a method of preparing a compound represented by Formula 1 below, the method including:

1) a step of preparing an alcohol derivative by adding NaOH and an alcohol to epichlorohydrin;

2) a step of introducing a protecting group-attached glucose by performing a glycosylation reaction on the prepared alcohol derivative;

3) a step of removing an O-benzoyl group by performing a deprotection reaction on the product prepared in step 2);

4) a step of attaching four glucose units with attached protecting groups by performing a glycosylation reaction on the product prepared in step 3) to introduce a penta-saccharide hydrophilic group: and 5) a step of removing an O-benzoyl group by performing a deprotection reaction on the product prepared in step 4),

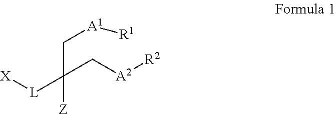

wherein L may represent a direct bond; A¹ and A² may represent oxygen atoms; each of R¹ and R² may independently represent a substituted or unsubstituted C₃-C₂₀ alkyl group, a substituted or unsubstituted C₃-C₂₀ cycloalkyl group, or a substituted or unsubstituted C₃-C₂₀ aryl group; Z may represent a hydrogen atom; and X may represent a glucose-centered branched penta-saccharide.

The method according to the above embodiment may be the method of preparing PSEs according to another embodiment of the present invention, without being limited thereto.

In this embodiment, the compound may be synthesized by a simple synthetic method consisting of five steps using epichlorohydrin as a starting material. According to the method of the present invention, since synthesis of the compound is easy, mass production of the compound for membrane protein studies is possible.

In accordance with yet another aspect of the present invention, there is provided a method of preparing a compound represented by Formula 1 below, the method including:

1) a step of preparing dialkylated diethylmalonate by adding a 1-iodoalkane to diethyl malonate;

2) a step of preparing a dialkylated diol by adding LiAlH₄ and THF to the prepared dialkylated diethylmalonate;

3) a step of adding an alkyl chain by adding a 1-bromoalkane to the prepared dialkylated diol;

4) a step of introducing a protecting group-attached glucose by performing a glycosylation reaction on the product prepared in step 3);

5) a step of removing an O-benzoyl group by performing a deprotection reaction on the product prepared in step 4);

6) a step of attaching four glucose units with attached protecting groups by performing a glycosylation reaction on the product prepared in step 5) to introduce a penta-saccharide hydrophilic group; and 7) a step of removing an O-benzoyl group by performing a deprotection reaction on the product prepared in step 6),

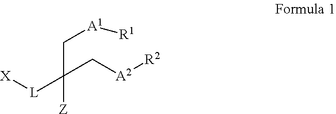

wherein L may represent a methylene group; Z may represent —CH₂-A³-R³, wherein one of A¹ to A³ may represent an oxygen atom and the others may represent methylene groups; each of R¹ to R³ may independently represent a substituted or unsubstituted C₃-C₂₀ alkyl group; and R¹ to R³ may be the same; and X may represent a glucose-centered branched penta-saccharide.

The method according to the above embodiment may be the method of preparing TPS-As according to another embodiment of the present invention, without being limited thereto.

In this embodiment, the compound may be synthesized by a simple synthetic method consisting of seven steps using diethyl malonate as a starting material. According to the method of the present invention, since synthesis of the compound is easy, mass production of the compound for membrane protein studies is possible.

In accordance with yet another aspect of the present invention, there is provided a method of preparing a compound represented by Formula 1 below, the method including:

1) a step of synthesizing a dialkylated diol using 5,5-bis-bromomethyl-2,2-dimethyl-[1,3]dioxane as a starting material;

2) a step of adding an alkyl chain by adding a 1-bromoalkane to the product prepared in step 1);

3) a step of introducing a protecting group-attached glucose by performing a glycosylation reaction on the product prepared in step 2);

4) a step of removing an O-benzoyl group by performing a deprotection reaction on the product prepared in step 3);

5) a step of attaching four glucose units with attached protecting groups by performing a glycosylation reaction on the product prepared in step 4) to introduce a penta-saccharide hydrophilic group; and 6) a step of removing an O-benzoyl group by performing a deprotection reaction on the product prepared in step 5),

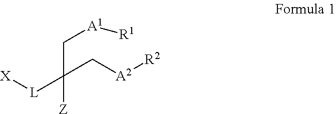

wherein L may represent a methylene group; Z may represent —CH₂-A³-R³, wherein one of A¹ to A³ may represent an oxygen atom and the others may represent methylene groups; each of R¹ to R³ may independently represent a substituted or unsubstituted C₃-C₂₀ alkyl group; and R¹ to R³ ma_{y} be the same; and X may represent a glucose-centered branched penta-saccharide.

The method according to the above embodiment may be the method of preparing TPS-Es according to another embodiment of the present invention, without being limited thereto.

In this embodiment, the compound may be synthesized by a simple synthetic method consisting of six steps using 5,5-bis-bromomethyl-2,2-dimethyl-[1,3]dioxane as a starting material. According to the method of the present invention, since synthesis of the compound is easy, mass production of the compound for membrane protein studies is possible.

In accordance with yet another aspect of the present invention, there is provided a method of extracting, solubilizing, stabilizing, crystallizing or analyzing membrane proteins, the method including a step of treating membrane proteins with a compound represented by Formula 1 below in an aqueous solution:

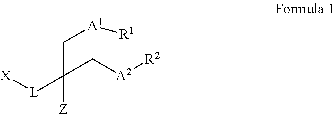

wherein L may represent a substituted or unsubstituted C₁-C₁₀ alkylene group or a direct bond;

Each of A¹ and A² may represent a methylene group or an oxygen atom;

Each of R¹ and R² may independently represent a substituted or unsubstituted C₃-C₂₀ alkyl group, a substituted or unsubstituted C₃-C₂₀ cycloalkyl group, or a substituted or unsubstituted C₃-C₂₀ aryl group;

X may represent a glucose-centered branched penta-saccharide linked by oxygen; and

Z may represent a hydrogen atom or —CH₂-A³-R³, wherein A³ may represent a methylene group or an oxygen atom, and R³ may independently represent a substituted or unsubstituted C₃-C₂₀ alkyl group, a substituted or unsubstituted C₃-C₂₀ cycloalkyl group, or a substituted or unsubstituted C₃-C₂₀ aryl group.

Specifically, L may represent a methylene group; A¹ and A² may represent methylene groups; each of R¹ and R² may independently represent a substituted or unsubstituted C₃-C₂₀ alkyl group, a substituted or unsubstituted C₃-C₂₀ cycloalkyl group, or a substituted or unsubstituted C₃-C₂₀ aryl group; and Z may represent a hydrogen atom. More specifically, L may represent a methylene group; A¹ and A² may represent methylene groups; R¹ and R² may represent substituted or unsubstituted C₅-C₁₅ alkyl groups; R¹ and R² may be the same; and Z may represent a hydrogen atom.

In particular, L may represent a direct bond; A¹ and A² may represent oxygen atoms; each of R¹ and R² may independently represent a substituted or unsubstituted C₃-C₂₀ alkyl group, a substituted or unsubstituted C₃-C₂₀ cycloalkyl group, or a substituted or unsubstituted C₃-C₂₀ aryl group; and Z may represent a hydrogen atom. More specifically, L may represent a direct bond; A¹ and A² may represent oxygen atoms; R¹ and R² may represent substituted or unsubstituted C₅-C₁₅ alkyl groups; R¹ and R² may be the same; and Z may represent a hydrogen atom.

In particular, L may represent a methylene group; and Z may represent —CH₂-A³-R³, wherein one or more of A¹ to A³ may represent oxygen atoms and the other(s) may represent methylene groups; each of R¹ to R³ may independently represent a substituted or unsubstituted C₃-C₂₀ alkyl group, a substituted or unsubstituted C₃-C₂₀ cycloalkyl group, or a substituted or unsubstituted C₃-C₂₀ aryl group; and R¹ to R³ may be the same. More specifically, L may represent a methylene group; and Z may represent —CH₂-A³-R³, wherein one or more of A¹ to A³ may represent oxygen atoms and the other(s) may represent methylene groups; each of R¹ to R³ may independently represent a substituted or unsubstituted C₃-C₂₀ alkyl group; and R¹ to R³ may be the same.

The compound may correspond to one of Formulas 2 to 18 according to one embodiment of the present invention, without being limited thereto.

As used herein, the term “membrane proteins” is a generic term for proteins or glycoproteins present in the lipid bilayer of the cell membrane. The membrane proteins exist in various states, such as passing through the cell membrane layer, located on the surface layer, or attached to the cell membrane. For example, the membrane proteins include enzymes, receptors for peptide hormones, local hormones, and the like, sugar transport channels, ion channels, and cell membrane antigens, without being limited thereto.

The membrane proteins include any proteins or glycoproteins present in the lipid bilayer of the cell membrane, and specifically may be boron transporter (BOR1), leucine transporter (LeuT), melibiose permease (MelB), human β2 adrenergic receptors (β2ARs), uric acid-xanthine/H+ symporter (UapA), or a combination of two or more thereof, without being limited thereto.

As used herein, the term “extraction of membrane proteins” refers to separating membrane proteins from the cell membranes.

As used herein, the term “solubilization of membrane proteins” refers to dissolving water-insoluble membrane proteins in micelles, amphipathic molecules, in an aqueous solution.

As used herein, the term “stabilization of membrane proteins” refers to stably preserving the tertiary or quaternary structure so that the structures and functions of the membrane proteins do not change.

As used herein, the term “crystallization of membrane proteins” refers to the formation of crystals of the membrane proteins in a solution.

As used herein, the term “analysis of membrane proteins” refers to analysis of the structures or functions of the membrane proteins. In the above embodiments, analysis of membrane proteins may be performed using known methods, without being limited thereto, and, for example, electron microscopy may be used to analyze the structures of membrane proteins.

The hydrophilic group of an amphipathic compound plays a very important role in membrane protein stabilization. For example, lauryldimethylamine-N-oxide (LDAO) and n-dodecyl-β-D-maltoside (DDM) have dodecyl chains in common, but contain N-oxide and maltoside head groups, respectively (see Newstead, S. et al., Protein Sci. 2008, 17, 466-472.). Despite the presence of the same tail group, these two amphipathic molecules have a very different ability to stabilize membrane proteins in a solution; LDAO has a somewhat lower ability to stabilize membrane proteins, whereas DDM has the highest ability to stabilize membrane proteins among 120 existing amphipathic molecules. Similar trends may be found in the comparison of glucoside (e.g., n-octyl-β-D-glucopyranoside (OG)) and maltoside (e.g., n-decyl-β-D-maltoside (DM) and DDM) amphipathic molecules. Maltoside amphipathic molecules are generally superior to glucoside amphipathic molecules in terms of membrane protein stabilization. Despite the importance of the hydrophilic group of an amphipathic molecule for achieving membrane protein stabilization, efforts to develop an amphipathic molecule with a new hydrophilic group have been limited to date. A new formulation, chobimalt, interestingly, contains a linear tetrasaccharide as a head group, but this formulation was only effective in stabilizing membrane proteins in the presence of existing amphipathic molecules. On the other hand, the novel carbohydrate-based hydrophilic group (i.e., branched penta-saccharide) introduced in the present invention has a multi-branched structure, which is distinct from chobimalt and existing amphipathic molecules. In this hydrophilic group, four glucose units are attached directly or via propylene spacers to a central glucose unit, and thus the hydrophilic group exhibits a specific three-dimensional structure, wherein five glucose units are densely interconnected. Generally, these high-density carbohydrates are very difficult to prepare, but the hydrophilic groups of penta-saccharides such as TPSs, PSAs and PSEs may be prepared in 4 to 6 steps at a total yield of 40 to 60%. Such a simple preparation method of the hydrophilic groups is advantageous for commercialization since the hydrophilic groups may be produced on a large scale. Among new agents, TPS-E8, TPS-E10L and PSE-C11, compared to the best existing amphipathic molecule DDM, provided significantly improved stability to all four membrane proteins tested (i.e., membrane proteins including eukaryotic membrane proteins such as BOR1 and β₂AR). These results confirmed the importance of the hydrophilic group of an amphipathic molecule in stabilizing membrane proteins. This novel branched penta-saccharide hydrophilic group may be used to design new amphipathic compounds.

This novel compound has branched alkyl chains with various lengths. Since the head group of the branched penta-saccharide has high hydrophilicity, a large hydrophobic group is required to maintain an optimal hydrophilic-lipophilic balance (HLB). When a linear alkyl chain is used as a hydrophobic group instead of a branched one, an amphipathic molecule with a very long alkyl chain will be generated. Theoretically, a linear alkyl chain with more than 20 carbons was required to balance a bulky penta-saccharide head group. However, an amphipathic molecule having a long linear alkyl chain may inhibit the stability of membrane proteins by mass-matching with the sizes of membrane proteins, and also produce large protein-detergent complexes (PDCs), which lowers efficiency. Thus, the amphipathic molecule having a long linear alkyl chain may not be suitable for membrane protein crystallization. Furthermore, starting materials (alcohol/halide derivatives) for preparing amphipathic molecules of this type are either commercially unavailable or very expensive. On the other hand, TPSs, PSAs and PSEs containing a branched alkyl chain form small PDCs with β₂AR, which is well suited for β₂AR crystallization. This is an advantage of this hydrophobic group. The branched alkyl group also plays an important role in membrane protein solubilization, and as demonstrated by TPAs studies, the number of hydrophobic groups is closely related to membrane protein solubilization. The novel compound according to the present invention was capable of extracting and solubilizing β₂AR in addition to a BOR1-GFP fusion protein and MelBₛₜ from the cell membranes.

TPS-E8, TPS-E10L and PSE-C11 according to one embodiment of the present invention were excellent in stabilizing and visualizing membrane protein complexes as exemplified by T4L-β₂AR-Gs or β₂AR-Gs complexes. Most membrane proteins are assembled with other proteins and exhibit their biological roles, and thus structural and functional studies on membrane protein complexes are very important, but very challenging. These difficulties are mainly related to conservation of the quaternary structure of these complexes. Very few amphipathic molecules are known to be suitable for long-term stabilization of eukaryotic protein complexes. MNG-3 is suitable for stabilizing complexes, but this formulation has a tendency to form large PDCs. On the other hand, TPS-E8, TPS-E10L and PSE-C11 tend to form small PDCs and are suitable for structural studies of membrane protein complexes. In addition, the newly developed amphipathic molecule was superior to MNG-3 in maintaining the original structure of membrane proteins. For example, at an amphipathic molecule concentration of CMC+0.2 wt %, LeuT solubilized in MNG-3 has its activity reduced to 40% during 12 days of incubation, whereas, in the case of TPS-E8 and PSE-C11, the transporter activity was completely preserved during the same period. Since one amphipathic molecule cannot be applied to various membrane proteins with different structures and properties, the development of a novel amphipathic molecule that has a structure different from existing amphipathic molecules and other new agents and that is capable of being applied to various membrane proteins is urgently needed for membrane protein research.

Preferred surfactant properties such as efficient protein solubilization, protein stabilization and formation of small PDCs often do not coexist within a single molecule. For example, highly efficient LDAO for solubilizing membrane proteins is less effective for membrane protein stabilization than DDM, but DDM is less effective than LDAO for membrane protein extraction. With respect to PDC size, DDM tends to form large PDCs, which often result in diffraction crystals with poor quality from target proteins prepared by this amphipathic molecule. On the other hand, LDAO tends to form small PDCs. When target proteins are sufficiently robust to be able to withstand structural degradation in the amphipathic molecule, LDAO is advantageous in terms of membrane protein crystallization. In the present invention, the inventors have identified PSE-C11 (and PSE-C13), which have a significant effect on membrane protein solubilization and stabilization compared to conventional amphiphilic molecules and form small PDCs with various membrane proteins. In addition, the present inventors demonstrated that PSE-C11 and TPS-E10L are suitable for structural studies of membrane proteins (and complexes thereof) through EM analysis. Therefore, these compounds have high potential as a tool for studying the structures and functions of membrane proteins. In addition, the molecular design principles employing the roles of the hydrophilic and hydrophobic groups of the amphiphilic molecule described in the present invention will facilitate the development of new amphipathic compounds in the future.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 illustrates the synthetic scheme of PSAs according to Example 1 of the present invention;

FIG. 2 illustrates the chemical structure of PSAs according to examples of the present invention;

FIG. 3 illustrates the synthetic scheme of PSEs according to Example 2 of the present invention;

FIG. 4 illustrates the chemical structure of PSEs according to examples of the present invention;

FIG. 5 illustrates the synthetic schemes and chemical structures of TPS-Es and TPS-ELs according to Examples 3 and 4 of the present invention;

FIG. 6 illustrates the synthetic scheme and chemical structure of TPS-As according to Example 5 of the present invention;

FIG. 7 includes graphs showing the size (diameter (D), nm) distribution of micelles formed by PSAs and PSEs;

FIG. 8 includes graphs showing the size (diameter (D), nm) distribution of micelles formed by TPSs (TPS-As, TPS-Es and TPS-ELs);

FIG. 9 shows the results of measuring structural stability of a BOR1-GFP protein solubilized in (a) PSAs (PSA-C9, PSA-C10, PSA-C11) or (b) PSEs (PSE-C9, PSA-C11, PSE-C13) compared to DDM;

FIG. 10 shows the results of measuring structural stability of a BOR1-GFP protein after heating the BOR1-GFP protein solubilized in (a) DDM or (b) PSE-C11 to respective temperatures (35, 40, 45 or 50° C.);

FIG. 11 shows the results of measuring the stability of a LeuT protein by PSAs or PSEs with a (a) CMC+0.04 wt % or (b) CMC+0.2 wt % concentration using scintillation proximity assay (SPA);

FIG. 12 shows the results of measuring the stability of a LeuT protein by TPS-As or TPS-Es with a (a) CMC+0.04 wt % or (b) CMC+0.2 wt % concentration using scintillation proximity assay (SPA);

FIG. 13 shows the results of measuring the stability of a LeuT protein by TPS-ELs with a (a) CMC+0.04 wt % or (b) CMC+0.2 wt % concentration using a scintillation proximity assay (SPA);

FIG. 14 shows the results of measuring the extraction efficiency and structural stability of a MelB protein by 1.5 wt % PSAs or PSEs at respective temperatures (0, 45, 55 or 65° C.) using SDS-PAGE and western immunoblotting;

FIG. 15 shows the results of measuring structural stability of mBBr-β₂AR solubilized in PSAs or PSEs in the presence of a high-affinity agonist BI (BI-167107) using a bimane fluorescence spectrum;

FIG. 16 shows the results of measuring the structural change and structural stability of mBBr-β₂AR solubilized in PSAs/PSEs or DDM depending on the presence or absence of a full agonist (isoproterenol, ISO) or the combination of ISO and a G-protein;

FIG. 17 (a) shows the results of measuring the activity of a receptor (mBBr-β₂AR) solubilized in DDM, PSAs or PSEs and the receptor activity was measured by binding of [³H]-dihydroalprenolol ([³H]-DHA). FIG. 17 (b) shows the results of measuring the sizes of β₂AR complexes formed by these amphipathic molecules using size exclusion chromatography (SEC);

FIG. 18 shows the results of measuring the initial activity of a receptor (β₂AR) solubilized in DDM, TPS-As, TPS-Es or TPS-ELs and the initial activity was measured by binding of [³H]-dihydroalprenolol ([³H]-DHA);

FIG. 19 shows the results of confirming whether a receptor (β₂AR) solubilized in DDM, TPS-A8 or TPS-E8 retained the activity thereof for a long period of time and the activity was determined by binding of [³H]-dihydroalprenolol ([³H]-DHA);

FIG. 20 (a) shows the results of confirming whether a receptor (β₂AR) solubilized in DDM or TPS-ELs retained the activity thereof for a long period of time and the activity was determined by binding of [³H]-dihydroalprenolol ([³H]-DHA). FIG. 20 (b) shows the results of measuring the sizes of β₂AR complexes formed by these amphipathic molecules using size exclusion chromatography (SEC);

FIG. 21 shows negative staining electron microscopy (EM) images of β₂AR purified by (a) DDM, (b) PSA-C11, (c) PSE-C11, or (d) PSE-C13;

FIG. 22 shows the activity of β₂AR receptors extracted and solubilized directly from the cell membranes using 1.0 wt % PSE-C11 or DDM, and the receptor activity was measured using a radiolabeled ligand, [³H]-DHA;

FIG. 23 (a) and (b) show the results of measuring the size of β₂AR via size exclusion chromatography (SEC) using a buffer solution containing an amphipathic compound or a buffer solution not containing an amphipathic compound. β₂AR receptors were extracted directly from the cell membranes using 1.0 wt % (a) DDM or (b) PSE-C11. FIG. 23 (c) shows the results of confirming whether T4L-β₂AR-Gₛ complexes solubilized in PSE-C11 retained structural stability thereof for a long period of time. The structural stability was measured in a buffer solution containing PSE-C11 using size exclusion chromatography (SEC). On day 15, the results were measured using both the buffer solution containing PSE-C11 and the buffer solution not containing PSE-C11;

FIG. 24 shows (a) the raw EM images of the single particles of T4L-β₂AR-Gₛ complexes purified using PSE-C11, (b) the 2D classification images and (c) the representative class average images of complexes with the same orientation;

FIG. 25 shows (a) the raw EM images of the single particles of β₂AR-Gₛ complexes purified using TPS-E10L, (b) the 2D classification images and (c) the representative class average images of complexes with the same orientation;

FIG. 26 shows the results of measuring the thermal stability of UapA in an aqueous solution when TPS-As/Es, MNG-3 or DDM was used. The thermal stability was determined using CPM analysis:

(a) represents a case where TPS-As/Es, MNG-3 or DDM with a concentration of CMC+0.04 wt % was used, and

(b) represents a case where TPS-As/Es, MNG-3 or DDM with a concentration of CMC+0.2 wt % was used; and

FIG. 27 shows the results of measuring the thermal stability of UapA in an aqueous solution when TPS-ELs, MNG-3 or DDM was used. The thermal stability was determined using CPM analysis:

(a) represents a case where TPS-ELs, MNG-3 or DDM with a concentration of CMC+0.04 wt % was used, and

(b) represents a case where TPS-ELs, MNG-3 or DDM with a concentration of CMC+0.2 wt % was used.

### DETAILED DESCRIPTION OF EXEMPLARY EMBODIMENTS

Hereinafter, the present invention is described in more detail with reference to the following examples. It should be understood, however, that the following examples are illustrative only and do not limit the scope of the invention. Modifications and variations that those skilled in the art can easily deduce from the description and embodiments of the present invention will be construed as within the scope of the present invention.

### EXAMPLE 1

### Method of Synthesizing Alkyl-Based Penta-Saccharide Amphiphiles (PSAs)

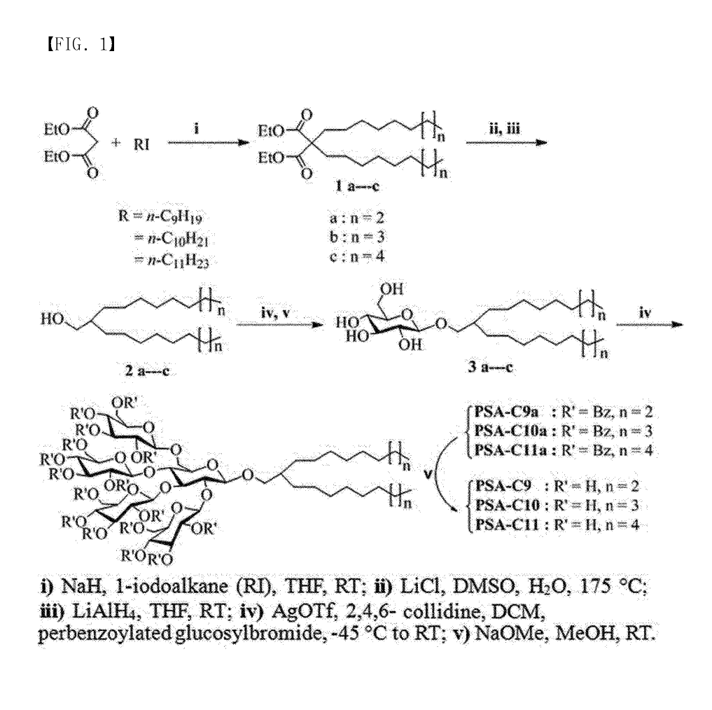

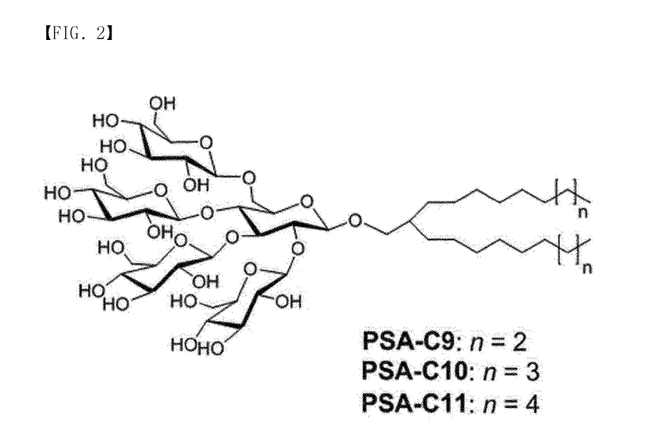

A synthetic scheme for PSAs is shown in FIG. 1. Three kinds of alkyl-based penta-saccharide amphiphiles (PSAs) were synthesized according to methods described in the following <1-1> to <1-5>, and the synthesized PSAs are shown in FIG. 2.

<1-1> General Synthetic Procedures for dialkylated diethylmalonate (Synthesis of Compounds 1a to 1c)

The method used to carry out this reaction is a modification of the method described in the journal article (P. S. Chae et al., Nat. Methods 2010, 7, 1003-1008.) published by the present inventors.

Specifically, NaH (30 mmol) dissolved in tetrahydrofuran (THF) was added to a diethyl malonate (10 mmol) solution dissolved in THF (40 mL) at 0° C. and the mixture was stirred for 20 minutes. After adding a 1-iodoalkane (25 mmol), the reaction mixture was stirred at room temperature for 48 hours, then the reaction was terminated by adding a cold saturated aqueous NH₄Cl solution, and extraction was performed using diethyl ether. An organic layer was washed with brine and dried using anhydrous Na₂SO₄. After complete evaporation of the solvent, residues were purified using silica-gel column chromatography (EtOAc/hexane) to obtain dialkylated diethylmalonate (compounds 1a to 1c) as an oily liquid.

<1-2> General Synthetic Procedures of dialkylated mono-ol (Synthesis of Compounds 2a to 2c)

LiCl (15.2 mmol) and H₂O (7.7 mmol) were added to a dialkylated malonate (1a-c; 6.9 mmol) solution dissolved in DMSO. The mixture was heated to 175° C. for 12 hours, then cooled to room temperature and diluted with H₂O. The mixture was extracted with diethyl ether to obtain an organic layer. The obtained organic layer was washed with water and brine, and dried with anhydrous Na₂SO₄. After complete evaporation of the solvent, residues were dissolved in THF (30 mL) and LiAlH₄ (21.3 mmol) was slowly added thereto at 0° C. The mixture was stirred at room temperature for 4 hours, the reaction was terminated by the continuous addition of MeOH, water, and a 1N aqueous HCl solution at 0° C., and extraction was performed twice using diethyl ether. An obtained organic layer was washed with brine and dried with anhydrous Na₂SO₄. The reaction mixture was purified using silica-gel column chromatography (EtOAc/hexane) to obtain dialkyl-containing mono-ols (compounds 2a to 2c) as an oily liquid (yield of 80 to 86% (two steps)).

<1-3> General Procedures for glycosylation Reaction, and de-O-benzoylation Reaction Under Zemplen Conditions (Synthesis of Compounds 3a to 3c)

The method used to carry out this reaction is a modification of the method described in the journal article (P.S. Chae et al., Chem. Eur. J. 2013, 19, 15645-15651) published by the present inventors.

Specifically, a glycosylation reaction was performed as follows. A mixture of mono-ol derivatives (compounds 2a to 2c) dissolved in anhydrous CH₂Cl₂ (30 mL), AgOTf (1.2 equiv.) and 2,4,6-collidine (0.7 equiv.) was stirred at −45° C. Next, perbenzoylated glucosylbromide (1.2 equiv.) dissolved in CH₂Cl₂ (30 mL) was slowly added to the suspension over 10 minutes and then the reaction mixture was allowed to slowly come to 0° C. Progress of the reaction was monitored by TLC. After completion of the reaction (as determined by TLC), pyridine was added to the reaction mixture. The reaction mixture was diluted with CH₂Cl₂ (30 mL) and filtered through Celite. The filtrate was washed successively with a 1M Na₂S₂O₃ aqueous solution (30 mL), 0.1M HCl aqueous solution (30 mL) and brine (30 mL). Then, an organic layer was dried with anhydrous Na₂SO₄, and the solvent was removed using a rotary evaporator.

A de-O-benzoylation (de-O-benzoylation) reaction was performed as follows. Glycosylated residues were dissolved in MeOH and then a methanolic solution of 0.5 M NaOMe was added in a required amount so that the final concentration of NaOMe was 0.05 M. The reaction mixture was stirred at room temperature for 6 hours and then neutralized with Amberlite IR-120 (H⁺ form) resin. The resin was removed by filtration, washed with MeOH, and then the solvent was removed from the filtrate in vacuo. The residues were purified using silica-gel column chromatography (MeOH/CH₂Cl₂) to obtain products (compounds 3a to 3c) in the form of a white solid (yield of 84 to 88% (two steps)).

<1-4> Glycosylation Reaction (Synthesis of PSA-C9a to PSA-C11a)

PSA-C9a to PSA-C11a were prepared from compounds 3a to 3c in the same manner as the glycosylation reaction of Example 1-3.

Specifically, a mixture of compounds (compounds 3a to 3c) dissolved in anhydrous CH₂Cl₂ (30 mL), AgOTf (4.5 equiv.) and 2,4,6-collidine (2.0 equiv.) was stirred at −45° C. Next, perbenzoylated glucosylbromide (4.5 equiv.) dissolved in CH₂Cl₂ (30 mL) was slowly added to the suspension over 30 minutes and then the reaction mixture was allowed to slowly come to 0° C. The reaction was monitored by TLC. After completion of the reaction (as determined by TLC), pyridine was added to the reaction mixture. The reaction mixture was diluted with CH₂Cl₂ (30 mL) and filtered through Celite. The filtrate was washed successively with a 1M Na₂S₂O₃ aqueous solution (30 mL), 0.1M HCl aqueous solution (30 mL) and brine (30 mL). Then, an organic layer was dried with anhydrous Na₂SO₄, and the solvent was removed using a rotary evaporator.

<1-5> De-O-benzoylation Reaction Under Zemplen Conditions (Synthesis of PSA-C9 to PSA-C11)

PSA-C9 to PSA-C11 were prepared from PSA-C9a to PSA-C11a in the same manner as the de-O-benzoylation reaction of Example 1-3.

Specifically, the glycosylated residues of Example 1-4 were dissolved in MeOH and then a methanolic solution of 0.5 M NaOMe was added in a required amount so that the final concentration of NaOMe was 0.05 M. The reaction mixture was stirred at room temperature for 6 hours and then neutralized with Amberlite IR-120 (H⁺ form) resin. The resin was removed by filtration, washed with MeOH, and then the solvent was removed from the filtrate in vacuo. The residues were purified using silica-gel column chromatography (MeOH/CH₂Cl₂) to obtain PSA-C9 to PSA-C11.

### PREPARATION EXAMPLE 1

### Synthesis of PSA-C9

<1-1> Synthesis of diethyl 2,2-dinonylmalonate (Compound 1a)

According to the general synthetic procedures for dialkylated diethylmalonate described in Example 1-1, diethyl 2,2-dinonylmalonate (compound 1a) was prepared in a yield of 90% using 1-iodononane as a 1-iodoalkane. ¹H NMR (400 MHz, CDCl₃): δ 4.16 (q, J=8.0 Hz, 4H), 1.85 (q, J=8.8 Hz, 4H), 1.30-1.21 (m, 28H), 1.16 (t, J=8.0 Hz, 6H), 0.87 (t, J=8.0 Hz, 6H); ¹³C NMR (100 MHz, CDCl₃): δ 172.3, 61.1, 57.7, 32.2, 32.1, 31.8, 30.0, 29.8, 29.7, 29.5, 24.0, 22.9, 14.3.

<1-2> Synthesis of 2-nonylundecan-1-ol (Compound 2a)

According to the general synthetic procedures for a dialkylated mono-ol described in Example 1-2, 2-nonylundecan-1-ol (compound 2a) was prepared in a yield of 82%. ¹H NMR (400 MHz, CDCl₃): δ 3.54 (d, J=4.0 Hz, 2H), 1.50-1.40 (m, 1H), 1.37-1.20 (m, 32H), 0.88 (t, J=8.0 Hz, 6H); ¹³C NMR (100 MHz, CDCl₃): δ 65.7, 40.5, 31.9, 30.9, 30.1, 29.6, 29.3, 26.9, 22.7, 14.1.

<1-3> Synthesis of dimethyl 2-nonylmalonate (Compound 3a)

According to the general procedures for glycosylation and de-O-benzoylation described in Example 1-3, compound 3a was prepared in a yield of 86%. ¹H NMR (400 MHz, CD₃OD): δ 4.22 (d, J=8.0 Hz, 1H), 3.85-3.82 (m, 2H), 3.72-3.66 (m, 1H), 3.40-3.30 (m, 3H), 3.26-3.15 (m, 2H), 1.61 (br s, 1H), 1.38 (s, 2H), 1.30-1.26 (m, 30H), 0.90 (t, J=8.0 Hz, 6H); ¹³C NMR (100 MHz, CD₃OD): δ 104.8, 78.1, 77.8, 75.1, 74.1, 71.6, 62.8, 39.6, 33.2, 32.3, 31.3, 30.9, 30.6, 27.9, 23.9, 14.7.

<1-4> Synthesis of PSA-C9a

According to the glycosylation method described in Example 1-4, PSA-C9a was synthesized. Yield: 75%; ¹H NMR (400 MHz, CDCl₃): δ 8.26 (d, J=8.0 Hz, 2H), 8.20-7.61 (m, 30H), 7.60-7.55 (m, 2H), 7.43-7.18 (m, 46H), 5.95 (t, J=8.0 Hz, 1H), 5.90-5.81 (m, 3H), 5.80-5.70 (m, 2H), 5.60-5.45 (m, 6H), 4.99-4.80 (m, 5H), 4.72-4.62 (d, J=8.0 Hz, 2H), 4.60-4.50 (m, 4H), 4.40-4.32 (m, 1H), 4.20-4.00 (m, 5H), 3.92 (t, J=8.0 Hz, 1H), 3.82-3.75 (m, 3H), 3.68-3.64 (m, 1H), 3.39-3.32 (m, 1H), 3.12-3.01 (m, 1H), 2.91-2.88 (m, 1H), 2.71-2.65 (m, 1H), 1.32-1.10 (m, 32H), 0.84 (t, J=8.0 Hz, 6H); ¹³C NMR (100 MHz, CDCl₃): δ 166.2, 166.1, 166.0, 165.9, 165.8, 165.7, 165.2, 165.1, 164.9, 164.5, 164.4, 133.6, 133.4, 133.3, 133.2, 133.0, 130.2, 129.9, 129.8, 129.7, 129.6, 129.5, 129.3, 129.1, 129.0, 128.9, 128.6, 128.5, 128.4, 128.3, 128.2, 101.4, 100.5, 99.9, 99.8, 78.0, 75.9, 74.9, 73.2, 73.0, 72.8, 72.5, 72.4, 72.0, 71.8, 71.2, 70.6, 70.3, 70.0, 69.2, 38.3 32.0, 31.2, 30.9, 30.5, 30.4, 30.0, 29.9, 29.8, 29.5, 27.0, 26.8, 22.7, 14.2.

<1-5> Synthesis of PSA-C9

According to the de-O-benzoylation method described in Example 1-5, PSA-C9 was synthesized. Yield: 91%; ¹H NMR (400 MHz, CD₃OD): δ 4.97 (d, J=8.0 Hz, 1H), 4.79 (d, J=8.0 Hz, 1H), 4.68 (d, J=8.0 Hz, 1H), 4.46 (d, J=8.0 Hz, 1H), 4.40 (d, J=8.0 Hz, 1H), 4.28 (d, J=8.0 Hz, 1H), 4.09 (t, J=8.0 Hz, 1H), 3.90-3.78 (m, 8H), 3.70-3.62 (m, 5H), 3.45-3.27 (m, 18H), 1.60 (br s, 1H), 1.39-1.20 (m, 32H), 0.90 (t, J=8.0 Hz, 6H); ¹³C NMR (100 MHz, CD₃OD): δ 104.7, 103.6, 103.3, 102.3, 81.7, 79.8, 78.3, 78.1, 77.8, 76.1, 75.9, 75.2, 75.1, 75.0, 74.5, 71.6, 63.1, 62.9, 62.5, 39.5, 33.2, 31.3, 30.9, 30.6, 28.0, 27.8, 23.8, 14.6; HRMS (EI): calcd. for C₅₀H₉₂O₂₆[M+Na]³¹ 1131.5775, found 1131.5778.

### PREPARATION EXAMPLE 2

### Synthesis of PSA-C10

<2-1> Synthesis of diethyl 2,2-didecylmalonate (Compound 1b)

According to the general synthetic procedures for dialkylated diethylmalonate described in Example 1-1, diethyl 2,2-didecylmalonate (compound 1b) was prepared in a yield of 92% using 1-iododecane as a 1-iodoalkane. ¹H NMR (400 MHz, CDCl₃): δ 4.16 (q, J=8.0 Hz, 4H), 1.85 (q, J=8.8 Hz, 4H), 1.30-1.21 (m, 32H), 1.16 (t, J=8.0 Hz, 6H), 0.87 (t, J=8.0 Hz, 6H); ¹³C NMR (100 MHz, CDCl₃): δ 172.3, 61.1, 57.7, 32.2, 32.1, 30.0, 29.8, 29.7, 29.5, 24.0, 22.9, 14.3.

<2-2> Synthesis of 2-decyldodecan-1-ol (Compound 2b)

According to the general synthetic procedures for a dialkylated mono-ol described in Example 1-2, 2-decyldodecan-1-ol (compound 2b) was prepared in a yield of 86%. ¹H NMR (400 MHz, CDCl₃): δ 3.55 (d, J=4.0 Hz, 2H), 1.50-1.40 (m, 1H), 1.37-1.20 (m, 36H), 0.88 (t, J=8.0 Hz, 6H); ¹³C NMR (100 MHz, CDCl₃): δ 65.6, 40.5, 31.9, 30.9, 30.1, 29.7, 29.3, 26.9, 22.7, 14.0.

<2-3> Synthesis of Compound 3b

According to the general procedures for glycosylation and de-O-benzoylation described in Example 1-3, compound 3b was prepared in a yield of 88%. ¹H NMR (400 MHz, CD₃OD): δ 4.22 (d, J=8.0 Hz, 1H), 3.85-3.82 (m, 2H), 3.72-3.66 (m, 1H), 3.39-3.30 (m, 3H), 3.26-3.15 (m, 2H), 1.61 (br s, 1H), 1.38 (s, 2H), 1.30-1.26 (m, 34H), 0.90 (t, J=8.0 Hz, 6H); ¹³C NMR (100 MHz, CD₃OD): δ 104.8, 78.1, 77.8, 75.1, 74.1, 71.6, 62.82, 39.6, 33.2, 32.3, 32.2, 31.3, 30.9, 30.9, 30.6, 27.9, 23.9, 14.7.

<2-4> Synthesis of PSA-C10a

According to the glycosylation method described in Example 1-4, PSA-C10a was synthesized. Yield: 75%; ¹H NMR (400 MHz, CDCl₃): δ 8.26 (d, J=8.0 Hz, 2H), 8.20-7.61 (m, 30H), 7.60-7.55 (m, 2H), 7.43-7.18 (m, 46H), 5.95 (t, J=8.0 Hz, 1H), 5.90-5.81 (m, 3H), 5.80-5.70 (m, 2H), 5.60-5.45 (m, 6H), 4.99-4.80 (m, 5H), 4.72-4.62 (d, J=8.0 Hz, 2H), 4.60-4.50 (m, 4H), 4.40-4.32 (m, 1H), 4.20-4.00 (m, 5H), 3.92 (t, J=8.0 Hz, 1H), 3.82-3.75 (m, 3H), 3.68-3.64 (m, 1H), 3.39-3.32 (m, 1H), 3.12-3.01 (m, 1H), 2.91-2.88 (m, 1H), 2.71-2.65 (m, 1H), 1.32-1.09 (m, 36H), 0.84 (t, J=8.0 Hz, 6H); ¹³C NMR (100 MHz, CDCl₃): δ 166.2, 166.1, 166.0, 165.9, 165.8, 165.7, 165.2, 165.2, 165.1, 164.9, 164.5, 164.4, 133.6, 133.4, 133.3, 133.2, 133.0, 130.2, 129.9, 129.8, 129.7, 129.6, 129.5, 129.3, 129.1, 129.0, 128.9, 128.6, 128.5, 128.4, 128.3, 128.2, 101.4, 100.5, 99.9, 99.8, 78.0, 75.9, 74.9, 73.2, 73.0, 72.8, 72.5, 72.4, 72.0, 71.8, 71.2, 70.6, 70.3, 70.0, 69.2, 38.3, 32.0, 31.2, 30.9, 30.5, 30.3, 30.0, 29.9, 29.8, 29.5, 27.0, 26.8, 22.8, 14.2.

<2-5> Synthesis of PSA-C10

According to the de-O-benzoylation method described in Example 1-5, PSA-C10 was synthesized. Yield: 92%; ¹H NMR (400 MHz, CD₃OD): δ 4.97 (d, J=8.0 Hz, 1H), 4.79 (d, J=8.0 Hz, 1H), 4.68 (d, J=8.0 Hz, 1H), 4.46 (d, J=8.0 Hz, 1H), 4.40 (d, J=8.0 Hz, 1H), 4.28 (d, J=8.0 Hz, 1H), 4.09 (t, J=8.0 Hz, 1H), 3.90-3.78 (m, 8H), 3.70-3.61 (m, 5H), 3.45-3.26 (m, 18H), 1.60 (br s, 1H), 1.42-1.20 (m, 36H), 0.90 (t, J=8.0 Hz, 6H); ¹³C NMR (100 MHz, CD₃OD): δ 104.7, 103.6, 103.3, 102.3, 81.7, 79.8, 78.3, 78.1, 77.8, 76.1, 75.9, 75.2, 75.1, 75.0, 74.5, 71.6, 63.1, 62.9, 62.5, 39.5, 33.2, 31.3, 30.9, 30.6, 28.0, 27.8, 23.8, 14.6; HRMS (EI): calcd. for C₅₂H₉₆O₂₆ [M+Na]⁻ 1159.6088, found 1159.6086.

### PREPARATION EXAMPLE 3

### Synthesis of PSA-C11

<3-1> Synthesis of diethyl 2,2-diundecylmalonate (Compound 1c)

According to the general synthetic procedures for dialkylated diethylmalonate described in Example 1-1, diethyl 2,2-diundecylmalonate (compound 1c) was prepared in a yield of 90% using 1-iodoundodecane as a 1-iodoalkane. ¹H NMR (400 MHz, CDCl₃): δ 4.16 (q, J=8.0 Hz, 4H), 1.85 (q, J=8.8 Hz, 4H), 1.30-1.21 (m, 36H), 1.16 (t, J=8.0 Hz, 6H), 0.87 (t, J=8.0 Hz, 6H); ¹³C NMR (100 MHz, CDCl3): δ 172.2, 61.1, 57.7, 32.2, 32.1, 30.0, 29.8, 29.8, 29.5, 29.5, 24.1, 22.9, 14.3.

<3-2> Synthesis of 2-undecyltridecan-1-ol (Compound 2c)

According to the general synthetic procedures for a dialkylated mono-ol described in Example 1-2, 2-undecyltridecan-1-ol (compound 2c) was prepared in a yield of 85%. ¹H NMR (400 MHz, CDCl₃): δ 3.55 (d, J=4.0 Hz, 2H), 1.50-1.40 (m, 1H), 1.37-1.20 (m, 40H), 0.88 (t, J=8.0 Hz, 6H); ¹³C NMR (100 MHz, CDCl₃): δ 65.7, 40.6, 32.1, 31.1, 30.3, 29.9, 29.8, 29.5, 27.0, 22.8, 14.2.

<3-3> Synthesis of Compound 3c

According to the general procedures for glycosylation and de-O-benzoylation described in Example 1-3, compound 3c was prepared in a yield of 88%. ¹H NMR (400 MHz, CD₃OD): δ 4.22 (d, J=8.0 Hz, 1H), 3.85-3.82 (m, 2H), 3.72-3.66 (m, 1H), 3.39-3.30 (m, 3H), 3.26-3.15 (m, 2H), 1.61 (br s, 1H), 1.38 (s, 2H), 1.30-1.26 (m, 38H), 0.89 (t, J=8.0 Hz, 6H); ¹³C NMR (100 MHz, CD₃OD): δ 104.8, 78.2, 77.8, 75.1, 74.1, 71.6, 62.8, 39.6, 33.2, 32.3, 32.2, 31.3, 31.0, 30.9, 30.9, 30.6, 27.9, 23.9, 14.7.

<3-4> Synthesis of PSA-C11a

According to the glycosylation method described in Example 1-4, PSA-C11a was synthesized. Yield: 72%; ¹H NMR (400 MHz, CDCl₃): δ 8.26 (d, J=8.0 Hz, 2H), 8.20-7.61 (m, 30H), 7.60-7.55 (m, 2H), 7.45-7.20 (m, 46H), 5.95 (t, J=8.0 Hz, 1H), 5.90-5.81 (m, 3H), 5.80-5.70 (m, 2H), 5.60-5.45 (m, 6H), 4.99-4.80 (m, 5H), 4.72-4.62 (d, J=8.0 Hz, 2H), 4.60-4.50 (m, 4H), 4.40-4.32 (m, 1H), 4.20-4.00 (m, 5H), 3.92 (t, J=8.0 Hz, 1H), 3.82-3.75 (m, 3H), 3.68-3.64 (m, 1H), 3.39-3.32 (m, 1H), 3.12-3.01 (m, 1H), 2.91-2.88 (m, 1H), 2.71-2.65 (m, 1H), 1.35-1.09 (m, 40H), 0.84 (t, J=8.0 Hz, 6H); ¹³C NMR (100 MHz, CDCl₃): δ 166.2, 166.1, 166.0, 165.9, 165.8, 165.7, 165.2, 165.2, 165.1, 164.9, 164.5, 164.4, 133.6, 133.4, 133.3, 133.2, 133.0, 130.2, 129.9, 129.8, 129.7, 129.6, 129.5, 129.3, 129.1, 129.0, 128.9, 128.6, 128.5, 128.4, 128.3, 128.2, 101.4, 100.5, 99.9, 99.8, 78.0, 75.9, 74.9, 73.2, 73.0, 72.8, 72.5, 72.4, 72.0, 71.8, 71.2, 70.6, 70.3, 70.0, 69.2, 38.3, 32.0, 31.2, 30.9, 30.5, 30.4, 30.0, 29.9, 29.8, 29.4, 27.0, 26.7, 22.8, 14.2.

<3-5> Synthesis of PSA-C11

According to the de-O-benzoylation method described in Example 1-5, PSA-C11 was synthesized. Yield: 90%; ¹H NMR (400 MHz, CD₃OD): δ 4.97 (d, J=8.0 Hz, 1H), 4.79 (d, J=8.0 Hz, 1H), 4.68 (d, J=8.0 Hz, 1H), 4.46 (d, J=8.0 Hz, 1H), 4.40 (d, J=8.0 Hz, 1H), 4.28 (d, J=8.0 Hz, 1H), 4.09 (t, J=8.0 Hz, 1H), 3.90-3.78 (m, 8H), 3.70-3.62 (m, 5H), 3.45-3.26 (m, 18H), 1.60 (br s, 1H), 1.42-1.20 (m, 40H), 0.90 (t, J=8.0 Hz, 6H); ¹³C NMR (100 MHz, CD₃OD): δ 104.8, 103.6, 103.4, 102.4, 81.7, 79.8, 78.3, 78.1, 77.8, 76.1, 75.9, 75.2, 75.1, 75.0, 74.5, 71.7,71.3, 69.6, 63.1, 62.9, 62.6, 39.5, 33.2, 31.3, 30.9, 30.6, 28.0, 27.8, 23.9, 14.6; HRMS (EI): calcd. for C₅₄H₁₀₀O₂₆[M+Na]⁺ 1187.6401, found 1187.6396.

### EXAMPLE 2

### Synthesis of ether-based penta-saccharide amphiphiles (PSEs)

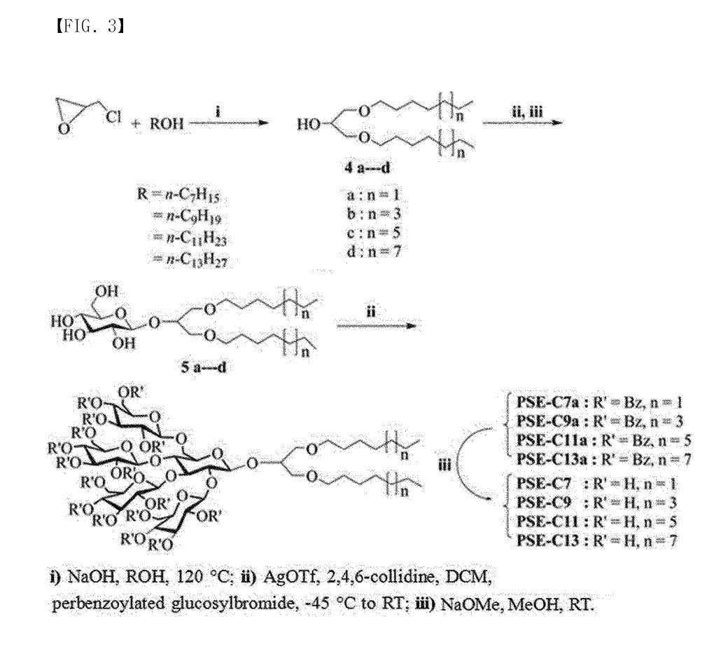

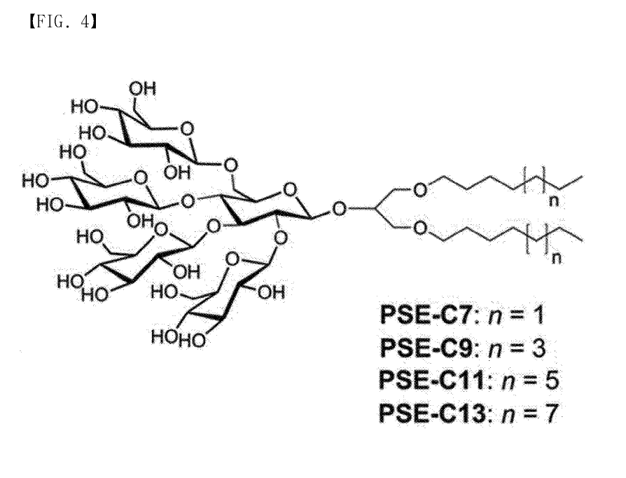

A synthetic scheme for PSEs is shown in FIG. 3. Three kinds of ether-based penta-saccharide amphiphiles (PSEs) were synthesized according to methods described in the following <2-1> to <2-4>, and the synthesized PSEs are shown in FIG. 4.

<2-1> General Synthetic Procedures for Alcohol Derivatives (Synthesis of Compounds 4a to 4d)

This reaction was performed by modifying the method described in the published journal article (Atilla, D. et al., J. Coord. Chem. 2009. 62, 3050-3059).

Specifically, epichlorohydrin (0.124 mmol) was added to an alcohol solution (0.43 mmol) with NaOH (0.25 mmol) under argon. The mixture was heated to 120° C. and stirred at the same temperature for 16 hours. After the mixture was cooled to room temperature, the reaction mixture was diluted with 40 mL of distilled water and an aqueous phase was extracted with CH₂Cl₂. An organic layer was dried with anhydrous Na₂SO₄, and a solvent was evaporated using a rotary evaporator. Compounds 4a to 4d were separated as an oily residue using vacuum distillation.

<2-2> Glycosylation Reaction and de-O-benzoylation Reaction Under Zemplen Conditions (Synthesis of Compounds 5a to 5d)

Compounds 5a to 5d were prepared from compounds 4a to 4c using the same method as described in Example 1-3.

<2-3> Glycosylation Reaction (Synthesis of PSE-C7a to PSE-C13a)

PSE-C7a to PSE-C13a were prepared from compounds 5a to 5d using the same method as described in Example 1-4.

<2-4> De-O-benzoylation Reaction Under Zemplen Conditions (Synthesis of PSE-C7 to PSE-C13)

PSE-C7 to PSE-C13 were prepared from PSE-C7a to PSE-C13a using the same method as described in Example 1-5.

### PREPARATION EXAMPLE 4

### Synthesis of PSE-C7

<4-1> Synthesis of 1,3-bis (heptyloxy)propan-2-ol (Compound 4a)

According to the general synthetic procedures for alcohol derivatives described in Example 1-1, 1,3-bis (heptyloxy)propan-2-ol (compound 4a) was prepared in a yield of 80% using 1-nonanol (1-haptanol) as an alcohol. ¹H NMR (400 MHz, CDCl₃): δ 3.95-3.92 (m, 1H), 3.46-3.43 (m, 8H), 2.47 (d, J=4.0 Hz, 1H), 1.57-1.54 (m, 4H), 1.30-1.26 (m, 16H), 0.87 (t, J=8.0 Hz, 6H); ¹³C NMR (400 MHz, CDCl₃): δ 71.9, 71.7, 69.5, 32.1, 29.7, 29.6, 29.5, 26.2, 22.7, 14.3.

<4-2> Synthesis of Compound 5a

According to the general procedures for glycosylation and de-O-benzoylation described in Example 2-2, compound 5a was synthesized. Yield: 85%; ¹H NMR (400 MHz, CD₃OD): δ 4.45 (d, J=8.0 Hz, 1H), 4.01 (quint, J=8.0 Hz 1H), 3.87-3.82 (m, 1H), 3.60-3.56 (m, 4H), 3.52-3.46 (m, 4H), 3.35-3.20 (m, 4H), 3.18 (t, J=8.0 Hz, 1H), 1.60-1.55 (m, 4H), 1.40-1.25 (m, 16H), 0.90 (t, J=8.0 Hz, 6H); ¹³C NMR (400 MHz, CD₃OD): δ 104.1, 103.9, 80.1, 78.4, 78.2, 77.9, 75.2, 72.7, 71.9, 71.8, 71.5, 71.3, 71.1, 62.9, 33.2, 30.9, 30.8, 30.4, 27.4, 27.3, 23.8, 14.6.

<4-3> Synthesis of PSE-C7a

According to the general procedures for glycosylation described in Example 2-3, PSE-C7a was synthesized. Yield: 80%; ¹H NMR (400 MHz, CDCl₃): δ 8.24 (d, J=8.0 Hz, 2H), 8.16-7.98 (m, 26H), 7.80-7.70 (m, 4H), 7.60-7.52 (m, 2H), 7.50-7.18 (m, 46H), 5.92-5.81 (m, 3H), 5.73-5.61 (m, 5H), 5.58-5.52 (m, 2H), 5.44 (m, 2H), 5.07 (d, J=7.6 Hz, 2H), 4.96-4.71 (m, 3H), 4.69 (d, J=3.2 Hz, 1H), 4.65-4.58 (m, 3H), 4.52-4.39 (m, 4H), 4.23-3.96 (m, 6H), 3.83-3.76 (m, 3H), 3.68 (t, J=8.0 Hz, 1H), 3.52-3.23 (m, 8H), 3.15 (br s, 1H), 3.01 (br s, 1H), 1.71-1.62 (m, 2H), 1.49-1.42 (m, 2H), 1.40-1.18 (m, 16H), 0.84 (t, J=8.0 Hz, 6H); ¹³C NMR (400 MHz, CDCl₃): δ 166.2, 166.0, 165.8, 165.7, 165.2, 165.1, 164.6, 164.5, 133.6, 133.4, 133.3, 133.1, 132.9, 130.2, 129.9, 129.4, 129.3, 129.2, 129.1, 129.0, 128.6, 128.5, 128.4, 128.3, 128.2, 101.6, 100.5, 99.5, 99.3, 74.9, 73.4, 72.9, 72.8, 72.5, 72.2, 71.9, 71.2, 71.7, 71.6, 70.7, 70.3, 69.4, 63.8, 63.6, 62.9, 60.4, 53.6, 32.0, 29.9, 29.8, 29.7, 29.5, 26.2, 22.8, 14.3, 14.1.

<4-4> Synthesis of PSE-C7

According to the general procedures for de-O-benzoylation described in Example 2-4, PSE-C7 was synthesized. Yield: 92%; ¹H NMR (400 MHz, CD₃OD): δ 4.95 (d, J=8.0 Hz, 1H), 4.76 (d, J=7.6 Hz, 1H), 4.71 (d, J=7.2 Hz, 1H), 4.66 (d, J=7.6 Hz, 1H), 4.38 (d, J=8.0 Hz, 1H), 4.26 (d, J=9.6 Hz, 1H), 4.08 (t, J=8.0 Hz, 1H), 4.39-3.80 (m, 8H), 3.72-3.55 (m, 8H), 3.50-3.19 (m, 20H), 1.58 (m, 4H), 1.39-1.20 (m, 16H), 0.90 (t, J=8.0 Hz, 6H); ¹³C NMR (400 MHz, CD₃OD): δ 104.9, 103.7, 103.1, 102.6, 102.4, 81.8, 79.7, 78.2, 78.1, 78.0, 77.9, 77.8, 76.0, 75.9, 75.2, 75.0, 72.8, 72.7, 71.8, 71.7, 71.6, 71.5, 71.1, 69.8, 63.0, 62.9, 62.6, 33.2, 30.9, 30.8, 30.4, 27.4, 23.8, 14.6.

### PREPARATION EXAMPLE 5

### Synthesis of PSE-C9

<5-1> Synthesis of 1,3-bis (nonyloxy)propan-2-ol (Compound 4b)

According to the general synthetic procedures for an alcohol derivative described in Example 1-1, 1,3-bis (nonyloxy)propan-2-ol (compound 4b) was prepared in a yield of 77% using 1-nonanol as an alcohol. ¹H NMR (400 MHz, CDCl₃): δ 3.95-3.93 (m, 1H), 3.47-3.43 (m, 8H), 2.47 (d, J=4.0 Hz, 1H), 1.57-1.54 (m, 4H), 1.30-1.26 (m, 24H), 0.88 (t, J=8.0 Hz, 6H); ¹³C NMR (100 MHz, CDCl₃): δ 71.9, 71.7, 70.0, 32.1, 29.7, 29.6, 29.5, 29.4, 26.2, 22.7, 14.3.

<5-2> Synthesis of Compound 5b

According to the general procedures for glycosylation and de-O-benzoylation described in Example 2-2, compound 5b was synthesized. Yield: 86%; ¹H NMR (400 MHz, CD₃OD): δ 4.45 (d, J=8.0 Hz, 1H), 4.01 (quint, J=8.0 Hz 1H), 3.87-3.82 (m, 1H), 3.60-3.56 (m, 4H), 3.52-3.46 (m, 4H), 3.36-3.20 (m, 4H), 3.18 (t, J=8.0 Hz, 1H), 1.60-1.55 (m, 4H), 1.42-1.24 (m, 24H), 0.90 (t, J=8.0 Hz, 6H); ¹³C NMR (100 MHz, CD₃OD): δ 104.1, 103.9, 80.1, 78.4, 78.2, 77.9, 75.2, 72.7, 71.9, 71.8, 71.5, 71.3, 71.1, 62.9, 33.2, 30.9, 30.4, 27.4, 27.3, 23.8, 14.6.

<5-3> Synthesis of PSE-C9a

According to the general procedures for glycosylation described in Example 2-3, PSE-C9a was synthesized. Yield: 82%; ¹H NMR (400 MHz, CDCl₃): δ 8.24 (d, J=8.0 Hz, 2H), 8.16-7.98 (m, 26H), 7.80-7.70 (m, 4H), 7.60-7.52 (m, 2H), 7.50-7.18 (m, 46H), 5.91-5.81 (m, 3H), 5.73-5.61 (m, 5H), 5.58-5.52 (m, 2H), 5.48-5.44 (m, 2H), 5.07 (d, J=7.6 Hz, 2H), 4.96-4.71 (m, 3H), 4.69 (d, J=3.2 Hz, 1H), 4.65-4.56 (m, 3H), 4.52-4.39 (m, 4H), 4.23-3.96 (m, 6H), 3.83-3.76 (m, 3H), 3.68 (t, J=8.0 Hz, 1H), 3.52-3.23 (m, 8H), 3.15 (br s, 1H), 3.01 (br s, 1H), 1.71-1.62 (m, 2H), 1.49-1.42 (m, 2H), 1.40-1.17 (m, 24H), 0.84 (t, J=8.0 Hz, 6H); ¹³C NMR (100 MHz, CDCl₃): δ 166.2, 166.0, 165.8, 165.7, 165.2, 165.1, 164.6, 164.5, 133.6, 133.4, 133.3, 133.1, 132.9, 130.2, 129.9, 129.4, 129.3, 129.2, 129.1, 129.0, 128.6, 128.5, 128.4, 128.3, 128.2, 101.6, 100.5, 99.5, 99.3, 74.9, 73.4, 72.9, 72.8, 72.5, 72.2, 71.9, 71.7, 71.6, 71.2, 70.7, 70.3, 69.4, 63.8, 63.6, 62.9, 60.4, 53.6, 32.0, 29.9, 29.8, 29.7, 29.5, 26.2, 22.8, 14.3, 14.1.

<5-4> Synthesis of PSE-C9

According to the general procedures for de-O-benzoylation described in Example 2-4, PSE-C9 was synthesized. Yield: 92%; ¹H NMR (400 MHz, CD₃OD): δ 4.95 (d, J=8.0 Hz, 1H), 4.76 (d, J=8.0 Hz, 1H), 4.71 (d, J=7.2 Hz, 1H), 4.66 (d, J=8.0 Hz, 1H), 4.38 (d, J=8.0 Hz, 1H), 4.26 (d, J=9.6 Hz, 1H), 4.01 (t, J=8.0 Hz, 1H), 4.99-3.80 (m, 8H), 3.72-3.55 (m, 8H), 3.51-3.19 (m, 20H), 1.58 (m, 4H), 1.39-1.20 (m, 24H), 0.90 (t, J=8.0 Hz, 6H); ¹³C NMR (100 MHz, CD₃OD): δ 104.8, 103.7, 103.1, 102.6, 102.4, 81.8, 79.7, 78.2, 78.1, 78.0, 77.9, 77.8, 76.0, 75.9, 75.2, 75.0, 72.8, 72.7, 71.8, 71.7, 71.6, 71.5, 71.1, 69.8, 63.0, 62.9, 62.6, 33.2, 30.9, 30.8, 30.7, 30.6, 27.4, 23.8, 14.6; HRMS (EI): calcd. for C₅₁H₉₄O₂₈ [M+Na]⁺ 1177.5829, found 1177.5833.

### PREPARATION EXAMPLE 6

### Synthesis of PSE-C11

<6-1> Synthesis of 1,3-bis (undecyloxy)propan-2-ol (Compound 4c)

According to the general synthetic procedures for an alcohol derivative described in Example 1-1, 1,3-bis (undecyloxy)propan-2-ol (compound 4c) was prepared in a yield of 77% using 1-undecanol as an alcohol. ¹H NMR (400 MHz, CDCl₃): δ 3.95-3.93 (m, 1H), 3.43-3.40 (m, 8H), 2.47 (d, J=4.0 Hz, 1H), 1.57-1.54 (m, 4H), 1.32-1.25 (m, 32H), 0.88 (t, J=8.0 Hz, 6H); ¹³C NMR (100 MHz, CDCl₃): δ 71.9, 71.7, 69.5, 31.9, 29.7, 29.5, 29.4, 26.1, 22.7, 14.3.

<6-2> Synthesis of Compound 5c

According to the general procedures for glycosylation and de-O-benzoylation described in Example 2-2, compound 5c was synthesized. Yield: 86%; ¹H NMR (400 MHz, CD₃OD): δ 4.46 (d, J=8.0 Hz, 1H), 3.99 (quint, J=8.0 Hz 1H), 3.88-3.81 (m, 1H), 3.65-3.56 (m, 4H), 3.52-3.46 (m, 4H), 3.36-3.20 (m, 4H), 3.18 (t, J=8.0 Hz, 1H), 1.60-1.55 (m, 4H), 1.42-1.24 (m, 32H), 0.90 (t, J=8.0 Hz, 6H); ¹³C NMR (100 MHz, CD₃OD): δ 104.0, 103.9, 80.1, 79.9, 78.3, 78.2, 77.8, 75.1, 72.7, 71.9, 71.8, 71.5, 71.3, 71.1, 62.8, 33.2, 31.0, 30.9, 30.8, 30.4, 27.4, 27.3, 23.8, 14.7.

<6-3> Synthesis of PSE-C11a

According to the general procedures for glycosylation described in Example 2-3, PSE-C11a was synthesized. Yield: 82%; ¹H NMR (400 MHz, CDCl₃): δ 8.24 (d, J=8.0 Hz, 2H), 8.16-7.98 (m, 26H), 7.80-7.70 (m, 4H), 7.60-7.52 (m, 2H), 7.50-7.18 (m, 46H), 5.92-5.81 (m, 3H), 5.73-5.61 (m, 5H), 5.58-5.52 (m, 2H), 5.48-5.44 (m, 2H), 5.07 (d, J=7.6 Hz, 2H), 4.96-4.71 (m, 3H), 4.69 (d, J=3.2 Hz, 1H), 4.65-4.58 (m, 3H), 4.52-4.39 (m, 4H), 4.23-3.95 (m, 6H), 3.83-3.76 (m, 3H), 3.68 (t, J=8.0 Hz, 1H), 3.52-3.23 (m, 8H), 3.09 (br s, 1H), 3.01 (br s, 1H), 1.71-1.62 (m, 2H), 1.49-1.42 (m, 2H), 1.41-1.18 (m, 32H), 0.84 (t, J=8.0 Hz, 6H); ¹³C NMR (100 MHz, CDCl₃): δ 166.2, 166.0, 165.8, 165.7, 165.2, 165.1, 164.6, 164.5, 133.6, 133.4, 133.3, 133.1, 132.9, 130.2, 129.9, 129.4, 129.3, 129.2, 129.1, 129.0, 128.6, 128.5, 128.4, 128.3, 128.2, 101.6, 100.5, 99.5, 99.3, 74.9, 73.4, 72.9, 72.8, 72.5, 71.9, 71.2, 71.7, 71.6, 71.2, 70.7, 70.3, 69.4, 63.8, 63.6, 62.9, 60.4, 53.6, 32.0, 29.9, 29.8, 29.7, 29.5, 26.3, 26.2, 22.8, 14.3, 14.1.

<6-4> Synthesis of PSE-C11

According to the general procedures for de-O-benzoylation described in Example 2-4, PSE-C11 was synthesized. Yield: 92%; ¹H NMR (400 MHz, CD₃OD): δ 4.97 (d, J=7.6 Hz, 1H), 4.77 (d, J=8.0 Hz, 1H), 4.70 (d, J=7.2 Hz, 1H), 4.67 (d, J=7.6 Hz, 1H), 4.38 (d, J=8.0 Hz, 1H), 4.26 (d, J=10.4 Hz, 1H), 4.09 (t, J=8.4 Hz, 1H), 4.00-3.68 (m, 8H), 3.70-3.55 (m, 8H), 3.50-3.20 (m, 20H), 1.57 (m, 4H), 1.39-1.20 (m, 32H), 0.89 (t, J=8.0 Hz, 6H); ¹³C NMR (100 MHz, CD₃OD): δ 104.8, 103.6, 103.1, 102.6, 102.4, 81.8, 79.7, 78.2, 78.1, 78.0, 77.9, 77.8, 76.0, 75.9, 75.2, 75.0, 72.8, 72.7, 71.8, 71.7, 71.6, 71.5, 71.1, 69.8, 63.0, 62.9, 62.6, 33.2, 30.9, 30.9, 30.8, 30.7, 30.6, 27.4, 23.8, 14.6; HRMS (EI): calcd. for C₅₅H₁₀₂O₂₈ [M+Na]⁺ 1233.6455, found 1233.6451.

### PREPARATION EXAMPLE 7

### Synthesis of PSE-C13

<7-1> Synthesis of 1,3-bis (tridecyloxy)propan-2-ol (Compound 4d)

According to the general synthetic procedures for an alcohol derivative described in Example 1-1, 1,3-bis (tridecyloxy)propan-2-ol (compound 4d) was prepared in a yield of 75% using 1-tridecanol as an alcohol. ¹H NMR (400 MHz, CDCl₃): δ 3.95-3.92 (m, 1H), 3.45-3.40 (m, 8H), 2.47 (d, J=4.0 Hz, 1H), 1.57-1.54 (m, 4H), 1.32-1.25 (m, 40H), 0.88 (t, J=8.0 Hz, 6H); ¹³C NMR (100 MHz, CDCl₃): δ 71.9, 71.7, 70.0, 32.1, 29.7, 29.6, 29.5, 29.4, 26.2, 22.9, 14.3.

<7-2> Synthesis of Compound 5d

According to the general procedures for glycosylation and de-O-benzoylation described in Example 2-2, compound 5d was synthesized. Yield: 85%; ¹H NMR (400 MHz, CD₃OD): δ 4.46 (d, J=8.0 Hz, 1H), 3.99 (quint, J=8.0 Hz 1H), 3.87-3.81 (m, 1H), 3.65-3.56 (m, 4H), 3.52-3.46 (m, 4H), 3.36-3.20 (m, 4H), 3.18 (t, J=8.0 Hz, 1H), 1.60-1.55 (m, 4H), 1.42-1.24 (m, 40H), 0.89 (t, J=8.0 Hz, 6H); ¹³C NMR (100 MHz, CD₃OD): δ 104.0, 103.9, 80.1, 79.9, 78.3, 78.2, 77.8, 75.1, 72.7, 71.9, 71.8, 71.5, 71.3, 71.1, 62.8, 33.2, 31.0, 30.9, 30.8, 30.7, 27.5, 27.4, 27.3, 23.8, 14.7.

<7-3> Synthesis of PSE-C13a

According to the general procedures for glycosylation described in Example 2-3, PSE-C13a was synthesized. Yield: 80%; ¹H NMR (400 MHz, CDCl₃): δ 8.24 (d, J=8.0 Hz, 2H), 8.16-7.98 (m, 26H), 7.80-7.70 (m, 4H), 7.60-7.52 (m, 2H), 7.50-7.18 (m, 46H), 5.92-5.81 (m, 3H), 5.73-5.61 (m, 5H), 5.58-5.52 (m, 2H), 5.48-5.44 (m, 2H), 5.07 (d, J=7.6 Hz, 2H), 4.96-4.70 (m, 3H), 4.69 (d, J=3.2 Hz, 1H), 4.65-4.58 (m, 3H), 4.52-4.39 (m, 4H), 4.23-3.96 (m, 6H), 3.83-3.76 (m, 3H), 3.68 (t, J=8.0 Hz, 1H), 3.52-3.23 (m, 8H), 3.09 (br s, 1H), 3.01 (br s, 1H), 1.71-1.62 (m, 2H), 1.49-1.42 (m, 2H), 1.41-1.18 (m, 40H), 0.84 (t, J=8.0 Hz, 6H); ¹³C NMR (100 MHz, CDCl₃): δ 166.2, 166.0, 165.8, 165.7, 165.2, 165.2, 165.1, 165.1, 164.6, 164.5, 133.6, 133.4, 133.3, 133.1, 132.9, 130.2, 129.9, 129.4, 129.3, 129.2, 129.1, 129.0, 128.6, 128.5, 128.4, 128.3, 128.2, 101.6, 100.5, 99.5, 99.3, 74.9, 73.4, 72.9, 72.8, 72.5, 71.9, 71.2, 71.7, 71.6, 71.2, 70.7, 70.3, 69.4, 63.8, 63.6, 62.9, 60.4, 53.6, 32.0, 29.9, 29.8, 29.7, 29.5, 26.3, 26.2, 22.8, 14.3. HRMS (EI): calcd. for C₅₉H₁₁₀O₂₈ [M+Na]⁺ 1289.7081, found 1289.7078.

<7-4> Synthesis of PSE-C13

According to the general procedures for de-O-benzoylation described in Example 2-4, PSE-C13 was synthesized. Yield: 90%; ¹H NMR (400 MHz, CD₃OD): δ 4.95 (d, J=7.6 Hz, 1H), 4.76 (d, J=7.6 Hz, 1H), 4.71 (d, J=7.2 Hz, 1H), 4.66 (d, J=7.6 Hz, 1H), 4.38 (d, J=8.0 Hz, 1H), 4.26 (d, J=9.6 Hz, 1H), 4.08 (t, J=8.0 Hz, 1H), 4.03-3.80 (m, 8H), 3.72-3.55 (m, 8H), 3.50-3.19 (m, 20H), 1.58 (m, 4H), 1.39-1.20 (m, 40H), 0.89 (t, J=8.0 Hz, 6H); ¹³C NMR (100 MHz, CD₃OD): δ 104.9, 103.6, 103.1, 102.6, 102.4, 81.8, 79.7, 78.2, 78.1, 78.0, 77.9, 77.8, 76.0, 75.9, 75.2, 75.0, 72.8, 72.7, 71.8, 71.7, 71.6, 71.5, 71.1, 69.8, 63.0, 62.9, 62.6, 33.2, 31.1, 30.9, 30.8, 30.7, 30.6, 27.5, 23.9, 14.6.

### EXAMPLE 3

### Methods of Synthesizing TPS-Es and TPS-ELs

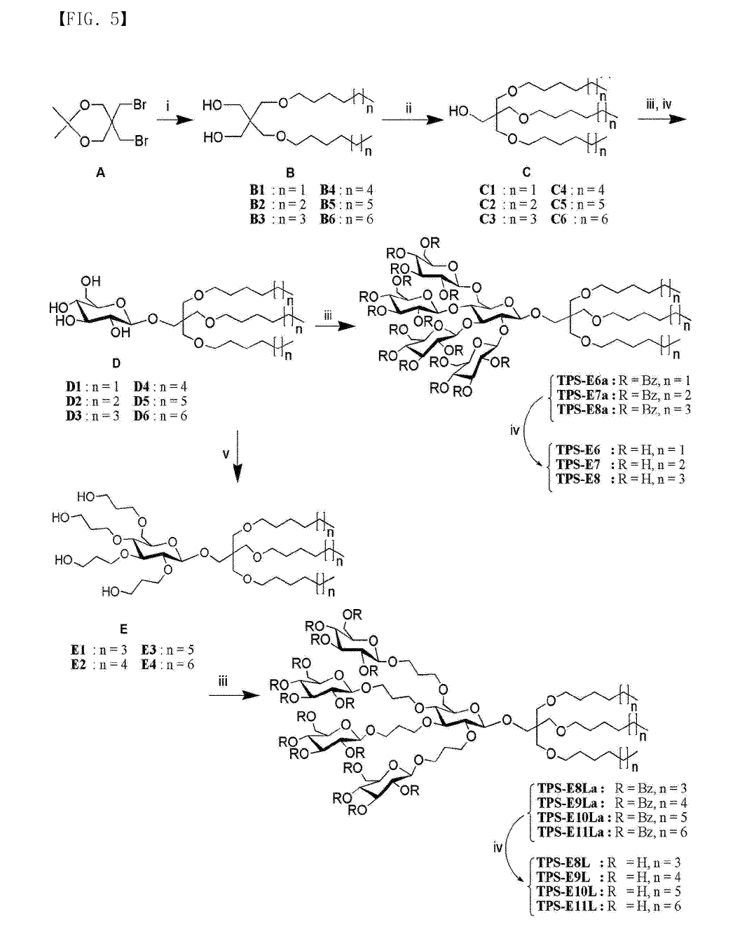

A synthetic scheme for TPS-Es is shown in FIG. 5. Seven kinds of tripod penta-saccharide amphiphiles (TPS-Es and TPS-ELs) were synthesized according to methods described in the following <3-1> to <3-4>.

<3-1> General Synthetic Procedures for dialkylated diol (Synthesis of Compound B, Step i in FIG. 5)

In an anhydrous two-neck flask, a solution, in which NaH (0.17 mmol) was dissolved in DMF, was treated with an alcohol (17 mmol) at 0° C. under a N₂ atmosphere and was stirred at room temperature for 30 minutes. After adding 5,5-bis-(bromomethyl)-2,2-dimethyl-[1,3] dioxane (compound A) (4.3 mmol) to the reaction mixture, the mixture was maintained at 120° C. for 15 hours. After cooling to room temperature, the reaction mixture was treated with ice cold water to terminate the reaction, and extraction was performed three times using diethyl ether. A mixed organic layer was washed with brine, dried using anhydrous Na₂SO₄, and concentrated using a rotary evaporator. After complete evaporation, p-toluenesulfonic acid (p-TSA) monohydrate (catalytic amount) was added to residues dissolved in a mixture of CH₂Cl₂ and MeOH in a ratio of 1:1, and the mixture was stirred at room temperature for 2 hours. The reaction mixture was neutralized with a saturated aqueous NaHCO₃ solution, and the volume of the solvent was reduced using a rotary evaporator. The reaction mixture was layered between CH₂Cl₂ and H₂O. The separated organic layer was washed with brine, dried using anhydrous Na₂SO₄, and concentrated in vacuo. An ether containing a diol (B) was obtained in the form of a white solid (92-94% (two steps)) using flash column chromatography (EtOAc/hexane).

<3-2> General Synthetic Procedures for trialkylated mono-ol (Synthesis of Compound C, Step ii in FIG. 5)

In a two-neck flask filled with argon under anhydrous conditions, a solution, in which NaH (212.0 mmol) was stirred in dry DMF, was treated with a solution, in which a diol derivative (compound B) (212.0 mmol) was dissolved in dry DMF. After 20 minutes, the mixture was treated with a 1-bromoalkane (RBr) (330.0 mmol) and heated to 100° C. The reaction mixture was left at the same temperature for 4 hours, then cooled to room temperature, and the reaction was terminated with H₂O. The reaction mixture was extracted twice using CH₂Cl₂, washed with brine, and dried using anhydrous Na₂SO₄. The reaction mixture was purified using silica-gel column chromatography (EtOAc/hexane) to obtain a trialkyl-containing mono-ol (compound C) as an oily liquid (yield of 85 to 90%).

<3-3> Glycosylation Reaction and General Procedures for de-O-benzoylation Reaction Under Zemplen Conditions (Synthesis of Compound D, Steps iii and iv in FIG. 5)

The method used to carry out this reaction is a modification of the method described in the journal article (P.S. Chae et al., Chem. Eur. J. 2013, 19, 15645-15651) published by the present inventors.

Briefly, a mixture of a mono-ol derivative (compound C) dissolved in anhydrous CH₂Cl₂ (30 mL), AgOTf (1.2 or 4.5 equiv.) and 2,4,6-collidine (0.7 or 2.0 equiv.) was stirred at −45° C. Next, perbenzoylated glucosylbromide (1.2 or 4.5 equiv.) dissolved in CH₂Cl₂ (30 mL) was transferred via a cannula to the solution over 30 minutes. The reaction product was allowed to warm to 0° C. for 1.5 hours. The progress of the reaction was monitored by TLC. After completion of the reaction (as determined by TLC), pyridine was added to the reaction mixture. The reaction mixture was diluted with CH₂Cl₂ and filtered through Celite. The filtrate was washed successively with a 1M Na₂S₂O₃ aqueous solution, 0.1M HCl aqueous solution and brine. Then, an organic layer was dried with anhydrous Na₂SO₄, and the solvent was removed using a rotary evaporator. Glycosylated residues were dissolved in MeOH and then a methanolic solution of 0.5 M NaOMe was added in a required amount so that the final concentration of NaOMe was 0.05 M. The reaction mixture was stirred at room temperature for 6 hours and then neutralized with Amberlite IR-120 (H⁺ form) resin. The resin was removed by filtration, washed with MeOH, and then the solvent was removed from the filtrate in vacuo. The residues were purified using silica-gel column chromatography (MeOH/CH₂Cl₂) to obtain a product (compound D) in the form of a white solid (yield of 88 to 90% (two steps)).

<3-4> General Procedures for Allylation and Hydroboration (Synthesis of Compound E, Step v in FIG. 5)

NaH (99 mmol) and ally! bromide (96 mmol) were added to a suspension containing compounds D3 to D6 (13.1 mmol) dissolved in dry DMF (100 mL), and the reaction mixture was stirred at room temperature overnight. The reaction was terminated with ice water at 0° C. and CH₂Cl₂ (30 mL) was added thereto. An organic phase was separated, washed successively with water (3×) and brine (2×), dried (Na₂SO₄), filtered, concentrated using a rotary evaporator, and dried in a high vacuum overnight. Then, a 0.5 M 9-BBN-H solution dissolved in THF (28 mL, 14 mmol) was added to a solution of an allylated product (1.55 mmol) dissolved in dry THF (30 mL), and the reaction mixture was stirred at room temperature for 1.5 hours. Excess reagents were removed by adding ice water. Next, 3 M aqueous NaOH (14 mL) and 30% H₂O₂ (14 mL) were added to the mixture slowly at the same time to oxidize the mixture and the mixture was stirred at room temperature overnight. The mixture was saturated with K₂CO₃ and layers were separated. An aqueous layer was washed with EtOAc (3×), and a mixed organic phase was concentrated and purified using flash chromatography to obtain a desired product (compound E; yield of 75-80% (two steps)).

### PREPARATION EXAMPLE 8

### Synthesis of TPS-E6

<8-1> Synthesis of 2,2-bis ((hexyloxy)methyl)propane-1,3-diol (Compound B1)

According to the general synthetic procedures for a dialkylated diol described in Example 3-1, 2,2-bis ((hexyloxy)methyl)propane-1,3-diol was prepared in a yield of 92%. ¹H NMR (400 MHz, CDCl₃): δ 3.65 (d, J=4.0 Hz, 4H), 3.51 (s, 4H), 3.42 (t, J=4.0 Hz, 4H), 2.85 (t, J=4.0 Hz, 2H), 1.56 (quin, J=4.0 Hz, 4H), 1.38-1.21 (m, 12H), 0.87 (t, J=8.0 Hz, 6H); ¹³C NMR (100 MHz, CDCl₃): δ 72.1, 71.8, 44.7, 31.5, 29.4, 25.7, 22.5, 14.0.

<8-2> Synthesis of 3-(hexyloxy)-2,2-bis ((hexyloxy)methyl)propan-1-ol (Compound C1)

According to the general synthetic procedures for a trialkylated mono-ol described in Example 3-2, 3-(hexyloxy)-2,2-bis ((hexyloxy)methyl)propan-1-ol was prepared in a yield of 87%. ¹H NMR (400 MHz, CDCl₃): δ 3.72 (d, J=8.0 Hz, 2H), 3.44 (s, 6H), 3.39 (t, J=8.0 Hz, 6H), 3.17 (t, J=4.0 Hz, 1H), 1.54 (quin, J=4.0 Hz, 6H), 1.40-1.21 (m, 18H), 0.87 (t, J=8.0 Hz, 9H); ¹³C NMR (100 MHz, CDCl₃): δ 71.6, 71.3, 66.3, 44.7, 31.6, 29.5, 25.8, 22.6, 14.1.

<8-3> Synthesis of Compound D1

According to the general synthetic procedures for glycosylation and de-O-benzoylation described in Example 3-3, compound D1 was prepared in a yield of 88%. ¹H NMR (400 MHz, CD₃OD): δ 4.21 (d, J=8.0 Hz, 1H), 3.85 (quint, J=8.0 Hz, 2H), 3.70-3.65 (m, 1H), 3.51 (d, J=8.0 Hz, 1H), 3.45-3.36 (m, 12H), 3.32-3.28 (m, 2H), 3.26-3.15 (m, 2H), 1.59-1.50 (m, 6H), 1.40-1.22 (m, 18H), 0.90 (t, J=8.0 Hz, 9H); ¹³C NMR (100 MHz, CD₃OD): δ 105.5, 78.1, 77.9, 75.2, 72.6, 71.6, 70.6, 70.5, 62.8, 46.7, 33.0, 30.8, 27.2, 23.8, 14.6.

<8-4> Synthesis of TPS-E6a

According to the general synthetic procedures for glycosylation described in Example 3-3, TPS-E6a was prepared in a yield of 80%. ¹H NMR (400 MHz, CDCl₃): δ 8.24 (d, J=8.0 Hz, 2H), 8.15-7.78 (m, 24H), 7.72-7.67 (m, 5H), 7.66-7.59 (m, 3H), 7.58-7.12 (m, 46H), 5.96 (t, J=8.0 Hz, 1H), 5.92-5.82 (m, 3H), 5.71-5.69 (m, 2H), 5.61-5.42 (m, 6H), 5.01 (d, J=8.0 Hz, 1H), 4.98-4.92 (m, 2H), 4.88-4.79 (m, 2H), 4.77-4.65 (m, 2H), 4.58-4.42 (m, 3H), 4.36-4.29 (m, 1H), 4.26-4.13 (m, 2H), 4.12-4.05 (m, 2H), 4.03-3.95 (d, J=8.0 Hz, 1H), 3.91-3.71 (m, 4H), 3.37-3.18 (m, 14H), 2.85 (br s, 1H), 2.75 (br s, 1H), 1.59-1.45 (m, 6H), 1.38-1.19 (m, 18H), 0.84 (t, J=8.0 Hz, 9H); ¹³C NMR (100 MHz, CDCl₃): δ 166.2, 166.1, 166.0, 165.9, 165.8, 165.6, 165.2, 165.1, 164.9, 164.4, 133.4, 133.1, 130.1, 129.9, 129.8, 129.7, 129.5, 129.4, 129.3, 129.2, 129.1, 129.0, 128.9, 128.6, 128.5, 128.4, 128.3, 128.2, 101.7, 101.5, 99.9, 99.8, 74.9, 73.4, 72.8, 72.7, 72.6, 72.5, 72.4, 71.6, 71.3, 70.6, 70.3, 70.2, 69.4, 68.8, 63.7, 63.5, 45.6, 31.8, 29.8, 26.0, 22.8, 14.2.

<8-5> Synthesis of TPS-E6

According to the general synthetic procedures for de-O-benzoylation described in Example 3-3, TPS-E6 was prepared in a yield of 94%. ¹H NMR (400 MHz, CD₃OD): δ 4.95 (d, J=8.0 Hz, 1H), 4.85 (d, J=8.0 Hz, 1H), 4.66 (d, J=8.0 Hz, 1H), 4.38 (t, J=8.0 Hz, 2H), 4.25 (d, J=10.4 Hz, 1H), 4.12-3.95 (m, 3H), 3.90-3.82 (m, 6H), 3.75-3.59 (m, 6H), 3.46-3.18 (m, 28H), 1.60-1.51 (m, 6H), 1.40-1.28 (m, 18H), 0.89 (t, J=8.0 Hz, 9H); ¹³C NMR (100 MHz, CD₃OD): δ 104.7, 104.0, 103.4, 102.9, 102.6, 80.3, 80.2, 78.4, 78.0, 77.9, 77.8, 77.6, 76.0, 75.4, 75.2, 75.1, 72.5, 72.1, 71.7, 71.5, 71.3, 70.8, 70.2, 69.2, 63.3, 62.9, 62.7, 62.5, 46.8, 32.9, 30.8, 27.2, 23.8, 14.6; HRMS (EI): calcd. for C₅₃H₉₈O₂₉ [M+Na]⁺ 1221.6091, observed 1221.6095.

### PREPARATION EXAMPLE 0

### Synthesis of TPS-E7

<9-1> Synthesis of 2,2-bis ((heptyloxy)methyl)propane-1,3-diol (Compound B2)

According to the general synthetic procedures for a dialkylated diol described in Example 3-1, 2,2-bis ((heptyloxy)methyl)propane-1,3-diol was prepared in a yield of 92%. ¹H NMR (400 MHz, CDCl₃): δ 3.65 (d, J=4.0 Hz, 4H), 3.51 (s, 4H), 3.42 (t, J=4.0 Hz, 4H), 2.85 (t, J=4.0 Hz, 2H), 1.56 (quin, J=4.0 Hz, 4H), 1.38-1.21 (m, 16H), 0.87 (t, J=8.0 Hz, 6H); ¹³C NMR (100 MHz, CDCl₃): δ 72.2, 71.8, 44.7, 31.9, 29.6, 29.4, 26.3, 22.6, 14.0.

<9-2> Synthesis of 3-(heptyloxy)-2,2-bis ((heptyloxy)methyl)propan-1-ol (Compound C2)

According to the general synthetic procedures for a trialkylated mono-ol described in Example 3-2, 3-(heptyloxy)-2,2-bis ((heptyloxy)methyl)propan-1-ol was prepared in a yield of 90%. ¹H NMR (400 MHz, CDCl₃): δ 3.72 (d, J=8.0 Hz, 2H), 3.44 (s, 6H), 3.39 (t, J=8.0 Hz, 6H), 3.17 (t, J=4.0 Hz, 1H), 1.54 (quin, J=4.0 Hz, 6H), 1.40-1.21 (m, 18H), 0.87 (t, J=8.0 Hz, 9H); ¹³C NMR (100 MHz, CDCl₃): δ 71.6, 71.3, 66.3, 44.7, 31.6, 29.5, 25.8, 22.6, 14.1.

<9-3> Synthesis of Compound D2

According to the general synthetic procedures for glycosylation and de-O-benzoylation described in Example 3-3, compound D2 was prepared in a yield of 89%. ¹H NMR (400 MHz, CD₃OD): δ 4.21 (d, J=8.0 Hz, 1H), 3.85 (quint, J=8.0 Hz, 2H), 3.70-3.65 (m, 1H), 3.51 (d, J=8.0 Hz, 1H), 3.45-3.36 (m, 12H), 3.32-3.28 (m, 2H), 3.26-3.15 (m, 2H), 1.59-1.50 (m, 6H), 1.40-1.22 (m, 24H), 0.90 (t, J=8.0 Hz, 9H); ¹³C NMR (100 MHz, CD₃OD): δ 105.5, 78.1, 77.9, 75.2, 72.6, 71.6, 70.6, 70.5, 62.8, 46.7, 33.2, 30.9, 30.4, 27.5, 23.8, 14.7.

<9-4> Synthesis of TPS-E7a

According to the general synthetic procedures for glycosylation described in Example 3-3, TPS-E7a was prepared in a yield of 78%. ¹H NMR (400 MHz, CDCl₃): δ 8.24 (d, J=8.0 Hz, 2H), 8.15-7.78 (m, 24H), 7.72-7.67 (m, 5H), 7.66-7.59 (m, 3H), 7.58-7.12 (m, 46H), 5.96 (t, J=8.0 Hz, 1H), 5.92-5.82 (m, 3H), 5.71-5.69 (m, 2H), 5.61-5.42 (m, 6H), 5.01 (d, J=8.0 Hz, 1H), 4.98-4.91 (m, 2H), 4.88-4.79 (m, 2H), 4.77-4.65 (m, 2H), 4.59-4.42 (m, 3H), 4.36-4.29 (m, 1H), 4.26-4.13 (m, 2H), 4.12-4.05 (m, 2H), 4.03-3.95 (d, J=8.0 Hz, 1H), 3.91-3.71 (m, 4H), 3.37-3.18 (m, 14H), 2.85 (br s, 1H), 2.75 (br s, 1H), 1.59-1.45 (m, 6H), 1.40-1.18 (m, 24H), 0.84 (t, J=8.0 Hz, 9H); ¹³C NMR (100 MHz, CDCl₃): δ 166.2, 166.1, 166.0, 165.9, 165.8, 165.6, 165.2, 165.1, 164.9, 164.4, 133.4, 133.1, 130.1, 129.9, 129.8, 129.7, 129.5, 129.4, 129.3, 129.2, 129.1, 129.0, 128.9, 128.6, 128.5, 128.4, 128.3, 128.2, 101.7, 101.5, 99.9, 99.8, 74.9, 73.4, 72.8, 72.7, 72.6, 72.5, 72.4, 71.6, 71.3, 70.6, 70.3, 70.2, 69.4, 68.8, 63.7, 63.5, 45.6, 32.1, 29.9, 29.4, 26.3, 22.8, 14.3.

<9-5> Synthesis of TPS-E7

According to the general synthetic procedures for de-O-benzoylation described in Example 3-3, TPS-E7 was prepared in a yield of 92%. ¹H NMR (400 MHz, CD₃OD): δ 4.95 (d, J=8.0 Hz, 1H), 4.85 (d, J=8.0 Hz, 1H), 4.66 (d, J=8.0 Hz, 1H), 4.38 (t, J=8.0 Hz, 2H), 4.25 (d, J=10.4 Hz, 1H), 4.12-3.95 (m, 3H), 3.90-3.82 (m, 6H), 3.75-3.59 (m, 6H), 3.46-3.18 (m, 28H), 1.60-1.51 (m, 6H), 1.39-1.28 (m, 24H), 0.90 (t, J=8.0 Hz, 9H); ¹³C NMR (100 MHz, CD₃OD): δ 104.7, 104.0, 103.4, 102.9, 102.6, 80.3, 80.2, 78.4, 78.0, 77.9, 77.8, 77.6, 76.0, 75.4, 75.2, 75.1, 72.5, 72.1, 71.7, 71.5, 71.3, 70.8, 70.2, 69.2, 63.3, 62.9, 62.7, 62.5, 46.8, 33.1, 30.8, 30.4, 27.5, 23.8, 14.6; HRMS (EI): calcd. for C₅₆H₁₀₄O₂₉ [M+Na]⁺ 1263.6561, observed 1263.6556.

### PREPARATION EXAMPLE 10

### Synthesis of TPS-E8

<10-1> Synthesis of 2,2-bis ((octyloxy)methyl)propane-1,3-diol (Compound B3)

According to the general synthetic procedures for a dialkylated diol described in Example 3-1, 2,2-bis ((octyloxy)methyl)propane-1,3-diol was prepared in a yield of 94%. ¹H NMR (400 MHz, CDCl₃): δ 3.65 (d, J=4.0 Hz, 4H), 3.51 (s, 4H), 3.42 (t, J=8.0 Hz, 4H), 2.85 (t, J=4.0 Hz, 2H), 1.56 (quin, J=4.0 Hz, 4H), 1.38-1.21 (m, 20H), 0.87 (t, J=8.0 Hz, 6H); ¹³C NMR (100 MHz, CDCl₃): δ 72.2, 71.8, 44.7, 31.9, 29.6, 29.5, 29.3, 26.3, 22.8, 14.2.

<10-2> Synthesis of 3-(octyloxy)-2,2-bis ((octyloxy)methyl)propan-1-ol (Compound C3)

According to the general synthetic procedures for a trialkylated mono-ol described in Example 3-2, 3-(octyloxy)-2,2-bis ((octyloxy)methyl)propan-1-ol was prepared in a yield of 85%. ¹H NMR (400 MHz, CDCl₃): δ 3.71 (d, J=8.0 Hz, 2H), 3.44 (s, 6H), 3.40 (t, J=8.0 Hz, 6H), 3.21 (t, J=8.0 Hz, 1H), 1.54 (quin, J=8.0 Hz, 6H), 1.40-1.21 (m, 30H), 0.87 (t, J=8.0 Hz, 9H); ¹³C NMR (100 MHz, CDCl₃): δ 71.6, 71.3, 66.3, 44.7, 31.8, 29.6, 29.5, 29.1, 26.2, 22.6, 14.1.

<10-3> Synthesis of Compound D3

According to the general synthetic procedures for glycosylation and de-O-benzoylation described in Example 3-3, compound D3 was prepared in a yield of 90%. ¹H NMR (400 MHz, CD₃OD): δ 4.21 (d, J=8.0 Hz, 1H), 3.85 (quint, J=8.0 Hz 2H), 3.70-3.65 (m, 1H), 3.51 (d, J=8.0 Hz, 1H), 3.45-3.36 (m, 12H), 3.32-3.28 (m, 2H), 3.26-3.15 (m, 2H), 1.59-1.50 (m, 6H), 1.40-1.22 (m, 30H), 0.90 (t, J=8.0 Hz, 9H); ¹³C NMR (100 MHz, CD₃OD): δ 105.5, 78.1, 77.9, 75.2, 72.6, 71.6, 70.6, 70.5, 62.8, 46.7, 33.2, 30.9, 30.7, 30.6, 27.5, 23.9, 14.7.

<10-4> Synthesis of TPS-E8a

According to the general synthetic procedures for glycosylation described in Example 3-3, TPS-E8a was prepared in a yield of 76%. ¹HNMR (400 MHz, CDCl₃): δ 8.24 (d, J=8.0 Hz, 2H), 8.15-7.78 (m, 24H), 7.72-7.67 (m, 5H), 7.66-7.59 (m, 3H), 7.58-7.12 (m, 46H), 5.96 (t, J=8.0 Hz, 1H), 5.92-5.82 (m, 3H), 5.71-5.69 (m, 2H), 5.61-5.42 (m, 6H), 5.01 (d, J=8.0 Hz, 1H), 4.98-4.92 (m, 2H), 4.88-4.79 (m, 2H), 4.77-4.65 (m, 2H), 4.59-4.41 (m, 3H), 4.36-4.29 (m, 1H), 4.26-4.13 (m, 2H), 4.12-4.05 (m, 2H), 4.03-3.95 (d, J=8.0 Hz, 1H), 3.91-3.71 (m, 4H), 3.37-3.18 (m, 14H), 2.85 (br s, 1H), 2.75 (br s, 1H), 1.59-1.45 (m, 6H), 1.40-1.19 (m, 30H), 0.84 (t, J=8.0 Hz, 9H); ¹³C NMR (100 MHz, CDCl₃): δ 166.2, 166.1, 166.0, 165.9, 165.8, 165.6, 165.2, 165.1, 164.9, 164.4, 133.4, 133.1, 130.1, 129.9, 129.8, 129.7, 129.5, 129.4, 129.3, 129.2, 129.1, 129.0, 128.9, 128.6, 128.5, 128.4, 128.3, 128.2, 101.7, 101.5, 99.9, 99.8, 74.9, 73.4, 72.8, 72.7, 72.6, 72.5, 72.4, 71.6, 71.3, 70.6, 70.3, 70.2, 69.4, 68.8, 63.7, 63.5, 45.6, 32.0, 29.9, 29.8, 26.4, 22.8, 14.3.

<10-5> Synthesis of TPS-E8

According to the general synthetic procedures for de-O-benzoylation described in Example 3-3, TPS-E8 was prepared in a yield of 92%. ¹H NMR (400 MHz, CD₃OD): δ 4.95 (d, J=8.0 Hz, 1H), 4.84 (d, J=8.0 Hz, 1H), 4.65 (d, J=8.0 Hz, 1H), 4.37 (t, J=8.0 Hz, 2H), 4.25 (d, J=10.4 Hz, 1H), 4.12-3.95 (m, 3H), 3.90-3.81 (m, 6H), 3.75-3.59 (m, 6H), 3.46-3.18 (m, 28H), 1.60-1.51 (m, 6H), 1.39-1.28 (m, 30H), 0.89 (t, J=8.0 Hz, 9H); ¹³C NMR (100 MHz, CD₃OD): δ 104.7, 104.0, 103.4, 102.9, 102.6, 80.3, 80.2, 78.4, 78.0, 77.9, 77.8, 77.6, 76.0, 75.4, 75.2, 75.1, 72.5, 72.1, 71.7, 71.5, 71.3, 70.8, 70.2, 69.2, 63.3, 62.9, 62.7, 62.5, 46.9, 33.2, 30.9, 30.7, 30.6, 27.6, 23.9, 14.6; HRMS (EI): calcd. for C₅₉H₁₁₀O₂₉ [M+Na]⁺ 1305.7030, observed 1305.7032.

### PREPARATION EXAMPLE 11

### Synthesis of TPS-E8L

<11-1> Synthesis of 2,2-bis ((octyloxy)methyl)propane-1,3-diol (Compound B3)

According to the general synthetic procedures for a dialkylated diol described in Example 3-1, 2,2-bis ((octyloxy)methyl)propane-1,3-diol was prepared in a yield of 94%. ¹H NMR (400 MHz, CDCl₃): δ 3.65 (d, J=4.0 Hz, 4H), 3.51 (s, 4H), 3.42 (t, J=8.0 Hz, 4H), 2.85 (t, J=4.0 Hz, 2H), 1.56 (quin, J=4.0 Hz, 4H), 1.38-1.21 (m, 20H), 0.87 (t, J=8.0 Hz, 6H); ¹³C NMR (100 MHz, CDCl₃): δ 72.2, 71.8, 44.7, 31.9, 29.6, 29.5, 29.3, 26.3, 22.8, 14.2.

<11-2> Synthesis of 3-(octyloxy)-2,2-bis ((octyloxy)methyl)propan-1-ol (Compound C3)

According to the general synthetic procedures for a trialkylated mono-ol described in Example 3-2, 3-(octyloxy)-2,2-bis ((octyloxy)methyl)propan-1-ol was prepared in a yield of 85%. ¹H NMR (400 MHz, CDCl₃): δ 3.71 (d, J=8.0 Hz, 2H), 3.44 (s, 6H), 3.40 (t, J=8.0 Hz, 6H), 3.21 (t, J=8.0 Hz, 1H), 1.54 (quin, J=8.0 Hz, 6H), 1.40-1.21 (m, 30H), 0.87 (t, J=8.0 Hz, 9H); ¹³C NMR (100 MHz, CDCl₃): δ 71.6, 71.3, 66.3, 44.7, 31.8, 29.6, 29.5, 29.1, 26.2, 22.6, 14.1.

<11-3> Synthesis of Compound D3

According to the general synthetic procedures for glycosylation and de-O-benzoylation described in Example 3-3, compound D3 was prepared in a yield of 90%. ¹H NMR (400 MHz, CD₃OD): δ 4.21 (d, J=8.0 Hz, 1H), 3.85 (quint, J=8.0 Hz 2H), 3.70-3.65 (m, 1H), 3.51 (d, J=8.0 Hz, 1H), 3.45-3.36 (m, 12H), 3.32-3.28 (m, 2H), 3.26-3.15 (m, 2H), 1.59-1.50 (m, 6H), 1.40-1.22 (m, 30H), 0.90 (t, J=8.0 Hz, 9H); ¹³C NMR (100 MHz, CD₃OD): δ 105.5, 78.1, 77.9, 75.2, 72.6, 71.6, 70.6, 70.5, 62.8, 46.7, 33.2, 30.9, 30.7, 30.6, 27.5, 23.9, 14.7.

<11-4> Synthesis of Compound E1

According to the general synthetic procedures for allylation and hydroboration described in Example 3-4, compound E1 was prepared in a yield of 80%. ¹H NMR (400 MHz, CD₃OD): δ 4.21 (d, J=8.0 Hz, 1H), 3.85-3.80 (m, 6H), 3.71-3.58 (m, 14H), 3.48 (d, J=8.0 Hz, 1H), 3.45-3.36 (m, 14H), 2.98 (t, J=8.0 Hz, 1H), 1.85-1.79 (m, 8H), 1.56-1.50 (m, 6H), 1.40-1.22 (m, 30H), 0.88 (t, J=8.0 Hz, 9H); ¹³C NMR (100 MHz, CD₃OD): δ 105.3, 86.1, 83.8, 79.4, 76.1, 72.6, 71.7, 71.1, 70.8, 70.7, 70.5, 70.0, 69.6, 60.4, 60.3, 60.2, 60.1, 46.6, 34.6, 34.4, 33.8, 33.2, 30.9, 30.8, 30.7, 27.6, 23.9, 14.8.

<11-5> Synthesis of TPS-E8La

According to the general synthetic procedures for glycosylation described in Example 3-3, TPS-E8La was prepared in a yield of 83%. ¹H NMR (400 MHz, CDCl₃): δ 8.15-7.78 (m, 30H), 7.58-7.12 (m, 50H), 6.12-5.95 (m, 4 H), 5.80-5.75 (m, 4H), 5.65-5.55 (m, 4H), 4.95-4.85 (m, 3H), 4.72-4.65 (m, 4H), 4.62-4.53 (m, 4H), 4.25-4.15 (m, 4H), 3.98-3.76 (m, 6H), 3.72-3.48 (m, 6H), 3.42-3.21 (m, 18H), 2.85 (br s, 2H), 2.65 (br s, 1H), 1.85-1.68 (m, 8H), 1.53-1.48 (m, 6H), 1.35-1.22 (m, 30H), 0.86 (t, J=8.0 Hz, 9H); ¹³C NMR (100 MHz, CDCl₃): δ 166.1, 165.8, 165.2, 165.1, 133.4, 133.2, 129.8, 129.7, 129.6, 129.4, 128.9, 128.5, 128.4, 128.3, 101.4, 73.1, 72.1, 72.0, 71.5, 69.8, 69.5, 69.1, 68.0, 67.5, 63.2, 59.9, 45.3, 31.9, 30.4, 29.7, 29.5, 29.4, 26.3, 22.7, 14.2.

<11-6> Synthesis of TPS-E8

According to the general synthetic procedures for de-O-benzoylation described in Example 3-3, TPS-E8L was prepared in a yield of 92%. ¹H NMR (400 MHz, CD₃OD): δ 4.23 (d, J=8.0 Hz, 4H), 4.16 (d, J=8.0 Hz, 1H), 4.02-3.99 (m, 4H), 3.92-3.80 (m, 10H), 3.73-3.55 (m, 17H), 3.42-3.15 (m, 32H), 1.92-1.80 (m, 8H), 1.55-1.50 (m, 6H), 1.41-1.25 (m, 30H), 0.89 (t, J=8.0 Hz, 9H); ¹³C NMR (100 MHz, CD₃OD): δ 104.5, 78.1, 77.9, 75.1, 72.6, 71.7, 70.5, 69.6, 68.1, 62.9, 46.6, 33.2, 31.9, 30.9, 30.8, 30.7, 30.6, 27.6, 23.9, 14.7. HRMS (EI): calcd. for C₇₁H₁₃₄O₃₃ [M+Na]⁺ 1537.8705, observed 1537.8701.

### PREPARATION EXAMPLE 12

### Synthesis of TPS-E9L

<12-1> Synthesis of 2,2-bis ((nonyloxy)methyl)propane-1,3-diol (Compound B4)

According to the general synthetic procedures for a dialkylated diol described in Example 3-1, 2,2-bis ((nonyloxy)methyl)propane-1,3-diol was prepared in a yield of 92%. ¹H NMR (400 MHz, CDCl₃): δ 3.65 (d, J=4.0 Hz, 4H), 3.51 (s, 4H), 3.42 (t, J=4.0 Hz, 4H), 2.85 (t, J=4.0 Hz, 2H), 1.56 (quin, J=4.0 Hz, 4H), 1.28-1.26 (m, 24H), 0.88 (t, J=7.2 Hz, 6H); ¹³C NMR (100 MHz, CDCl₃): δ 73.4, 72.2, 65.7, 44.6, 32.1, 29.8, 29.7, 29.5, 26.3, 22.9, 14.1.

<12-2> Synthesis of 3-(nonyloxy)-2,2-bis ((nonyloxy)methyl)propan-1-ol (Compound C4)

According to the general synthetic procedures for a trialkylated mono-ol described in Example 3-2, 3-(nonyloxy)-2,2-bis ((nonyloxy)methyl)propan-1-ol was prepared in a yield of 87%. ¹H NMR (400 MHz, CDCl₃): δ 3.71 (d, J=8.0 Hz, 2H), 3.43 (s, 6H), 3.38 (t, J=8.0 Hz, 6H), 3.17 (t, J=4.0 Hz, 1H), 1.53 (quin, J=4.0 Hz, 6H), 1.30-1.26 (m, 36H), 0.88 (t, J=7.2 Hz, 9H); ¹³C NMR (100 MHz, CDCl₃): δ 71.6, 71.3, 66.3, 44.7, 31.6, 29.5, 25.8, 22.6, 14.1.

<12-3> Synthesis of compound D4

According to the general synthetic procedures for glycosylation and de-O-benzoylation described in Example 3-3, compound D4 was prepared in a yield of 88%. ¹H NMR (400 MHz, CD₃OD): δ 4.21 (d, J=8.0 Hz, 1H), 3.85 (quint, J=8.0 Hz, 2H), 3.70-3.65 (m, 1H), 3.51 (d, J=8.0 Hz, 1H), 3.45-3.36 (m, 12H), 3.32-3.28 (m, 2H), 3.26-3.15 (m, 2H), 1.59-1.50 (m, 6H), 1.40-1.22 (m, 36H), 0.90 (t, J=8.0 Hz, 9H); ¹³C NMR (100 MHz, CD₃OD): δ 105.5, 78.1, 77.9, 75.2, 72.6, 71.6, 70.6, 70.5, 62.8, 46.7, 33.2, 31.0, 30.9, 30.8, 30.7, 27.6, 23.9, 14.7.

<12-4> Synthesis of Compound E2

According to the general synthetic procedures for allylation and hydroboration described in Example 3-4, compound E2 was prepared in a yield of 76%. ¹H NMR (400 MHz, CD₃OD): δ 4.21 (d, J=8.0 Hz, 1H), 3.85-3.80 (m, 6H), 3.71-3.58 (m, 14H), 3.48 (d, J=8.0 Hz, 1H), 3.45-3.36 (m, 14H), 2.98 (t, J=8.0 Hz, 1H), 1.85-1.79 (m, 8H), 1.56-1.50 (m, 6H), 1.40-1.22 (m, 36H), 0.88 (t, J=8.0 Hz, 9H); ¹³C NMR (100 MHz, CD₃OD): δ 105.3, 86.1, 83.7, 79.3, 76.1, 72.5, 71.6, 71.0, 70.8, 70.5, 70.0, 69.6, 60.4, 60.3, 60.2, 60.0, 46.6, 34.6, 34.4, 33.9, 32.8, 31.1, 31.0, 30.9, 30.8, 30.7, 30.2, 27.5, 23.9, 14.8.

<12-5> Synthesis of TPS-E9La

According to the general synthetic procedures for glycosylation described in Example 3-3, TPS-E9La was prepared in a yield of 84%. ¹H NMR (400 MHz, CDCl₃): δ 8.15-7.78 (m, 30H), 7.58-7.12 (m, 50H), 6.12-5.95 (m, 4 H), 5.80-5.75 (m, 4H), 5.65-5.55 (m, 4H), 4.95-4.85 (m, 3H), 4.72-4.65 (m, 4H), 4.62-4.53 (m, 4H), 4.25-4.15 (m, 4H), 3.98-3.76 (m, 6H), 3.72-3.48 (m, 6H), 3.42-3.21 (m, 18H), 2.85 (br s, 2H), 2.65 (br s, 1H), 1.85-1.68 (m, 8H), 1.53-1.48 (m, 6H), 1.35-1.22 (m, 36H), 0.86 (t, J=8.0 Hz, 9H); ¹³C NMR (100 MHz, CDCl₃): δ 166.1, 165.8, 165.2, 165.1, 133.4, 133.2, 129.8, 129.7, 129.6, 129.4, 129.3, 128.9, 128.5, 128.4, 128.3, 101.5, 73.1, 72.1, 72.0, 71.5, 69.8, 69.5, 69.1, 68.0, 67.5, 63.2, 63.1, 59.9, 45.3, 32.0, 31.9, 30.4, 29.8, 29.7, 29.5, 29.4, 26.3, 22.7, 14.2.

<12-6> Synthesis of TPS-E9L

According to the general synthetic procedures for de-O-benzoylation described in Example 3-3, TPS-E9L was prepared in a yield of 92%. ¹H NMR (400 MHz, CD₃OD): δ 4.23 (d, J=8.0 Hz, 4H), 4.16 (d, J=8.0 Hz, 1H), 4.02-3.99 (m, 4H), 3.92-3.80 (m, 10H), 3.73-3.55 (m, 17H), 3.42-3.15 (m, 32H), 1.92-1.80 (m, 8H), 1.55-1.50 (m, 6H), 1.41-1.25 (m, 36H), 0.89 (t, J=8.0 Hz, 9H); ¹³C NMR (100 MHz, CD₃OD): δ 104.5, 78.1, 77.9, 75.1, 72.6, 71.7, 70.5, 69.6, 68.1, 62.9, 46.6, 33.2, 31.9, 30.9, 30.8, 30.7, 30.6, 27.6, 23.9, 14.7; HRMS (0): calcd. for C₇₄H₁₄₀O₃₃ [M+Na]⁺ 1579.9175, observed 1579.9180.

### PREPARATION EXAMPLE 13

### Synthesis of TPS-E10L

<13-1> Synthesis of 2,2-bis ((decyloxy)methyl)propane-1,3-diol (Compound B5)

According to the general synthetic procedures for a dialkylated diol described in Example 3-1, 2,2-bis ((decyloxy)methyl)propane-1,3-diol was prepared in a yield of 92%. ¹H NMR (400 MHz, CDCl₃): δ 3.65 (d, J=4.0 Hz, 4H), 3.51 (s, 4H), 3.42 (t, J=4.0 Hz, 4H), 2.85 (t, J=4.0 Hz, 2H), 1.56 (quin, J=4.0 Hz, 4H), 1.28-1.26 (m, 28H), 0.88 (t, J=7.2 Hz, 6H); ¹³C NMR (100 MHz, CDCl₃): δ 73.4, 72.2, 65.7, 44.6, 32.1, 29.8, 29.7, 29.5, 26.2, 22.9, 14.1.

<13-2> Synthesis of 3-(decyloxy)-2,2-bis ((decyloxy)methyl)propan-1-ol (Compound C5)

According to the general synthetic procedures for a trialkylated mono-ol described in Example 3-2, 3-(decyloxy)-2,2-bis ((decyloxy)methyl)propan-1-ol was prepared in a yield of 90%. ¹H NMR (400 MHz, CDCl₃): δ 3.71 (d, J=8.0 Hz, 2H), 3.44 (s, 6H), 3.36 (t, J=8.0 Hz, 6H), 3.17 (t, J=8.0 Hz, 1H), 1.52 (quin, J=8.0 Hz, 6H), 1.28-1.26 (m, 42H), 0.88 (t, J=7.2 Hz, 9H); ¹³C NMR (100 MHz, CDCl₃): δ 71.6, 71.3, 66.3, 44.7, 31.8, 29.6, 29.1, 26.1, 22.6, 14.1.

<13-3> Synthesis of Compound D5

According to the general synthetic procedures for glycosylation and de-O-benzoylation described in Example 3-3, compound D5 was prepared in a yield of 88%. ¹H NMR (400 MHz, CD₃OD): δ 4.22 (d, J=8.0 Hz, 1H), 3.85 (quint, J=8.0 Hz, 2H), 3.70-3.65 (m, 1H), 3.51 (d, J=8.0 Hz, 1H), 3.45-3.36 (m, 12H), 3.32-3.28 (m, 2H), 3.26-3.15 (m, 2H), 1.59-1.50 (m, 6H), 1.40-1.22 (m, 42H), 0.88 (t, J=8.0 Hz, 9H); ¹³C NMR (100 MHz, CD₃OD): δ 105.5, 78.1, 77.9, 75.2, 72.6, 71.6, 70.6, 70.5, 62.8, 46.7, 33.2, 30.1, 30.9, 30.8, 30.6, 27.5, 23.9, 14.7.

<13-4> Synthesis of Compound E3

According to the general synthetic procedures for allylation and hydroboration described in Example 3-4, compound E3 was prepared in a yield of 76%. ¹H NMR (400 MHz, CD₃OD): δ 4.21 (d, J=8.0 Hz, 1H), 3.85-3.80 (m, 6H), 3.71-3.58 (m, 14H), 3.48 (d, J=8.0 Hz, 1H), 3.45-3.36 (m, 14H), 2.98 (t, J=8.0 Hz, 1H), 1.85-1.79 (m, 8H), 1.56-1.50 (m, 6H), 1.40-1.22 (m, 42H), 0.88 (t, J=8.0 Hz, 9H); ¹³C NMR (100 MHz, CD₃OD): δ 105.3, 86.0, 83.7, 79.3, 76.1, 72.5, 71.6, 71.0, 70.8, 70.5, 70.0, 69.6, 60.4, 60.3, 60.2, 60.0, 46.6, 34.6, 34.4, 33.8, 33.3, 31.1, 31.0, 30.9, 30.8, 30.7, 27.6, 23.9, 14.8.

<13-5> Synthesis of TPS-E10La

According to the general synthetic procedures for glycosylation described in Example 3-3, TPS-E10La was prepared in a yield of 84%. ¹H NMR (400 MHz, CDCl₃): δ 8.15-7.78 (m, 30H), 7.58-7.12 (m, 50H), 6.12-5.95 (m, 4 H), 5.80-5.75 (m, 4H), 5.65-5.55 (m, 4H), 4.95-4.85 (m, 3H), 4.72-4.65 (m, 4H), 4.62-4.53 (m, 4H), 4.25-4.15 (m, 4H), 3.98-3.76 (m, 6H), 3.72-3.48 (m, 6H), 3.42-3.21 (m, 18H), 2.85 (br s, 2H), 2.65 (br s, 1H), 1.85-1.68 (m, 8H), 1.53-1.48 (m, 6H), 1.35-1.22 (m, 42H), 0.86 (t, J=8.0 Hz, 9H); ¹³C NMR (100 MHz, CDCl₃): δ 166.1, 165.8, 165.2, 165.1, 133.4, 133.2, 129.8, 129.7, 129.6, 129.4, 129.3, 128.9, 128.5, 128.4, 128.3, 101.4, 73.1, 72.1, 72.0, 71.5, 69.8, 69.5, 69.1, 68.0, 67.5, 63.2, 63.1, 59.9, 45.3, 32.0, 31.9, 30.4, 29.7, 29.6, 29.5, 29.4, 26.3, 22.7, 14.2.

<13-6> Synthesis of TPS-E10L

According to the general synthetic procedures for de-O-benzoylation described in Example 3-3, TPS-E10L was prepared in a yield of 92%. ¹H NMR (400 MHz, CD₃OD): δ 4.23 (d, J=8.0 Hz, 4H), 4.16 (d, J=8.0 Hz, 1H), 4.02-3.99 (m, 4H), 3.92-3.80 (m, 10H), 3.73-3.55 (m, 17H), 3.42-3.15 (m, 32H), 1.92-1.80 (m, 8H), 1.55-1.50 (m, 6H), 1.41-1.25 (m, 42H), 0.88 (t, J=8.0 Hz, 9H); ¹³C NMR (100 MHz, CD₃OD): δ 104.5, 78.1, 77.9, 75.1, 72.6, 71.7, 70.5, 69.6, 68.1, 62.9, 46.6, 33.2, 31.9, 30.9, 30.8, 30.7, 30.6, 27.6, 23.9, 14.7; HRMS (EI): calcd. for C₇₇H₁₄₆O₃₃ [M+Na]⁺ 1621.9644, observed 1621.9640.

### PREPARATION EXAMPLE 14

### Synthesis of TPS-E11L

<14-1> Synthesis of 2,2-bis ((undecyloxy)methyl)propane-1,3-diol (Compound B6)

According to the general synthetic procedures for a dialkylated diol described in Example 3-1, 2,2-bis ((undecyloxy)methyl)propane-1,3-diol was prepared in a yield of 94%. ¹H NMR (400 MHz, CDCl₃): δ 3.65 (d, J=4.0 Hz, 4H), 3.51 (s, 4H), 3.42 (t, J=8.0 Hz, 4H), 2.85 (t, J=4.0 Hz, 2H), 1.56 (quin, J=4.0 Hz, 4H), 1.28-1.25 (m, 32H), 0.88 (t, J=7.2 Hz, 6H); ¹³C NMR (100 MHz, CDCl₃): δ 73.5, 72.3, 65.7, 44.6, 32.1, 29.8, 29.7, 29.6, 29.5, 26.3, 22.8, 22.9, 14.1.

<14-2> Synthesis of 3-(undecyloxy)-2,2-bis ((undecyloxy)methyl)propan-1-ol (Compound C6)

According to the general synthetic procedures for a trialkylated mono-ol described in Example 3-2, 3-(undecyloxy)-2,2-bis ((undecyloxy)methyl)propan-1-ol was prepared in a yield of 85%. ¹H NMR (400 MHz, CDCl₃): δ 3.71 (d, J=8.0 Hz, 2H), 3.43 (s, 6H), 3.38 (t, J=8.0 Hz, 6H), 3.16 (t, J=8.0 Hz, 1H), 1.53 (quin, J=8.0 Hz, 6H), 1.28-1.26 (m, 48H), 0.88 (t, J=7.2 Hz, 9H); ¹³C NMR (100 MHz, CDCl₃): δ 71.7, 71.4, 62.9, 45.1, 32.1, 29.9, 29.7, 29.5, 26.4, 22.7, 14.2.

<14-3> Synthesis of Compound D6

According to the general synthetic procedures for glycosylation and de-O-benzoylation described in Example 3-3, compound D6 was prepared in a yield of 88%. ¹H NMR (400 MHz, CD₃OD): δ 4.22 (d, J=8.0 Hz, 1H), 3.85 (quint, J=8.0 Hz, 2H), 3.70-3.65 (m, 1H), 3.51 (d, J=8.0 Hz, 1H), 3.45-3.36 (m, 12H), 3.32-3.28 (m, 2H), 3.26-3.15 (m, 2H), 1.59-1.50 (m, 6H), 1.40-1.22 (m, 48H), 0.88 (t, J=8.0 Hz, 9H); ¹³C NMR (100 MHz, CD₃OD): δ 105.5, 78.1, 77.9, 75.2, 72.6, 71.6, 70.6, 70.5, 62.8, 46.7, 33.2, 30.1, 30.9, 30.8, 30.7, 30.6, 27.5, 23.9, 14.7.

<14-4> Synthesis of Compound E4

According to the general synthetic procedures for allylation and hydroboration described in Example 3-4, compound E4 was prepared in a yield of 75%. ¹H NMR (400 MHz, CD₃OD): δ 4.21 (d, J=8.0 Hz, 1H), 3.85-3.80 (m, 6H), 3.71-3.58 (m, 14H), 3.48 (d, J=8.0 Hz, 1H), 3.45-3.36 (m, 14H), 2.98 (t, J=8.0 Hz, 1H), 1.85-1.79 (m, 8H), 1.56-1.50 (m, 6H), 1.40-1.22 (m, 48H), 0.89 (t, J=8.0 Hz, 9H); ¹³C NMR (100 MHz, CD₃OD): δ 105.3, 86.1, 83.7, 79.3, 76.1, 72.5, 71.6, 71.0, 70.8, 70.5, 70.0, 69.6, 60.4, 60.3, 60.2, 60.0, 46.6, 34.6, 34.4, 33.8, 33.3, 31.1, 31.0, 30.9, 30.8, 30.7, 27.6, 23.9, 14.8.

<14-5> Synthesis of TPS-E11La

According to the general synthetic procedures for glycosylation described in Example 3-3, TPS-E11La was prepared in a yield of 82%. ¹H NMR (400 MHz, CDCl₃): δ 8.15-7.78 (m, 30H), 7.58-7.12 (m, 50H), 6.12-5.95 (m, 4 H), 5.80-5.75 (m, 4H), 5.65-5.55 (m, 4H), 4.95-4.85 (m, 3H), 4.72-4.65 (m, 4H), 4.62-4.53 (m, 4H), 4.25-4.15 (m, 4H), 3.98-3.76 (m, 6H), 3.72-3.48 (m, 6H), 3.42-3.21 (m, 18H), 2.85 (br s, 2H), 2.65 (br s, 1H), 1.85-1.68 (m, 8H), 1.53-1.48 (m, 6H), 1.35-1.22 (m, 48H), 0.86 (t, J=8.0 Hz, 9H); ¹³C NMR (100 MHz, CDCl₃): δ 166.1, 165.8, 165.2, 165.1, 133.4, 133.2, 129.8, 129.7, 129.6, 129.4, 129.3, 128.9, 128.5, 128.4, 128.3, 101.4, 73.1, 72.1, 72.0, 71.5, 69.9, 69.8, 69.5, 69.1, 68.0, 67.5, 63.2, 63.1, 59.9, 45.3, 32.2, 32.0, 31.9, 30.4, 29.8, 28.7, 29.5, 29.4, 26.3, 22.8, 14.2.

<14-6> Synthesis of TPS-E11L

According to the general synthetic procedures for de-O-benzoylation described in Example 3-3, TPS-E11L was prepared in a yield of 92%. ¹H NMR (400 MHz, CD₃OD): δ 4.23 (d, J=8.0 Hz, 4H), 4.16 (d, J=8.0 Hz, 1H), 4.02-3.99 (m, 4H), 3.92-3.80 (m, 10H), 3.73-3.55 (m, 17H), 3.42-3.15 (m, 32H), 1.92-1.80 (m, 8H), 1.55-1.50 (m, 6H), 1.41-1.25 (m, 48H), 0.88 (t, J=8.0 Hz, 9H); ¹³C NMR (100 MHz, CD₃OD): δ 104.5, 78.1, 77.9, 75.1, 72.6, 71.7, 70.5, 69.6, 68.1, 62.9, 46.6, 33.2, 31.9, 30.9, 30.8, 30.7, 30.6, 27.6, 23.9, 14.7; FIRMS (EI): calcd. for C₈₀H₁₅₂O₃₃ [M+Na]⁺ 1664.0114, observed 1664.0107.

### EXAMPLE 4

### Method of Synthesizing TPS-As

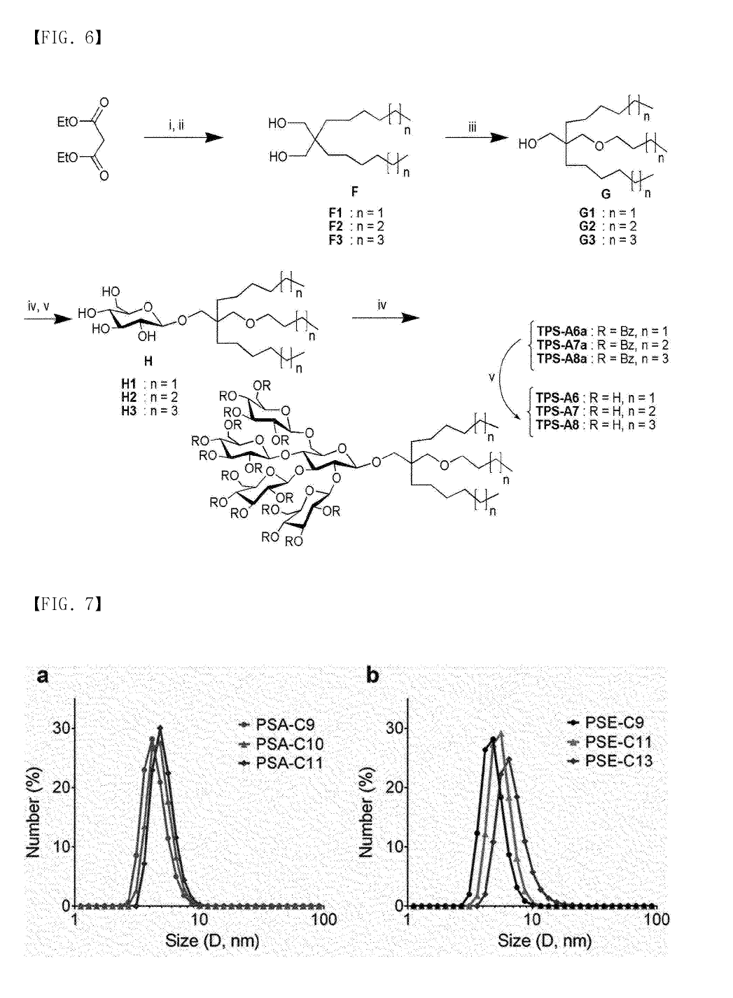

A synthetic scheme for TPS-As is shown in FIG. 6. Three kinds of tripod penta-saccharide amphiphiles (TPSs-As) were synthesized according to synthesis methods described in the following <4-1> to <4-5>.

<4-1> General Synthetic Procedures for Dialkylation and Reduction (Synthesis of Compound F, Steps i and ii in FIG. 6)

The method used to carry out this reaction is a modification of the method described in the journal article (P.S. Chae et al., Chem. Eur. J. 2013, 19, 15645-15651) published by the present inventors. A diethyl malonate (6.9 mmol) solution dissolved in THF was treated with NaH (21mmol) dissolved in THF at 0° C. and stirred for 20 minutes. Then, 1-iodoalkane (RI) (2.6 equivalent) was added to the reaction mixture. After addition, the reaction mixture was stirred at room temperature for 48 hours, and the reaction was terminated by adding ice-cold saturated NH₄Cl and extracted twice with diethyl ether. An organic layer was washed with brine and dried with anhydrous Na₂SO₄. After complete evaporation of the solvent, LiAlH₄ (14.0 mmol) was slowly added to residues dissolved in THF at 0° C. The mixture was stirred at room temperature for 4 hours, and the reaction was terminated by successive treatment with MeOH, water and a 1 N HCl aqueous solution at 0° C. and extracted twice with diethyl ether. A mixed organic layer was washed with brine and dried with anhydrous Na₂SO₄. The residues were purified using silica-gel column chromatography (EtOAc/hexane) to obtain an alkyl-containing diol (compound F) in the form of a white solid (yield of 90 to 92% (two steps)).

<4-2> General Synthetic Procedures for trialkylated mono-ol (Synthesis of Compound G, Step iii in FIG. 6)

In a two-neck flask filled with argon under anhydrous conditions, a solution, in which NaH (212.0 mmol) was stirred in dry DMF, was treated with a solution, in which a diol derivative (compound F) (212.0 mmol) was dissolved in dry DMF. After 20 minutes, the mixture was treated with a 1-bromoalkane (RBr) (330.0 mmol) and heated to 100° C. The reaction mixture was left at the same temperature for 4 hours, then cooled to room temperature, and the reaction was terminated with H₂O. The reaction mixture was extracted twice using CH₂Cl₂, washed with brine, and dried using anhydrous Na₂SO₄. The reaction mixture was purified using silica-gel column chromatography (EtOAc/hexane) to obtain a trialkyl-containing mono-ol (compound G) as an oily liquid (yield of 85 to 90%).

<4-3> General Procedures for Glycosylation Reaction and de-O-benzoylation Reaction Under Zemplen Conditions (Synthesis of Compound H, Steps iv and v in FIG. 6)

The method used to carry out this reaction is a modification of the method described in the journal article (P.S. Chae et al., Chem. Eur. J. 2013, 19, 15645-15651) published by the present inventors.

Briefly, a mixture of a mono-ol derivative (compound C) dissolved in anhydrous CH₂Cl₂ (30 mL), AgOTf (1.2 or 4.5 equiv.) and 2,4,6-collidine (0.7 or 2.0 equiv.) was stirred at −45° C. Next, perbenzoylated glucosylbromide (1.2 or 4.5 equiv.) dissolved in CH₂Cl₂ (30 mL) was transferred via a cannula to the solution over 30 minutes. The reaction product was allowed to warm to 0° C. for 1.5 hours. Progress of the reaction was monitored by TLC. After completion of the reaction (as determined by TLC), pyridine was added to the reaction mixture. The reaction mixture was diluted with CH₂Cl₂ and filtered through Celite. The filtrate was washed successively with a 1M Na₂S₂O₃ aqueous solution, 0.1M HCl aqueous solution and brine. Then, an organic layer was dried with anhydrous Na₂SO₄, and the solvent was removed using a rotary evaporator. Glycosylated residues were dissolved in MeOH and then a methanolic solution of 0.5 M NaOMe was added in a required amount so that the final concentration of NaOMe was 0.05 M. The reaction mixture was stirred at room temperature for 6 hours and then neutralized with Amberlite IR-120 (H⁺ form) resin. The resin was removed by filtration, washed with MeOH, and then the solvent was removed from the filtrate in vacuo. The residues were purified using silica-gel column chromatography (MeOH/CH₂Cl₂) to obtain a product (compound D) in the form of a white solid (yield of 88 to 90% (two steps)).

### PREPARATION EXAMPLE 15

### Synthesis of TPS-A6

<15-1> Synthesis of 2,2-dihexyl-propane-1,3-diol (Compound F1)

According to the general procedures for dialkylation and reduction described in Example 4-1, 2,2-dihexyl-propane-1,3-diol was prepared in a yield of 92%. ¹H NMR (400 MHz, CDCl₃): δ 3.57 (s, 4H), 2.28 (s, 2H), 1.38-1.08 (m, 20H), 0.88 (t, J=8.0 Hz, 6H); ¹³C NMR (100 MHz, CDCl₃): δ 69.5, 41.2, 32.0, 31.1, 30.5, 29.9, 23.1, 22.9, 14.3.

<15-2> Synthesis of 2-(butoxymethyl)-2-hexyloctan-1-ol (Compound G1)

According to the general synthetic procedures for a trialkylated mono-ol described in Example 4-2, 2-(butoxymethyl)-2-hexyloctan-1-ol was prepared in a yield of 90%. ¹H NMR (400 MHz, CDCl₃): δ 3.50 (d, J=8.0 Hz, 2H), 3.39 (t, J=8.0 Hz, 2H), 3.32 (s, 2H), 3.10 (t, J=4.0 Hz, 1H), 1.54 (quin, J=4.0 Hz, 2H), 1.40-1.19 (m, 22H), 0.88 (t, J=8.0 Hz, 9H); ¹³C NMR (100 MHz, CDCl₃): δ 79.0, 71.7, 70.2, 40.8, 32.0, 31.8, 31.5, 30.4, 23.0, 22.8, 19.5, 14.2, 14.0.

<15-3> Synthesis of Compound H1

According to the general synthetic procedures for glycosylation and de-O-benzoylation described in Example 4-3, compound H1 was prepared in a yield of 88%. ¹H NMR (400 MHz, CD₃OD): δ 4.19 (d, J=8.0 Hz, 1H), 3.87 (d, J=8.0 Hz, 1H), 3.84 (d, J=8.0 Hz, 1H), 3.75-3.67 (m, 1H), 3.39-3.20 (m, 10H), 1.56-1.50 (m, 2H), 1.40-1.22 (m, 22H), 0.89 (t, J=8.0 Hz, 9H); ¹³C NMR (100 MHz, CD₃OD): δ 105.4, 78.2, 77.9, 75.3, 74.3, 73.8, 72.1, 71.8, 62.9, 42.2, 33.1, 32.5, 31.4, 23.9, 23.8, 20.7, 14.6, 14.4.

<15-4> Synthesis of TPS-A6a

According to the general synthetic procedures for glycosylation described in Example 4-3, TPS-A6a was prepared in a yield of 80%. ¹H NMR (400 MHz, CDCl₃): δ 8.24 (d, J=8.0 Hz, 2H), 8.15-7.80 (m, 24H), 7.72-6.67 (m, 5H), 7.66-7.59 (m, 3H), 7.58-7.12 (m, 46H), 5.94 (t, J=8.0 Hz, 1H), 5.91-5.82 (m, 3H), 5.71-5.69 (m, 2H), 5.61-5.42 (m, 6H), 5.01 (d, J=8.0 Hz, 1H), 4.98-4.92 (m, 2H), 4.88-4.79 (m, 2H), 4.77-4.65 (m, 2H), 4.58-4.42 (m, 1H), 4.41-4.39 (m, 2H), 4.38-4.36 (m, 1H), 4.29-4.06 (m, 5H), 3.72-3.39 (m, 4H), 3.65 (t, J=8.0 Hz, 1H), 3.58 (d, J=8.0 Hz, 1H), 3.41-3.35 (m, 2H), 3.29-3.26 (br s, 1H), 3.18 (d, J=8.0 Hz, 1H), 3.08 (t, J=8.0 Hz, 2H), 2.75 (br s, 1H), 2.63 (br s, 1H), 1.56-1.50 (m, 2H), 1.40-1.15 (m, 22H), 0.84 (t, J=8.0 Hz, 9H); ¹³C NMR (100 MHz, CDCl₃): δ 166.2, 166.1, 166.0, 165.9, 165.6, 165.3, 165.2, 165.1, 164.9, 164.5, 164.4, 133.4, 133.2, 133.1, 130.1, 130.0, 129.9, 129.8, 129.7, 129.5, 129.4, 129.3, 129.2, 129.1, 129.0, 128.6, 128.5, 128.4, 128.3, 128.2, 101.6, 99.9, 73.4, 72.8, 72.6, 72.3, 71.8, 71.4, 71.2, 70.4, 63.7, 41.0, 32.2, 32.1, 32.0, 30.5, 30.4, 26.1, 23.0, 22.9, 22.6, 19.6, 14.3, 14.2.

<15-5> Synthesis of TPS-A6

According to the general synthetic procedures for de-O-benzoylation described in Example 4-3, TPS-A6 was prepared in a yield of 94%. ¹H NMR (400 MHz, CD₃OD): δ 4.93 (d, J=8.0 Hz, 1H), 4.81 (d, J=8.0 Hz, 1H), 4.62 (d, J=8.0 Hz, 1H), 4.33 (d, J=8.0 Hz, 2H), 4.24 (d, J=10.0 Hz, 1H), 4.02 (t, J=8.0 Hz, 1H), 3.91-3.85 (m, 2H), 3.87-3.82 (m, 5H), 3.68-3.63 (m, 6H), 3.48-3.19 (m, 22H), 1.52-1.46 (m, 2H), 1.38-1.18 (m, 22H), 0.86 (t, J=8.0 Hz, 9H); ¹³C NMR (100 MHz, CD₃OD): δ 104.8, 103.8, 103.3, 103.0, 102.5, 78.4, 78.1, 78.0, 77.8, 77.7, 76.1, 76.0, 75.3, 75.1, 75.0, 74.2, 74.0, 72.2, 71.7, 71.5, 71.4, 69.4, 63.4, 62.9, 62.8, 62.5, 42.3, 33.2, 33.1, 32.3, 32.2, 31.4, 23.8, 20.7, 14.6, 14.5; HRMS (EI): calcd. for C₄₉H₉₀O₂₇ [M+Na]⁺ 1133.5567, observed 1133.5564.

### PREPARATION EXAMPLE 16

### Synthesis of TPS-A7

<16-1> Synthesis of 2,2-diheptyl-propane-1,3-diol (Compound F2)

According to the general procedures for dialkylation and reduction described in Example 4-1, 2,2-diheptyl-propane-1,3-diol was prepared in a yield of 92%. ¹H NMR (400 MHz, CDCl₃): δ 3.57 (s, 4H), 2.28 (s, 2H), 1.38-1.08 (m, 24H), 0.88 (t, J=8.0 Hz, 6H); ¹³C NMR (100 MHz, CDCl₃): δ 69.5, 41.2, 32.1, 30.8, 29.8, 29.5, 23.1, 22.9, 14.3.

<16-2> Synthesis of 2-heptyl-2-((pentyloxy)methyl)nonan-1-ol (Compound G2)

According to the general synthetic procedures for a trialkylated mono-ol described in Example 4-2, 2-heptyl-2-((pentyloxy)methyl)nonan-1-ol was prepared in a yield of 90%. ¹H NMR (400 MHz, CDCl₃): δ 3.50 (d, J=8.0 Hz, 2H), 3.39 (t, J=8.0 Hz, 2H), 3.32 (s, 2H), 3.10 (t, J=4.0 Hz, 1H), 1.55 (quin, J=4.0 Hz, 2H), 1.40-1.19 (m, 28H), 0.88 (t, J=8.0 Hz, 9H); ¹³C NMR (100 MHz, CDCl₃): δ 79.1, 72.0, 70.3, 40.8, 32.1, 31.8, 31.5, 30.7, 29.7, 29.5, 26.0, 23.1, 22.8, 14.3, 14.1.

<16-3> Synthesis of Compound H2

According to the general synthetic procedures for glycosylation and de-O-benzoylation described in Example 4-3, compound H2 was prepared in a yield of 88%. ¹H NMR (400 MHz, CD₃OD): δ 4.20 (d, J=8.0 Hz, 1H), 3.86 (d, J=8.0 Hz 1H), 3.83 (d, J=8.0 Hz, 1H), 3.75-3.67 (m, 1H), 3.39-3.20 (m, 10H), 1.54-1.51 (m, 2H), 1.40-1.22 (m, 28H), 0.89 (t, J=8.0 Hz, 9H); ¹³C NMR (100 MHz, CD₃OD): δ 105.4, 78.2, 77.9, 75.2, 74.3, 73.8, 72.3, 71.7, 62.9, 42.2, 33.2, 32.4, 31.7, 30.6, 30.5, 29.9, 23.9, 23.8, 23.7, 14.8, 14.7.

<16-4> Synthesis of TPS-A7a

According to the general synthetic procedures for glycosylation described in Example 4-3, TPS-A7a was prepared in a yield of 82%. ¹H NMR (400 MHz, CDCl₃): δ 8.24 (d, J=8.0 Hz, 2H), 8.15-7.80 (m, 24H), 7.72-6.67 (m, 5H), 7.66-7.59 (m, 3H), 7.58-7.12 (m, 46H), 5.94 (t, J=8.0 Hz, 1H), 5.91-5.82 (m, 3H), 5.71-5.69 (m, 2H), 5.61-5.42 (m, 6H), 5.01 (d, J=8.0 Hz, 1H), 4.98-4.92 (m, 2H), 4.88-4.79 (m, 2H), 4.77-4.65 (m, 2H), 4.58-4.42 (m, 1H), 4.41-4.39 (m, 2H), 4.38-4.36 (m, 1H), 4.29-4.06 (m, 5H), 3.72-3.39 (m, 4H), 3.65 (t, J=8.0 Hz, 1H), 3.58 (d, J=8.0 Hz, 1H), 3.41-3.35 (m, 2H), 3.29-3.26 (br s, 1H), 3.18 (d, J=8.0 Hz, 1H), 3.08 (t, J=8.0 Hz, 2H), 2.75 (br s, 1H), 2.63 (br s, 1H), 1.56-1.50 (m, 2H), 1.40-1.15 (m, 28H), 0.84 (t, J=8.0 Hz, 9H); ¹³C NMR (100 MHz, CDCl₃): δ 166.2, 166.1, 166.0, 165.9, 165.6, 165.3, 165.2, 165.1, 164.9, 164.5, 164.4, 133.4, 133.2, 133.1, 130.1, 130.0, 129.9, 129.8, 129.7, 129.5, 129.4, 129.3, 129.2, 129.1, 129.0, 128.6, 128.5, 128.4, 128.3, 128.2, 101.6, 99.9, 73.4, 72.8, 72.6, 72.3, 71.8, 71.4, 71.2, 70.4, 63.7, 41.0, 32.1, 30.8, 30.7, 29.9, 29.7, 29.6, 26.1, 22.8, 22.7, 14.3, 14.2.

<16-5> Synthesis of TPS-A7

According to the general synthetic procedures for de-O-benzoylation described in Example 4-3, TPS-A7 was prepared in a yield of 94%. ¹H NMR (400 MHz, CD₃OD): δ 4.93 (d, J=8.0 Hz, 1H), 4.81 (d, J=8.0 Hz, 1H), 4.62 (d, J=8.0 Hz, 1H), 4.33 (d, J=8.0 Hz, 2H), 4.24 (d, J=10.0 Hz, 1H), 4.02 (t, J=8.0 Hz, 1H), 3.91-3.85 (m, 2H), 3.87-3.82 (m, 5H), 3.68-3.63 (m, 6H), 3.48-3.19 (m, 22H), 1.52-1.46 (m, 2H), 1.38-1.18 (m, 28H), 0.85 (t, J=8.0 Hz, 9H); ¹³C NMR (100 MHz, CD₃OD): δ 104.8, 103.8, 103.3, 103.0, 102.5, 78.4, 78.1, 78.0, 77.8, 77.7, 76.1, 76.0, 75.3, 75.1, 75.0, 74.2, 74.0, 72.2, 71.7, 71.5, 71.4, 69.4, 63.4, 62.9, 62.8, 62.5, 42.3, 33.2, 33.1, 32.2, 32.1, 31.7, 30.6, 30.5, 27.9, 24.1, 23.7, 14.6, 14.5; HRMS (EI): calcd. for C₅₂H₉₆O₂₇ [M+Na]⁺ 1175.6037, observed 1175.6033.

### PREPARATION EXAMPLE 17

### Synthesis of TPS-A8

<17-1> Synthesis of 2,2-dioctyl-propane-1,3-diol (Compound F3)

According to the general procedures for dialkylation and reduction described in Example 4-1, 2,2-dioctyl-propane-1,3-diol was prepared in a yield of 90%. ¹H NMR (400 MHz, CDCl₃): δ 3.57 (s, 4H), 2.28 (s, 2H), 1.38-1.08 (m, 28H), 0.88 (t, J=8.0 Hz, 6H); ¹³C NMR (100 MHz, CDCl₃): δ 69.5, 41.2, 32.1, 30.9, 30.8, 29.8, 29.5, 23.1, 22.9, 14.3.

<17-2> Synthesis of 2-((hexyloxy)methyl)-2-octyldecan-1-ol (Compound G3)

According to the general synthetic procedures for a trialkylated mono-ol described in Example 4-2, 2-((hexyloxy)methyl)-2-octyldecan-1-ol was prepared in a yield of 88%. ¹H NMR (400 MHz, CDCl₃): δ 3.50 (d, J=8.0 Hz, 2H), 3.39 (t, J=8.0 Hz, 2H), 3.32 (s, 2H), 3.10 (t, J=4.0 Hz, 1H), 1.54 (quin, J=4.0 Hz, 2H), 1.40-1.19 (m, 34H), 0.88 (t, J=8.0 Hz, 9H); ¹³C NMR (100 MHz, CDCl₃): δ 79.1, 72.0, 70.3, 40.8, 32.1, 31.8, 31.5, 30.7, 29.7, 29.5, 26.0, 23.1,22.9, 22.8, 14.3, 14.2.

<17-3> Synthesis of Compound H3

According to the general synthetic procedures for glycosylation and de-O-benzoylation described in Example 4-3, compound H3 was prepared in a yield of 86%. ¹H NMR (400 MHz, CD₃OD): δ 4.19 (d, J=8.0 Hz, 1H), 3.86 (d, J=8.0 Hz 1H), 3.84 (d, J=8.0 Hz, 1H), 3.75-3.67 (m, 1H), 3.39-3.20 (m, 10H), 1.54-1.50 (m, 2H), 1.40-1.22 (m, 34H), 0.89 (t, J=8.0 Hz, 9H); ¹³C NMR (100 MHz, CD₃OD): δ 105.4, 78.2, 77.9, 75.3, 74.3, 73.8, 72.4, 71.8, 62.9, 42.2, 33.2, 32.5, 31.8, 30.9, 30.8, 30.6, 27.3, 24.0, 23.9, 23.8, 23.7,14.8, 14.7.

<17-4> Synthesis of TPS-A8a

According to the general synthetic procedures for glycosylation described in Example 4-3, TPS-A8a was prepared in a yield of 78%. ¹H NMR (400 MHz, CDCl₃): δ 8.24 (d, J=8.0 Hz, 2H), 8.15-7.80 (m, 24H), 7.72-6.67 (m, 5H), 7.66-7.59 (m, 3H), 7.58-7.12 (m, 46H), 5.94 (t, J=8.0 Hz, 1H), 5.91-5.82 (m, 3H), 5.71-5.69 (m, 2H), 5.61-5.42 (m, 6H), 5.01 (d, J=8.0 Hz, 1H), 4.98-4.92 (m, 2H), 4.88-4.79 (m, 2H), 4.77-4.65 (m, 2H), 4.58-4.42 (m, 1H), 4.41-4.39 (m, 2H), 4.38-4.36 (m, 1H), 4.29-4.06 (m, 5H), 3.72-3.39 (m, 4H), 3.65 (t, J=8.0 Hz, 1H), 3.58 (d, J=8.0 Hz, 1H), 3.41-3.35 (m, 2H), 3.29-3.26 (br s, 1H), 3.18 (d, J=8.0 Hz, 1H), 3.08 (t, J=8.0 Hz, 2H), 2.75 (br s, 1H), 2.63 (br s, 1H), 1.56-1.50 (m, 2H), 1.40-1.15 (m, 34H), 0.84 (t, J=8.0 Hz, 9H); ¹³C NMR (100 MHz, CDCl₃): δ 166.2, 166.1, 166.0, 165.9, 165.6, 165.3, 165.2, 165.1, 164.9, 164.5, 164.4, 133.4, 133.2, 133.1, 130.1, 130.0, 129.9, 129.8, 129.7, 129.5, 129.4, 129.3, 129.2, 129.1, 129.0, 128.6, 128.5, 128.4, 128.3, 128.2, 101.6, 99.9, 73.4, 72.8, 72.6, 72.3, 71.8, 71.4, 71.2, 70.4, 63.7, 41.0, 32.2, 32.1, 31.9, 30.8, 30.7, 29.9, 29.7, 29.6, 26.1, 22.8, 22.6, 14.3, 14.2.

<17-5> Synthesis of TPS-A8

According to the general synthetic procedures for de-O-benzoylation described in Example 4-3, TPS-A8 was prepared in a yield of 93%. ¹H NMR (400 MHz, CD₃OD): δ 4.93 (d, J=8.0 Hz, 1H), 4.81 (d, J=8.0 Hz, 1H), 4.62 (d, J=8.0 Hz, 1H), 4.33 (d, J=8.0 Hz, 2H), 4.24 (d, J=10.0 Hz, 1H), 4.02 (t, J=8.0 Hz, 1H), 3.91-3.85 (m, 2H), 3.87-3.82 (m, 5H), 3.68-3.63 (m, 6H), 3.48-3.19 (m, 22H), 1.52-1.46 (m, 2H), 1.38-1.18 (m, 34H), 0.86 (t, J=8.0 Hz, 9H); ¹³C NMR (100 MHz, CD₃OD): δ 104.8, 103.8, 103.3, 103.0, 102.5, 78.4, 78.1, 78.0, 77.8, 77.7, 76.1, 76.0, 75.3, 75.1, 75.0, 74.2, 74.0, 72.2, 71.7, 71.5, 71.4, 69.4, 63.4, 62.9, 62.8, 62.5, 42.3, 33.2, 33.0, 32.3, 32.2, 31.7, 30.9, 30.8, 30.6, 30.5, 29.9, 23.8, 23.7, 14.6, 14.5; HRMS (EI): calcd. for C₅₅H₁₀₂O₂₇ [M+Na]⁺ 1217.6506, observed 1217.6509.

### EXAMPLE 5

### Characterization of PSAs and PSEs

To characterize PSAs of Preparation Examples 1 to 3 synthesized according to the synthetic method of Example 1; PSEs of Preparation Examples 4 to 7 synthesized according to the synthetic method of Example 2; TPS-Es and TPS-ELs of Preparation Examples 8 to 14 synthesized according to the synthetic method of Example 3; and TPS-As of Preparation Examples 15 to 17 synthesized according to the synthetic method of Example 4, the critical micellar concentrations (CMCs) of PSAs, PSEs and TPSs and the hydrodynamic radii (Rₕ) of formed micelles were measured.

Specifically, critical micellar concentrations (CMCs) were measured using hydrophobic fluorescent staining and diphenylhexatriene (DPH), and the hydrodynamic radii (Rₕ) of micelles formed by each compound at a concentration of 1.0 wt % were measured using dynamic light scattering (DLS). The results were compared with results obtained from an existing amphipathic molecule (detergent), DDM, and are shown in Table 1.

TABLE 1

| Detergent | M.W. | CMC (mM) | CMC (wt %) | Rₕ (nm) |
|---|---|---|---|---|
| PSA-C9 | 1109.3 | ~0.014 | ~0.0016 | 2.94 ± 0.05 |
| PSA-C10 | 1137.3 | ~0.009 | ~0.0010 | 3.10 ± 0.05 |
| PSA-C11 | 1165.4 | ~0.007 | ~0.0008 | 3.28 ± 0.03 |
| PSE-C7 | 1099.2 | ~0.27 | ~0.0300 | 2.66 ± 0.04 |
| PSE-C9 | 1155.3 | ~0.026 | ~0.0030 | 3.19 ± 0.04 |
| PSE-C11 | 1211.4 | ~0.004 | ~0.0005 | 3.46 ± 0.05 |
| PSE-C13 | 1267.5 | ~0.001 | ~0.0001 | 14.1 ± 0.24 |
| TPS-A6 | 1111.2 | ~0.07 | ~0.0078 | 2.4 ± 0.05 |
| TPS-A7 | 1153.3 | ~0.015 | ~0.0017 | 2.7 ± 0.05 |
| TPS-A8 | 1195.4 | ~0.007 | ~0.0008 | 9.1 ± 0.6 |
| TPS-E6 | 1199.3 | ~0.020 | ~0.0024 | 2.9 ± 0.05 |
| TPS-E7 | 1241.4 | ~0.012 | ~0.0015 | 13.0 ± 0.8 |
| TPS-E8 | 1283.5 | ~0.007 | ~0.0009 | 43.8 ± 16.3 |
| TPS-E8L | 1515.8 | ~0.006 | ~0.0009 | 4.5 ± 0.39 |
| TPS-E9L | 1557.9 | ~0.004 | ~0.0006 | 4.6 ± 0.18 |
| TPS-E10L | 1600.0 | ~0.002 | ~0.0003 | 4.8 ± 0.34 |
| TPS-E11L | 1642.1 | ~0.001 | ~0.0002 | 40.6 ± 4.4 |
| DDM | 510.1 | ~0.17 | ~0.0087 | 3.4 ± 0.03 |

The CMC values of PSAs, PSEs and TPSs were much smaller than the CMC value of DDM. Therefore, PSAs, PSEs and TPSs were capable of easily forming micelles with a small amount, and thus, self-assembly tendency was higher than DDM. In addition, the CMC values of PSAs, PSEs and TPSs decreased as the alkyl chain length thereof increased. This trend was consistent with the general idea that the hydrophobicity of amphipathic compounds is an important factor in determining CMC.

The size of micelles formed by PSAs, PSEs or TPSs exhibited a wide distribution, ranging from 2.4 to 60.1 nm. As the alkyl chain length of the compound increased, the size of the micelles tended to increase. PSE-C7 with the shortest alkyl chain formed the smallest micelles, whereas PSE-C13 with the longest alkyl chain formed the largest micelles. The micelle size of amphipathic compounds is known to be closely related to molecular geometry. Compounds with longer alkyl chains have a cylindrical shape and thus form larger micelles. The micelle size of PSA-C11, PSE-C9 and PSE-C11 was similar to the micelle size of DDM.

In addition, in the case of TPS-As and TPS-Es, it was determined that changes in the functional groups of the linker regions (alkyl to ether) were at least partly responsible for the difference in micelle size between the compounds. The size of micelles formed by TPS-ELs was relatively smaller than the size of micelles formed by TPS-Es. It was predicted that formation of relatively small micelles was possible because the hydrophilic group of TPS-ELs was geometrically much larger than the hydrophilic groups of TPS-As/-Es.

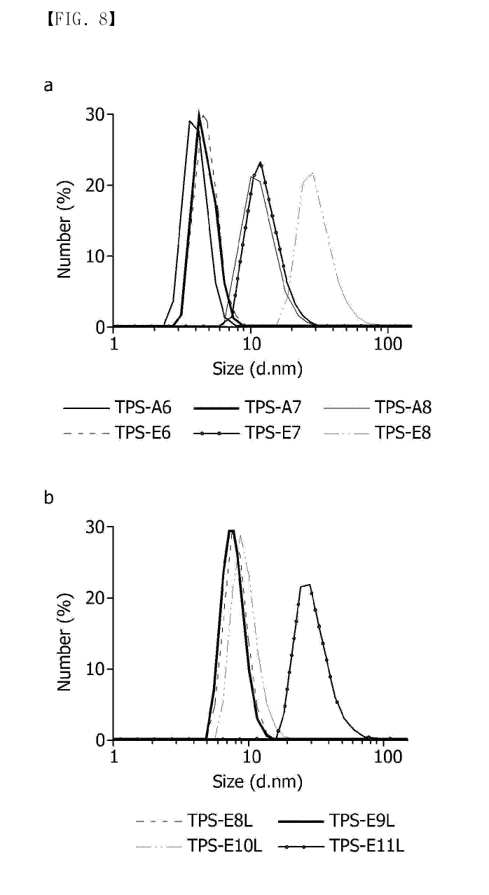

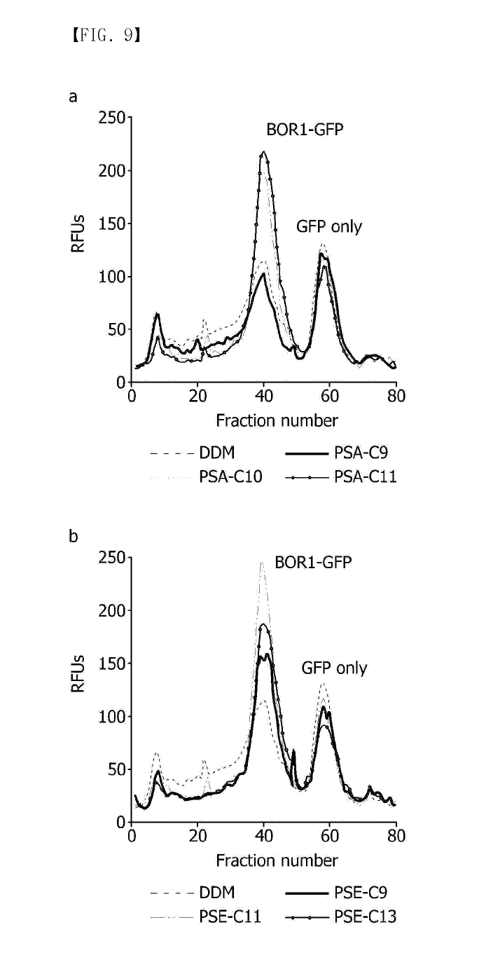

The measurement results of the size distribution of micelles formed by PSAs, PSEs, or TPSs using DLS are shown in FIGS. 7 and 8. When measuring, each of PSAs, PSEs and TPSs was used at a concentration of 1.0 wt %. As a result, the micelles of PSAs or PSEs had a single cluster and had high uniformity (FIG. 7). Every TPS-A, TPS-E and TPS-EL exhibited a single population in the average size distribution of micelles (FIG. 8).

From these results, it can be seen that since PSAs, PSEs or TPSs of the present invention have CMC values lower than the CMC value of DDM, micelles may be easily formed with a small amount, and self-assembly tendency is much larger than in DDM. The micelle size of PSA-C9, PSA-C10, PSA-C11, PSE-C9 and PSE-C11 is less than or equal to the micelle size of DDM and thus the compounds are expected to be useful for membrane protein studies similar to DDM.

### EXAMPLE 6

### Evaluation of PSAs and PSEs using Boron Transporter (BOR1) Membrane Protein

The ability of PSAs and PSEs to solubilize membrane proteins and to stabilize the structures of membrane proteins was evaluated using boron transporter (BOR1), a membrane protein. BOR1 was isolated from Arabidopsis thaliana, and was expressed in the form of a fusion protein having a GFP tag at the C-terminus in Saccharomyces cerevisiae FGY217 cells. This fusion protein has been proven to have boron transporting activity and is therefore involved in both structural and functional analysis of membrane proteins.

<6-1> Evaluation of Ability of PSAs and PSEs to Solubilize BOR1 Membrane Protein

The ability of amphipathic compound PSAs and PSEs to solubilize the boron transporter (BOR1) membrane protein was evaluated.

Specifically, membranes containing BOR1-GFP fusion proteins were treated with an existing amphipathic compound (DDM), PSAs (PSA-C9, PSA-C10 and PSA-C11), or PSEs (PSE-C9, PSE-C11 and PSE-C13) at a concentration of 1.0 wt %. The solubilization efficiencies of PSA-C9, PSA-C10 and PSE-C9 were about 80% similar to the solubilization efficiency of DDM. In particular, the BOR1-GFP protein was quantitatively extracted (˜100%) using PSE-C11 having medium length chains, thus confirming that PSE-C11 is particularly useful for solubilization of BOR1-GFP.

<6-2> Evaluation of Ability of PSAs and PSEs to Stabilize Structure of BOR1 Membrane Protein

The ability of PSAs or PSEs to stabilize the structure of boron transporter (BOR1) was measured in an aqueous solution. That is, the BOR1 protein solubilized in each amphipathic compound was denatured by heating, and then structural stability of BOR1 was measured using fluorescence-based size exclusion chromatography (FSEC).

Specifically, Arabidopsis thaliana-derived BOR1 was expressed in the form of a fusion protein having a GFP tag at the C-terminus in Saccharomyces cerevisiae FGY217 cells. The cells were grown in an URA-medium supplemented with 0.1% glucose. 2% galactose was added to the cell culture to induce protein expression and the cells were cultured at 20° C. for 18 hours (see Drew, D. et al., Nat. Protoc. 2008, 3, 784-798.). After culture, the cells were harvested and used to obtain the cell membranes (see Leung, J. et al., Protein Expr. Purif. 2010, 72, 139-146.). Membranes containing BOR1-GFP fusion proteins were diluted to a final total protein concentration of 2.8 mg/mL in PBS (pH7.4) supplemented with 1 wt % DDM, 1 wt % PSAs (PSA-C9, PSA-C10 or PSA-C11) or 1 wt % PSEs (PSE-C9, PSE-C11 or PSE-C13). The diluted samples were incubated at 4° C. for 1 hour with gentle shaking, and insoluble substances were removed by centrifugation at 14,000 g for 1 hour at 4° C. Supernatants containing the dissolved protein samples were incubated for 10 minutes at the specified temperature (35, 40, 45 or 50° C.), and strongly agglutinated proteins were removed by centrifugation at 14,000 g for 10 minutes at 4° C. 200 μL aliquots were taken from the supernatant and injected into a Superose 6 10/300 column equilibrated with 20 mM Tris-HCl (pH 7.5), 150 mM NaCl and 0.03% DDM. Each elution fraction was collected from a retention volume of 6.4 mL (i.e., after 6.4 mL of solution had passed), and added to a well of a clear bottom 96-well plate at a volume of 200 μL. GFP fluorescence generated in each fraction was measured using an excitation wavelength of 470 nm and an emission wavelength of 512 nm.

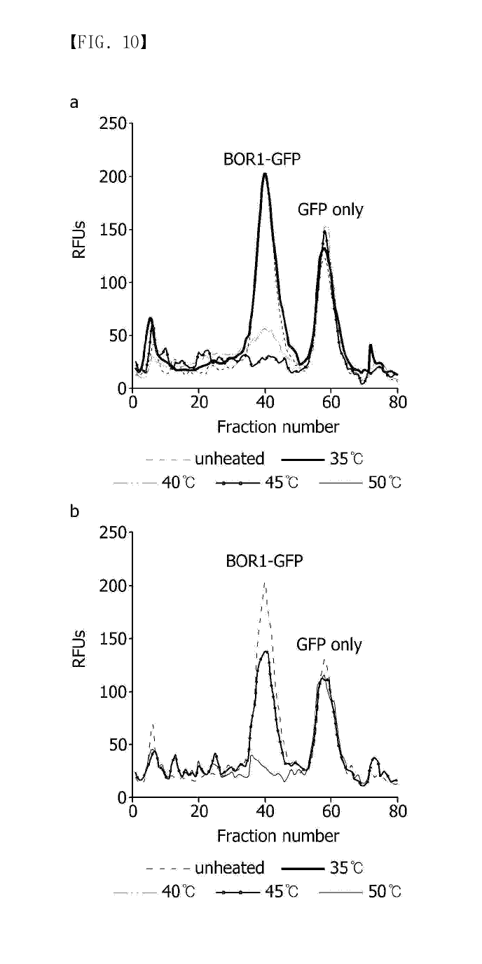

BOR1-GFP proteins solubilized in PSA (PSA-C9, PSA-C10, PSA-C11) (FIG. 9a) or PSE (PSE-C9, PSE-C11, PSE-C13) (FIG. 9b) were heated at 40° C. for 10 minutes and structural stability of the proteins was measured. The obtained results are shown in FIG. 9 compared with the results of DDM. The structural stability of proteins is represented in relative fluorescent units (RFUs). In the case of BOR1-GFP (fraction No. 40) solubilized in DDM, RFUs were relatively low due to denaturation after heating. When PSA-C10 and PSA-C11 were used as solubilizers, structural stability of BOR1-GFP was remarkably improved as compared to DDM and PSA-C9. These results indicate that BOR1 protein stability is dramatically improved in PSA-C10 and PSA-C11. PSA-C11 was slightly better than PSA-C10 for preventing protein denaturation and aggregation (FIG. 9a). When PSE formulations (PSE-C9, PSE-C11 and PSE-13) were used, structural stability of BOR1-GFP proteins after heating was greatly improved compared to DDM. The order of efficacy of PSE was PSE-C11>PSE-C13>PSE-C9 (FIG. 9b). In addition, PSE-C11 had a better stabilizing ability than PSA-C11, which was the best PSA, indicating that PSE-C11 having medium length alkyl chains is optimal for stabilizing BOR1 proteins.

FIG. 10 shows the results of measuring structural stability of BOR1-GFP proteins after heating BOR1-GFP proteins solubilized in DDM (FIG. 10a) or PSE-C11 (FIG. 10b) at 35, 40, 45 or 50° C. for 10 minutes. These results are representative of two independent experiments. BOR1 solubilized in DDM retained the original state thereof during incubation at 35° C. for 10 minutes. However, when temperature was increased to 40 or 45° C., BOR1 solubilized in DDM did not retain the protein structure and complete denaturation/aggregation occurred. On the other hand, BOR1 solubilized in PSE-C11 maintained monodispersibility even after heating to 45° C. These results indicate that the novel amphipathic compound, especially PSE-C11, is not only effective in solubilizing BOR1-GFP proteins, but also excellent for improving thermal stability of the fusion protein.

These results indicate that PSAs and PSEs are excellent in solubilizing BOR1, and are capable of stabilizing the BOR1 structure even at high temperatures. Therefore, it can be seen that PSAs and PSEs may be used to extract or stabilize membrane proteins.

### EXAMPLE 7

### Evaluation of Ability of PSAs, PSEs and TPSs to Stabilize Leucine Transporter (LeuT) Membrane Protein

The ability of PSAs, PSEs or TPSs (TPS-A/E/ELs) to stabilize leucine transporter (LeuT), a membrane protein, was measured. LeuT purified by DDM was mixed with a solution containing each amphipathic compound, and the mixture was incubated at room temperature for 12 days. The activity of LeuT proteins was measured by scintillation proximity assay (SPA) using a ligand ([³H]-Leu), and the concentration of PSAs, PSEs, TPSs or DDM was (a) CMC+0.04 wt %, or (b) CMC+0.2 wt %.

Specifically, LeuT stability measurement was performed in the following manner. According to the results of Example 6, PSA-C10, PSA-C11, PSE-C9, PSE-C11, and PSE-C13 were selected as the amphipathic compounds of PSAs and PSEs except for PSA-C9, and wild type leucine transporter (LeuT) was purified from Aquifex aeolicus using TPS-As, TPS-Es and TPS-EL, according to the method described in G. Deckert et al. (Nature 1998, 392, 353-358.). E. coli C41 (DE3) was transformed with a pET16b plasmid containing a gene construct encoding C-terminal 8×His-tagged LeuT, and thus LeuT was expressed in the form of a fusion protein having 8×His tags at the C-terminus in the transformed E. coli C41 (the expression plasmid was provided by Dr. E. Gouaux, Vollum Institute, Portland, Oreg., USA). Briefly, bacterial membranes containing LeuT were treated with 1.0 wt % DDM, and proteins were bound to Ni²⁺-NTA resin (Life Technologies, Denmark). LeuT bound to the resin was eluted using a buffer solution containing 20 mM Tris-HCl (pH 8.0), 1 mM NaCl, 199 mM KCl, 0.05% DDM and 300 mM imidazole. Next, about 1.5 mg/mL of protein stock was diluted 10-fold in an equivalent buffer supplemented with TPS-As, TPS-Es, TPS-ELs, PSAs/PSEs (PSA-C10, PSA-C11, PSE-C9, PSE-C11 and PSE-C13) or DDM (control group) to a final concentration of CMC+0.04 wt % or CMC+0.2 wt % without DDM and imidazole. The protein samples were stored at room temperature and centrifuged at designated times. Protein activity was determined by measuring the degree of binding to [³H]-Leu using scintillation proximity assay (SPA) (M. Quick et al., Proc. Natl, Acad. Sci. U.S.A. 2007, 104, 3603-3608.). Briefly, SPA was performed with 5 μL of each protein sample dissolved in a buffer containing 450 mM NaCl and each test compound. The SPA reaction was performed in the presence of 20 nM [³H]-Leu and copper chelate (His-Tag) YSi beads (both were purchased from PerkinElmer, Denmark). The degree of binding to [³H]-Leu was measured using a MicroBeta liquid scintillation counter (PerkinElmer).

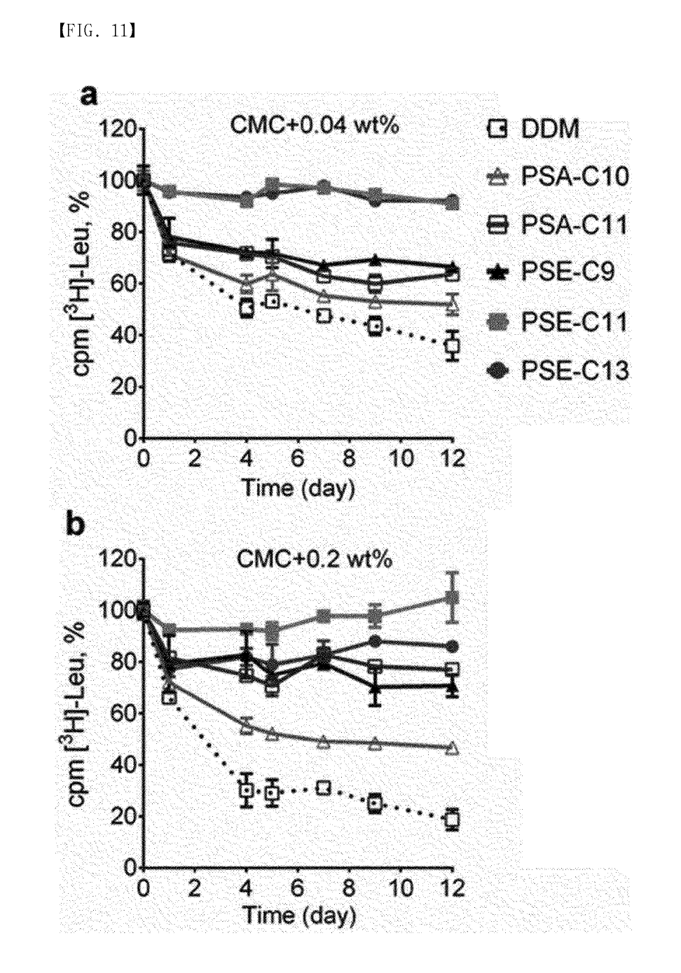

As shown in FIG. 11, both tested PSAs and PSEs showed better LeuT protein stabilizing ability than DDM at both concentrations. Specifically, in the case of the concentration of CMC+0.04 wt %, all tested PSAs were more effective than DDM in maintaining LeuT activity, and PSA-C11 was better than PSA-C10. PSE-C9 was the least effective among PSEs, but similar to PSA-C11, the best PSA. In particular, PSE-C11 and PSE-C13 were significantly superior to other tested compounds and DDM. LeuT solubilized by PSE-C11 and PSE-C13 did not show any appreciable decrease in protein activity after 12 days of incubation (FIG. 11a). In addition, a similar tendency was observed when the concentration of amphipathic molecules was increased to CMC+0.2 wt %. In particular, it is noteworthy that proteins solubilized in PSE-C11 retained 100% transporter activity after 12 days (FIG. 11b).

From the above results, it can be seen that, compared with DDM, all evaluated PSAs and PSEs are superior in maintaining LeuT activity, and PSEs have better overall performance than PSAs. In particular, PSE-C11 was optimal in maintaining transporter activity at both low and high concentrations, which is consistent with the results observed in the case of BOR1-GFP fusion proteins.

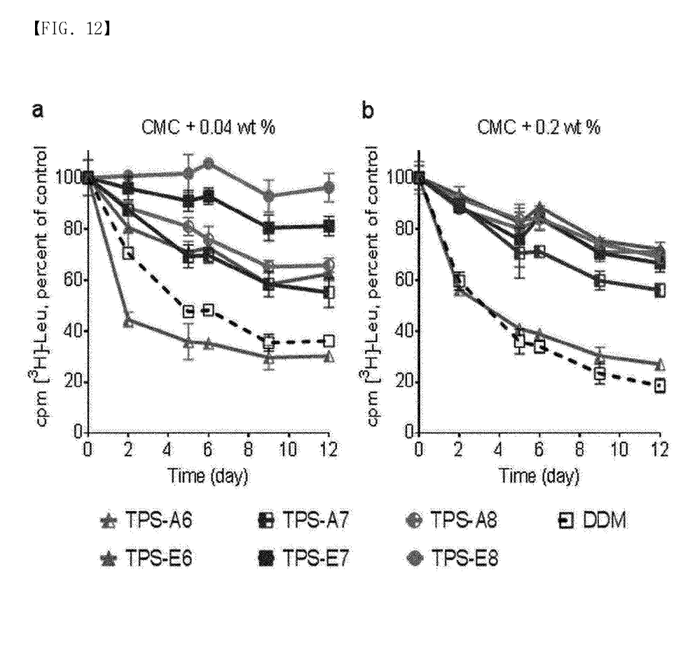

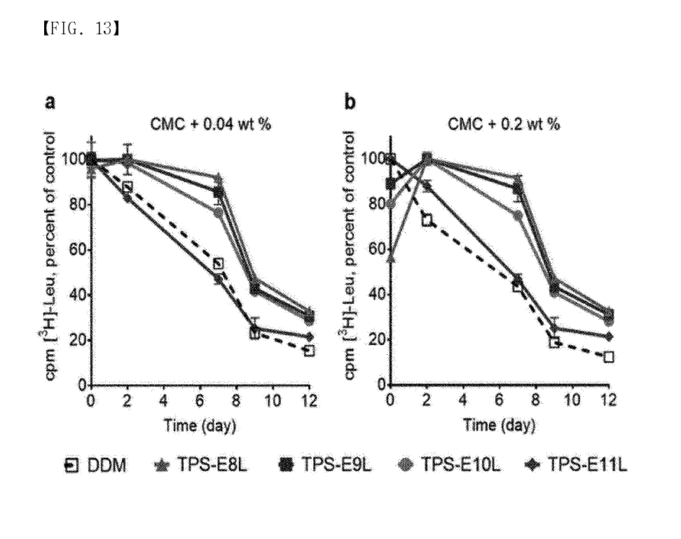

In addition, as shown in FIGS. 12 and 13, all tested amphipathic compounds except TPS-A6 were substantially better than DDM. The performance of TPS-E8 was highest among TPS-As and TPS-Es, and when the transporter was solubilized in TPS-E8, the transporter activity remained intact during 12 days of incubation (FIG. 12). The ability to maintain the stability of the transporter improved as the alkyl chain length of the amphipathic compound increased (FIG. 12). Also, a similar trend was observed when increasing the concentration of the amphipathic compounds to CMC+0.2 wt % (FIG. 12). In the case of TPS-ELs, the transporter activity was reduced overall as compared to the transporter activity observed in the case of TPS-As/Es (FIG. 13). The importance of the hydrophilic group of the amphipathic compound was demonstrated from the comparison of TPS-E8 and TPS-E8L. Both compounds had identical alkyl chains and linkers with similar hydrophilic groups, but TPS-E8 was superior to TPS-E8L. This suggests that TPS-E8 is more advantageous than TPS-E8L in maintaining LeuT stability because there is no propyl spacer in the hydrophilic group of TPS-E8. Overall, TPS-Es, especially TPS-E7 and TPS-E8, were superior to TPS-As and TPS-ELs in long term substrate binding capacity of transporters.

### EXAMPLE 8

### Evaluation of Ability of PSAs and PSEs to Solubilize and Stabilize Membrane Proteins using Salmonella typhimurium Melibiose Permease (MelB) Membrane Protein

The ability of PSAs or PSEs to extract (solubilize) MelBₛₜ (Salmonella typhimurium melibiose permease) proteins and stabilize the structures thereof was measured. MelB proteins were extracted using PSAs, PSEs or DDM, and the amount of the extracted proteins and structural stability thereof were quantitatively analyzed by SDS-PAGE and western immunoblotting. The protein extraction efficiency and thermally stabilizing ability of the amphipathic compounds were simultaneously evaluated by extracting MelB proteins at four temperatures (0, 45, 55 or 65° C.) for 90 minutes using the amphipathic compounds at a concentration of 1.5 wt %.

Specifically, according to the method described in the journal article (P. S. Chae, et al., Chemistry. 2013, 19, 15645-15651.) published by the present inventors, proteins (MelBₛₜ) were produced using a plasmid pK95AHB/WT MelBₛₜ/CH10 containing a gene construct encoding wild-type MelB having 10×His tags at the C-terminus and Salmonella typhimurium DW2 cells (melB and lacZY). According to the method described in the journal article (A. S. Ethayathulla et al., Nat. Commun. 2014, 5, 3009), cells were cultured and the cell membranes were obtained from the cultured cells. Protein analysis was performed using a Micro BCA kit (Thermo Scientific, Rockford, Ill.). To measure solubilization/stability, membrane samples (the final concentration of membrane proteins was 10 mg/mL) containing MelBₛₜ were mixed with a solubilization buffer solution (20 mM sodium phosphate, pH 7.5, 200 mM NaCl, 10% glycerol and 20 mM melibiose) containing DDM, PSAs (PSA-C11) or PSEs (PSE-C9, PSE-C11, PSE-C13) at a concentration of 1.5% (w/v). Extracts were incubated at four different temperatures (0, 45, 55 and 65° C.) for 90 minutes. Fractions were removed by ultracentrifugation at 355,590 g for 45 minutes using a Beckman Optima™ MAX ultracentrifuge equipped with a No. 4 TLA-100 rotor. 20 μg of proteins was separated using a 15% SDS-PAGE gel. Next, immunoblotting was performed using Penta-His-HRP antibodies (Qiagen, Germantown, Md.). After performing SDS-PAGE and immunoblotting, the amount of solubilized MelB was quantitated and expressed as a percentage of the amount of MelB measured in the control group.

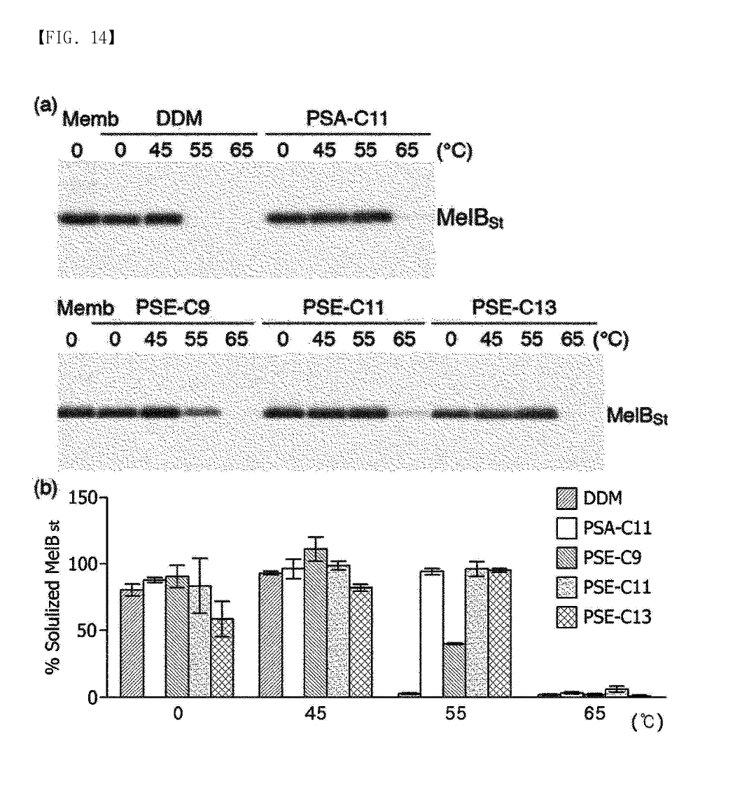

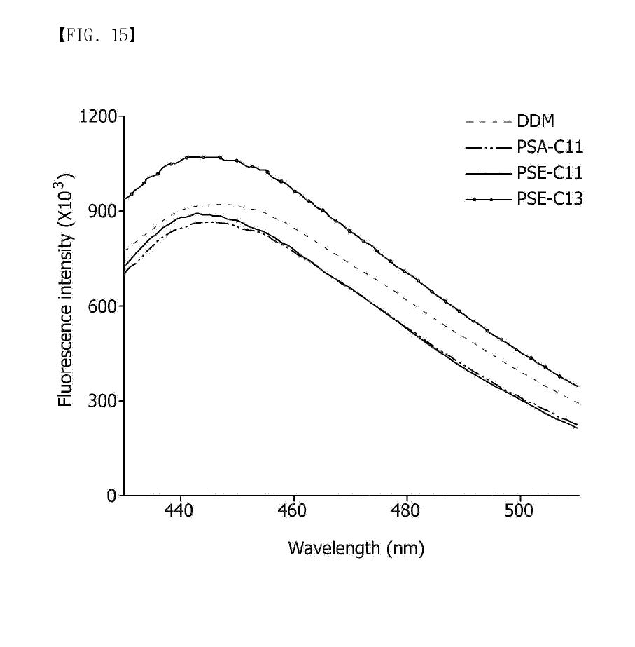

As shown in FIG. 14, when PSA-C11, PSE-C9, and PSE-C11 were used, MelB was extracted at an efficiency similar to the efficiency observed in the case of DDM at 0° C., and similar results were observed at 45° C. However, when the incubation temperature was increased to 55° C., a large difference was observed between DDM and PSAs/PSEs. At this temperature, all MelB dissolved in DDM disappeared in the solution, whereas all MelB surrounded by the novel amphipathic molecules (PSA-C11, PSE-C11 and PSE-C13) remained dissolved in the solution. These results indicate that MelB proteins in the novel amphipathic molecules have improved stability. When incubated at 65° C., a small amount of dissolved MelB was observed only in PSE-C11. Therefore, it was confirmed that PSAs and PSEs of the present invention are superior to DDM in stabilizing MelB proteins, and that PSAs and PSEs are excellent in extracting MelB proteins. In particular, PSE-C11 was the best among the novel amphipathic compounds tested with respect to MelB, which was consistent with the results obtained in the case of BOR1 and LeuT.

### EXAMPLE 9

### Evaluation of TPSs, PSAs and PSEs Using Human β₂ Adrenergic Receptor (β₂AR) Membrane Protein

The effects of TPSs, PSAs and PSEs on structural stability of human β₂ adrenergic receptor (β₂AR) and G protein-coupled receptor (GPCR) and the size of protein complexes were investigated. Based on the results obtained with BOR1, LeuT and MelB, TPSs (TPS-As, TPS-Es and TPS-ELs), PSA-C11, PSE-C11 and PSE-C13 were selected and used as test compounds.

<9-1> Evaluation of Structural Stability of Monobromobimane-Labeled β2ARs (mBBr-β2ARs) Solubilized in PSAs and PSEs Under Presence of High-Affinity Agonist BI (BI-167107)

The structural stability of monobromobimane-labeled β2ARs (mBBr-β2ARs) solubilized in PSAs and PSEs was evaluated in the presence of a high-affinity agonist BI (BI-167107).

Specifically, monobromobimane (mBBr)-labeled β2ARs (mainly labeled on Cys265) was used to measure changes in a fluorescence spectrum induced by a local structural change near transmembrane helix 6 (TM6) (see Yao, X. et al., Nat. Chem. Biol. 2006, 2, 417-422.). 0.5 μL of BI (agonist)-bound mBBr-β2ARs dissolved at a concentration of 0.5 μM in 0.1 wt % DDM was diluted with 500 μL of a buffer solution containing 0.1 wt % of each of the novel amphipathic compounds (PSA-C11, PSE-C11 and PSE-C13). After incubation of the protein samples for 30 minutes, the spectrum of mBBr was measured and compared with the spectrum of mBBr-labeled receptors dissolved in 0.1 wt % DDM. The 370 nm excitation wavelength was used for bimane fluorescence, and the emission spectra were measured at 430 to 510 nm using a Spex FluoroMax-3 spectrometer (Jobin Yvon Inc.) at 0.5 nm s⁻¹ in 1-nm units, and the photon counting mode was set to a 4-nm emission bandwidth pass. mBBr-labeled β2ARs dissolved in DDM were used as a positive control group. Data is representative of three independent experiments.

As shown in FIG. 15, the receptors dissolved in PSE-C13 showed a somewhat different bimane fluorescence spectrum compared to the receptors dissolved in DDM, whereas the spectra obtained from the receptors dissolved in PSA-C11 and PSE-C11 were very similar to that obtained from the receptors dissolved in DDM. These results indicated that the structural form of β2AR solubilized in PSA-C11 and PSE-C11 in the presence of BI (BI-167107) was very similar to the structural form of the receptor solubilized in DDM.

<9-2> Evaluation of Structural Change and Structural Stability of mBBr-β2ARs by PSAs/PSEs and DDM According Presence or Absence of Full Agonist (ISO) or Combination of ISO and G-Protein

The structural change and structural stability of mBBr-β2ARs by PSAs/PSEs and DDM according the presence or absence of a full agonist (isoproterenol, ISO) or the combination of ISO and a G-protein were measured. It is well known that a full agonist (e.g., BI) and the binding of a Gₛ-protein are simultaneously required for full activation of receptors (see Rasmussen, S. G. F. et al., Nature 2011, 469, 175-180.).

Specifically, G protein coupling experiments were performed using the following method. 0.5 μL of non-ligand mBBr-labeled receptors at a concentration of 50 μM was diluted with 500 μL of a buffer solution containing a 0.1 wt % amphipathic compound at room temperature for 15 minutes. After dilution, a dilution containing the receptors at a final concentration of 50 nM was obtained. 2 μM isoproterenol (ISO) was added to the diluted solution, and the solution was incubated for an additional 15 minutes. 250 nM Gₛ-protein was additionally added to the solution, and then the protein samples included in the solution were incubated at room temperature for an additional 20 minutes. The 370 nm excitation wavelength was used for bimane fluorescence, and the emission spectra were measured at 430 to 510 nm using a Spex FluoroMax-3 spectrometer (Jobin Yvon Inc.) at 0.5 nm s⁻¹ in 1-nm units, and the photon counting mode was set to a 4-nm emission bandwidth pass. The same experiment was repeated using 0.1 wt % DDM, which was used as a positive control. Data is representative of three independent experiments.

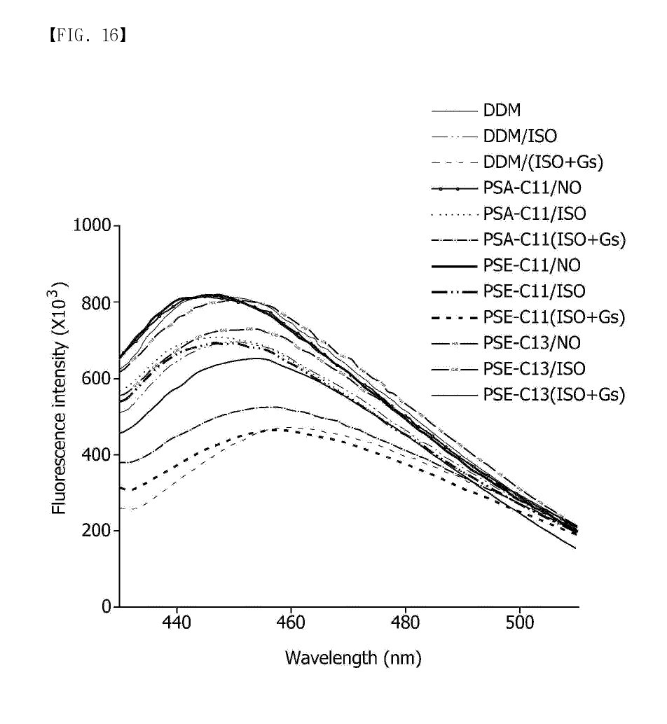

As shown in FIG. 16, when isoproterenol (ISO), a full agonist, was present, the receptors solubilized by PSA-C11 or PSE-C11 exhibited spectra similar to the receptor solubilized by DDM, indicating that the receptors were partially active in the presence of ISO. In addition, when a G-protein was added, additional changes were observed in the bimane fluorescence spectrum of β2AR, indicating that the receptor structure changed from partial to fully active. This structural change can be confirmed by a reduction in fluorescence intensity and a red-shift of the maximum emission wavelength.

These results indicate that PSA-C11 or PSE-C11 is functioning well for receptor activation by G-protein coupling, and suggest that the structures of β2ARs solubilized in PSA-C11 or PSE-C11 behaves in a similar manner to receptors present in the cell membranes.

<9-3> Measurement of Binding Activity of mBBr-β2ARs to Ligand (DHA) Using Radioactive Ligand Binding Assay

The membrane protein stabilizing ability of receptors (mBBr-β2ARs) solubilized in PSAs or PSEs was evaluated by measuring the binding activity of the receptors to [³H]-dihydroalprenolol ([³H]-DHA).

Specifically, radioactive ligand binding assay was performed as follows. β2ARs purified with 0.1 wt % DDM was concentrated to about 10 mg/mL (approximately 200 μM). β2ARs purified with DDM were used to prepare a master binding mixture containing 10 nM [³H]-dihydroalprenolol (DHA), which was dissolved in 0.2 wt % of DDM, PSA or PSE and supplemented with 0.5 mg/mL BSA. At a concentration of 0.2 pmol, the activity of the receptors purified with the amphipathic compounds was monitored at regular intervals over a 4-day incubation period. Protein samples were incubated on ice for 2 days and then at room temperature for 2 days. Receptor activity was measured by binding assay for dissolved radioactive ligands. The receptors purified with DDM or a novel formulation (PSA-C11, PSE-C11 or PSE-C13) were incubated with 10 nM [³H]-DHA at room temperature for 30 minutes. The mixture was loaded onto a G-50 column, a solution passed through the column was collected in 1 mL of a binding buffer (100 mM NaCl and 20 mM HEPES (pH 7.5) supplemented with 0.5 mg/mL BSA and 20×CMC amphipathic compounds), and 15 mL of a scintillation fluid was added to the solution. Receptor-bound [³H]-DHA was measured using a scintillation counter (Beckman). Non-specific binding of [³H]-DHA was measured by adding 1 μM alprenolol (Sigma) to the same reaction mixture. The binding degree of the receptors to [³H]-DHA was represented by a column graph.

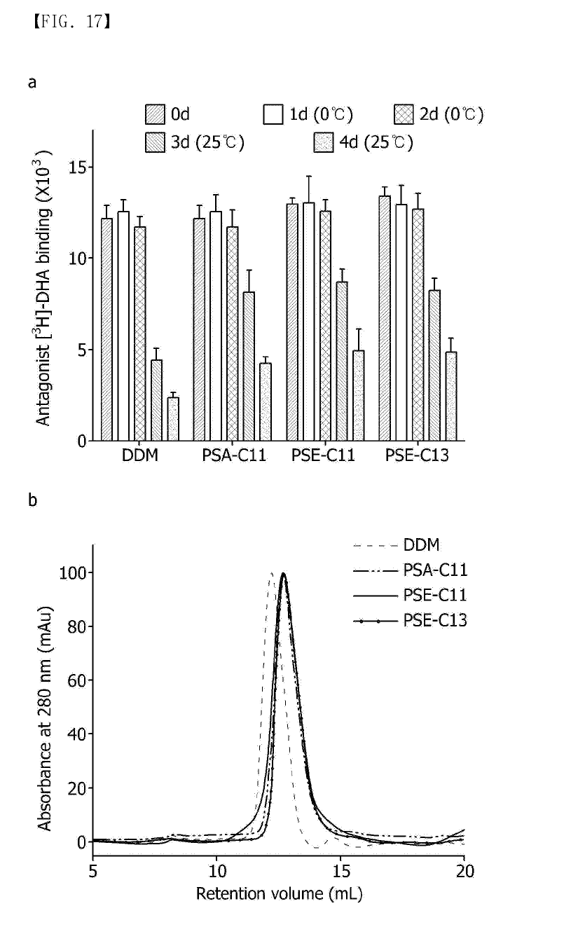

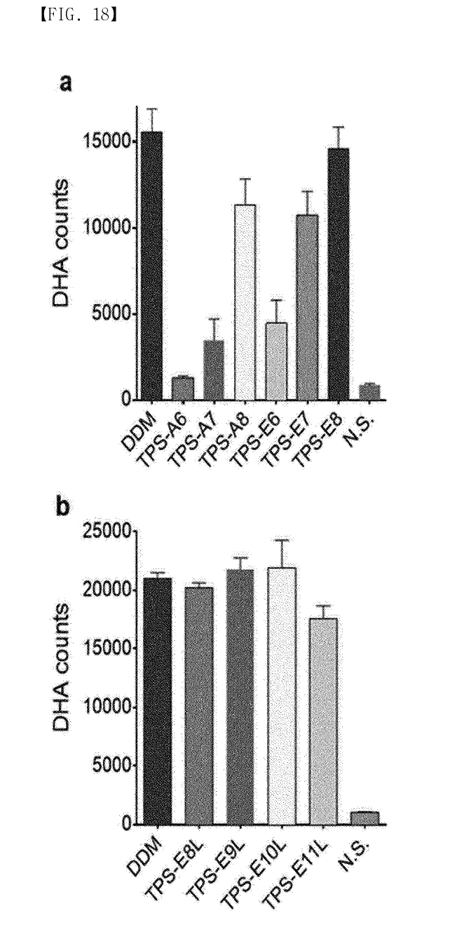

As shown in FIG. 17a, all receptors purified with DDM, PSA-C11, PSE-C11 or PSE-C13 well maintained the initial activity thereof during the first two days of incubation at 0° C. It should be noted that the receptors solubilized in PSE-C11 or PSE-C13 had higher initial activity than proteins solubilized in DDM. When incubation temperature was increased to room temperature, a clear difference between DDM and the novel amphipathic molecules in maintaining receptor activity was observed. β2ARs solubilized in all novel amphipathic compounds showed two to three times higher activity than proteins solubilized in DDM. Among the novel amphipathic compounds, PSE-C11 was the best, followed by PSE-C13 and PSA-C11.

<9-4> Evaluation of Ability of TPSs to Stabilize Structure of β2ARs Membrane Proteins

The ability of TPSs (TPS-As, TPS-Es and TPS-ELs) to stabilize the structures of human β2 adrenergic receptors (β2ARs) and G protein-coupled receptors (GPCRs) was measured. That is, DDM-purified receptors were diluted with a buffer solution containing only each of the TPSs without cholesteryl hemisuccinate (CHS) or a buffer solution containing CHS and DDM. The final concentration of the compounds was CMC+0.2wt %, and the binding activity of the receptors to ligands was evaluated by measuring the degree of binding of the receptors to [³H]-dihydroalprenolol ([³H]-DHA).

Specifically, radioactive ligand binding assay was performed as follows. β2ARs were purified with 0.1 wt % DDM (D. M. Rosenbaum et al., Science, 2007, 318, 1266-1273.) and was concentrated to about 10 mg/mL (approximately 200 μM). β2ARs purified with DDM were used to prepare a master binding mixture containing 10 nM [³H]-dihydroalprenolol (DHA), which was dissolved in 0.2% amphipathic compounds (DDM or TPSs) and supplemented with 0.5 mg/mL BSA. The activity of the receptors purified with the amphipathic compounds was monitored at regular intervals over a 3- to 5-day incubation period. The receptors purified with DDM or TPSs were incubated with 10 nM [³H]-DHA at room temperature for 30 minutes. The mixture was loaded onto a G-50 column, a solution passed through the column was collected in 1 mL of a binding buffer (100 mM NaCl and 20 mM HEPES (pH 7.5) supplemented with 0.5 mg/mL BSA and 20×CMC of each amphipathic compound), and 15 mL of a scintillation fluid was added to the solution. Receptor-bound [³H]-DHA was measured using a scintillation counter (Beckman). The binding degree of the receptors to [³H]-DHA was represented by a column graph.

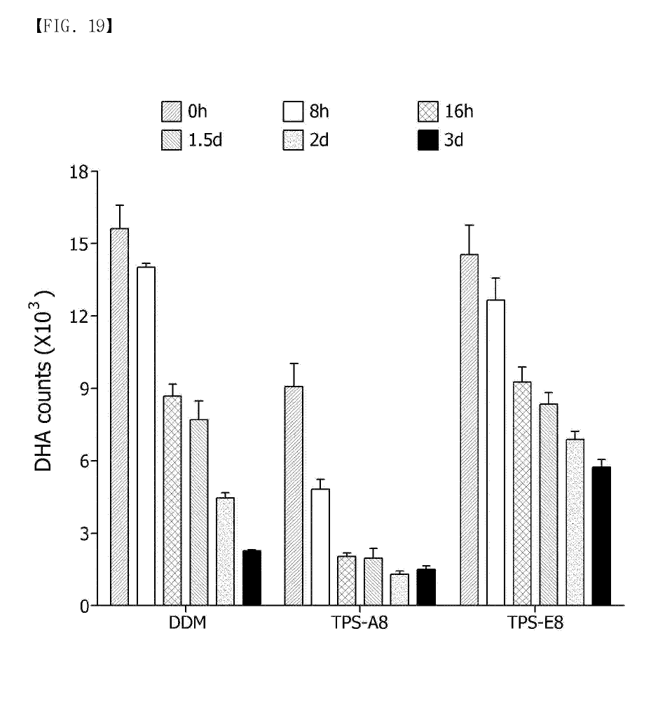

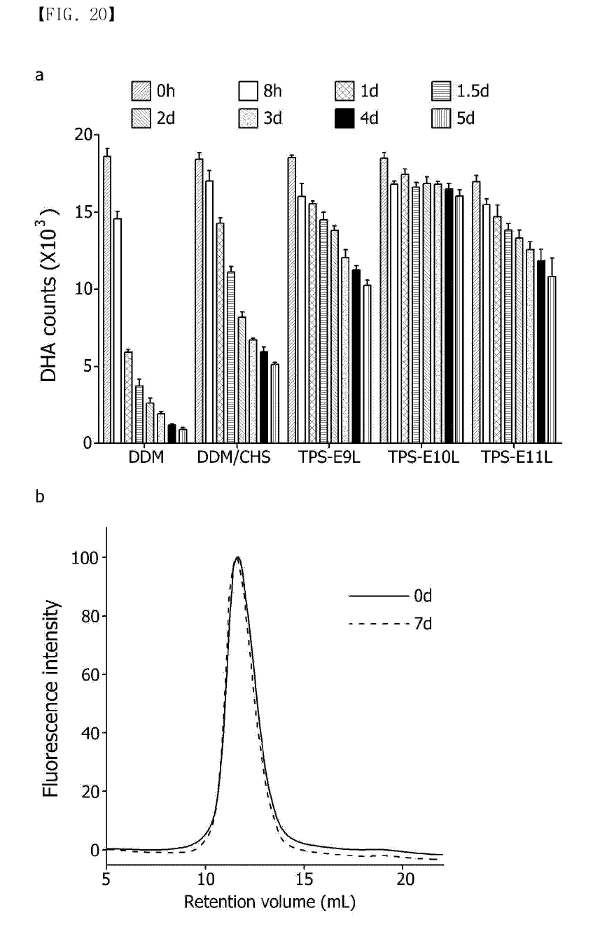

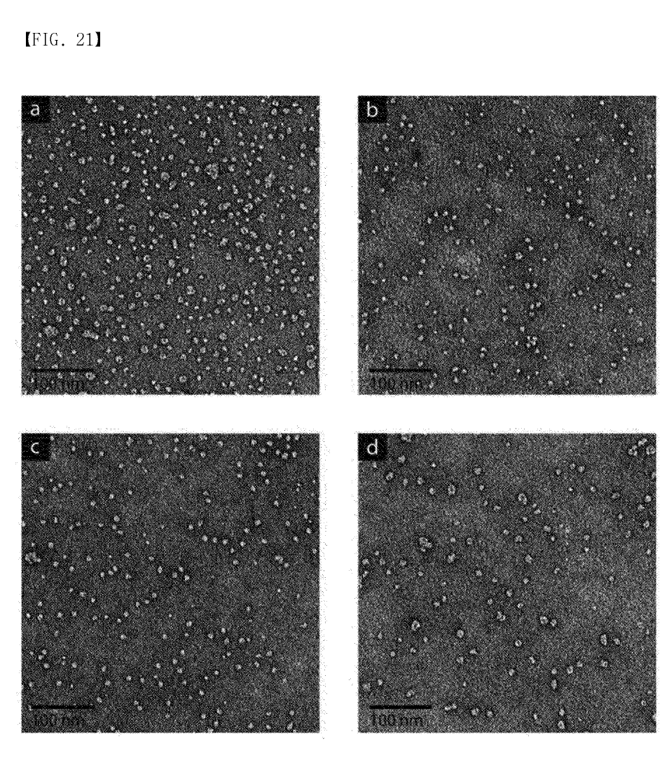

As a result, in the case of TPS-As/Es, the receptor stabilizing ability of TPS-Es was substantially better than that of TPS-As having the same alkyl chain length (e.g., TPS-E7 vs. TPS-A7), and TPS-E8 among TPS-Es was the most excellent (FIG. 18a). In addition, the effect of this compound was significantly increased with increasing an alkyl chain length. In TPS-ELs, all receptors solubilized in each of TPS-E8L, TPS-E9L, TPS-E10L and TPS-E11L exhibited similar activity to receptors solubilized in DDM (FIG. 18b). Based on the results of initial receptor activity, four excellent candidates (TPS-E8, TPS-E9L, TPS-E10L and TPS-E11L) were selected for further evaluation and the binding activity of the receptors to ligands was measured at regular intervals for 3 or 5 days at room temperature. As a result, DDM-solubilized receptors exhibited high initial activity, but the activity was rapidly lost over time. However, when the long-term activity of receptors solubilized in each of TPS-E8, TPS-E9L, TPS-E10L and TPS-E11L was measured, the ability of these compounds to maintain the long-term activity was significantly better than that of DDM and was also superior to that of DDM containing CHS. It is known that CHS binds to the surface of a receptor and improves the stability of the receptor. In particular, TPS-E10L was excellent enough to maintain initial receptor activity up to 90% even after 5 days of incubation (FIGS. 19 and 20a).

<9-5> Size Measurement of β2AR Complexes Formed by PSAs and PSEs

Size exclusion chromatography (SEC) was performed to measure the size of β2AR complexes formed by PSAs and PSEs.

Specifically, size exclusion chromatography (SEC) was performed as follows. β2ARs solubilized in 0.1 wt % DDM was loaded onto a M1 Flag column in the presence of 2 mM CaCl₂, and the column was washed with a DDM/PSA/PSE buffer (20 mM HEPES (pH 7.5), 100 mM NaCl, 0.2 wt % amphipathic compound). Receptors were eluted with 20×CMC DDM/PSA/PSE with 5 mM EDTA and 0.2 mg/mL free Flag peptides. The eluents were applied to a Superdex-200 10/300 GL column (GE Healthcare) at a flow rate of 0.5 mL/min, and UV absorbance was measured at 280 nm. A running buffer contained 20 mM HEPES (pH 7.5), 100 mM NaCl, 20×CMC of each amphipathic compound (DDM, PSA-C11, PSE-C11 and PSE-C13).

As shown in FIG. 17b, all amphipathic compounds formed homogeneous complexes (protein-detergent complexes, PDCs) with the receptors, and these PDCs were distinctly smaller than PDCs formed by DDM.

<9-6> Electron Microscopy (EM) Analysis of β2ARs Solubilized in PSAs and PSEs

Electron microscopy (EM) analysis was performed on β2ARs solubilized in PSAs and PSEs.

Specifically, electron microscopy (EM) analysis was performed as follows. Samples were prepared using a conventional negative staining protocol (see Peisley, A. et al., G Protein-Coupled Receptors in Drug Discovery: Methods and Protocols, Methods in Molecular Biology, 1335, 29-38 (2015)). Briefly, 3 μL of β2ARs purified in DDM or the novel amphipathic compounds (PSA-C11, PSE-C11 and PSE-C13) was added into a glow-discharged carbon-coated grid by pipetting and stained with 1% (w/v) uranyl formate. Imaging was performed by operating a Morgagni 268 (D) transmission electron microscope (FEI Company) at 100 kV at room temperature. Images were recorded with an Orius SC200W CCD camera (Gatan Inc.) at a 30,416×magnification.

FIG. 21 shows the negative staining EM images of β2ARs purified with DDM (FIG. 21a), PSA-C11 (FIG. 21b), PSE-C11 (FIG. 21c), or PSE-C13 (FIG. 21d). In the images of the receptors dissolved in DDM, many agglutinated proteins were observed. Agglutinated proteins were also observed in the receptors solubilized in PSE-C13, but the amount of the agglutinated proteins was small. In the case of PSA-C11 and PSE-C11, the agglutinated proteins were hardly observed. Therefore, it was confirmed that PSAs and PSEs may be used to study the structures of membrane proteins using electron microscopy.

<9-7> Evaluation of Ability of PSE-C11 to Extract and Solubilize β2ARs

The best, PSE-C11, among tested PSAs/PSEs was used to extract β2ARs directly from the cell membranes. Receptors were treated with 1.0 wt % PSE-C11 or DDM, and the activity of the receptors solubilized in PSE-C11 or DDM was measured using a radiolabeled ligand, [³H]-DHA.

Specifically, experiments to extract and solubilize receptors from the cell membranes were performed as follows. 10 mL of a PSE-C11 amphipathic compound buffer (20 mM HEPES (pH 7.5), 100 mM NaCl, 1.0 wt % PSE-C11) was added to 1 g of an insect cell (Sf9) pellet expressing β2ARs. The mixture was stirred for solubilization for 1 hour. After performing centrifugation at 12,000 g for 20 minutes, a supernatant was collected and loaded onto a M1 Flag column in the presence of 2 mM CaCl₂. The column was washed with a PSE-C11 buffer (20 mM HEPES (pH 7.5), 100 mM NaCl, 0.2 wt % amphipathic compound). Receptors were eluted with 20×CMC PSE-C11, 5 mM EDTA and 0.2 mg/mL free Flag peptides. To measure the activity of the receptors solubilized and purified by DDM or PSE-C11, 0.2 pmol of β2ARs was incubated with 10 mM [³H]-DHA and each amphipathic compound at room temperature for 30 minutes, and the activity of β2ARs was measured. The following procedure was performed in the same manner as described in Example 9-3. Each measurement was performed three times. The receptors solubilized in 20×CMC PSE-C11 or 20×CMC DDM were also applied to SEC under a buffer (20 mM HEPES (pH 7.5), 100 mM NaCl) not containing an amphipathic compound (PSE-C11 or DDM).

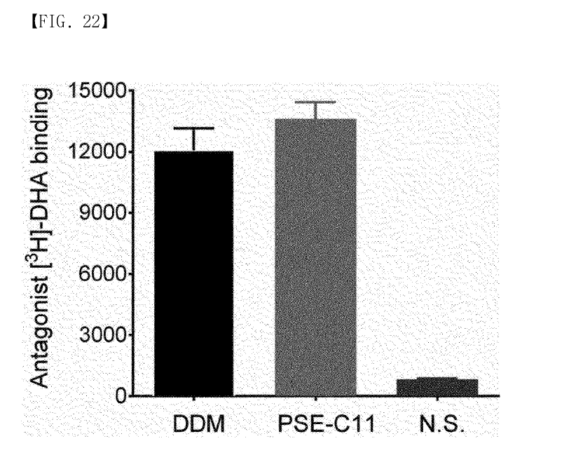

FIG. 22 shows the results of a solubilization test. The receptors extracted by PSE-C11 exhibited higher activity than proteins extracted by DDM. From this result, it was confirmed that this amphipathic compound could be a substitute for DDM in GPCR solubilization.

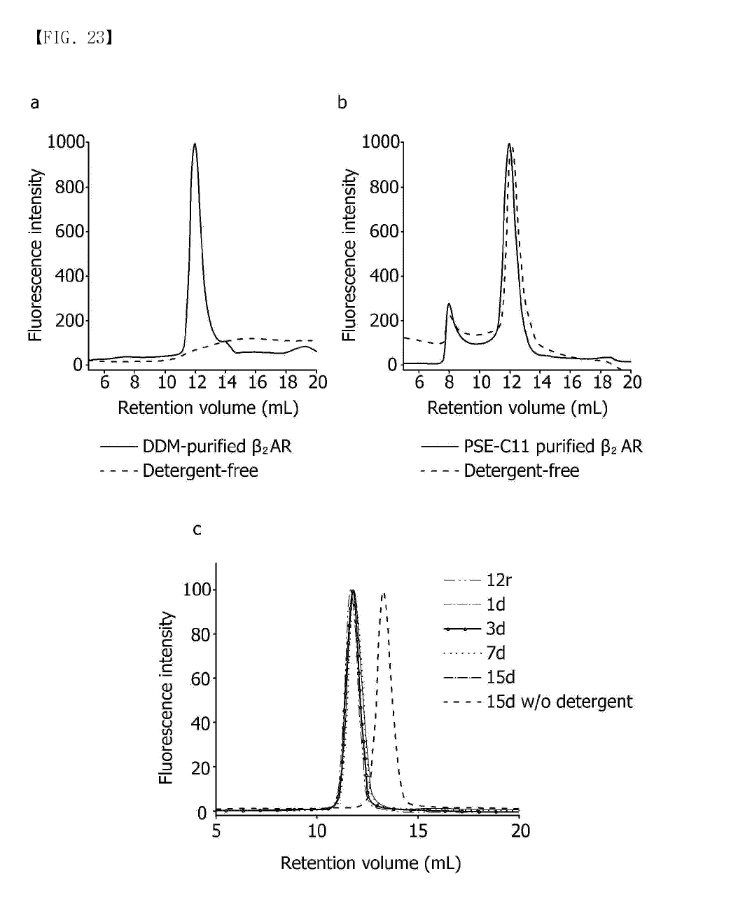

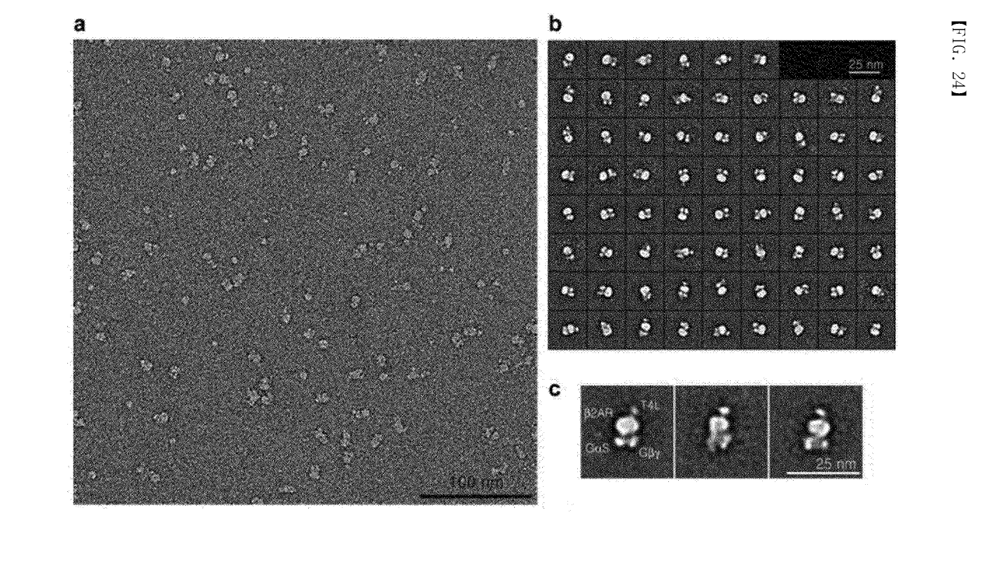

FIG. 23 shows the results of SEC experiments using a buffer not containing amphipathic compounds after extracting the receptors from the cell membranes using an amphipathic compound (PSE-C11 or DDM). In this experiment, no peak corresponding to the proteins was observed in the case of the receptors solubilized in DDM, indicating that the receptor purified by DDM was completely denatured/aggregated during this experimental procedure (FIG. 23a). On the other hand, the receptors purified by PSE-C11 exhibited a distinct monodisperse peak, even though using a buffer not containing amphipathic compounds (FIG. 23b). This peak was nearly identical to that obtained from proteins analyzed using a buffer containing amphipathic compounds. Therefore, this indicates that the stability of the receptors is well maintained in this amphipathic molecule. This is presumably due to the strong binding affinity of this amphipathic molecule and the slow separation rate of PSE-C 11 from the receptors. These properties of PSE-C11 may be used to remove excess micelles from PSE-C11-protein complexes, which is important for the structure studies of various membrane proteins.

<9-8> Evaluation of PSE-C11 or TPS-E10L in T4L-β₂AR-Gₛ or β₂AR-Gₛ Complexes

The ability of PSE-C11 or TPS-E10L to purify and stabilize T4L-β₂AR-Gₛ or β₂AR-Gₛ complexes was measured using electron microscopy (EM) analysis.

Specifically, measurement of the ability of PSE-C11 for TPS-E10L to purify and stabilize T4L-β₂AR-Gₛ or β₂AR-Gₛ complexes was performed as follows. 100 μM T4L-β₂AR solubilized in 0.1 wt % DDM was mixed with 120 μM Gₛ heterotrimers for 30 minutes. 0.5 units of apyrase (NEB) and 2 mM MgCl₂ were added to the mixture, followed by a 1-hour incubation to form complexes. 1 wt % PSE-C11 or TPS-E10L was respectively added to the mixture to achieve a final concentration of 0.8%, and incubated for 30 minutes to allow DDM to be exchanged with PSE-C11 or TPS-E10L. Protein solutions were loaded onto a M1 Flag column, and the column was washed with sequential buffers, which resulted in complete exchange with a 0.5% PSE-C11 or TPS-E10L buffer in a 0.1% DDM buffer at different molar ratios. Finally, proteins were eluted with a 0.05% (100×CMC) PSE-C11 or TPS-E10L buffer. Gel filtration was performed using a running buffer (20 mM HEPES (pH 7.5), 100 mM NaCl, 0.005% PSE-C11 or TPS-E10L, 1 μM BI, 100 μM TCEP) to purify T4L-β₂AR-Gₛ complexes. To measure the stability of β₂AR-Gₛ complexes present in PSE-C11 or TPS-E10L, analytical gel filtration was performed with running buffers of the same formulation at 12 hours, 1 day, 3 days, 7 days, and 15 days. After a 15 day-incubation, analytical gel filtration was performed using an amphiphilic compound pre-buffer having the same formulation as above without PSE-C11 or TPS-E10L.

In addition, negative staining EM analysis for T4L-β₂AR-Gₛ or β₂AR-Gₛ complexes present in PSE-C11 was performed as follows. Samples containing T4L-β₂AR-Gₛ or β₂AR-Gₛ were prepared to perform electron microscopy analysis using a conventional negative staining protocol (see Peisley, A. et al., G Protein-Coupled Receptors in Drug Discovery: Methods and Protocols, Methods in Molecular Biology, 1335, 29-38 (2015)), and imaging was performed by operating a Tecnai T12 electron microscope at 120 kV at room temperature. Images were recorded with a Gatan US4000 CCD camera at a 71,138×magnification with a defocus value of ˜1.5 μM.

As shown in FIGS. 20b and 23c, the remarkable effect of PSE-C11 or TPS-E10L on the stability of β₂AR-Gₛ complexes was confirmed. That is, after the compounds were incubated for 15 and 17 days, no separation of the complexes was observed at all. It has been reported in the prior art that complex separation was observed even after 2 days of incubation of the T4L-β₂AR-Gₛ complexes purified by DDM at 4° C. Furthermore, even when a buffer without the amphipathic compound was used as an eluent, the complexes solubilized in PSE-C11 were observed to be completely stable.

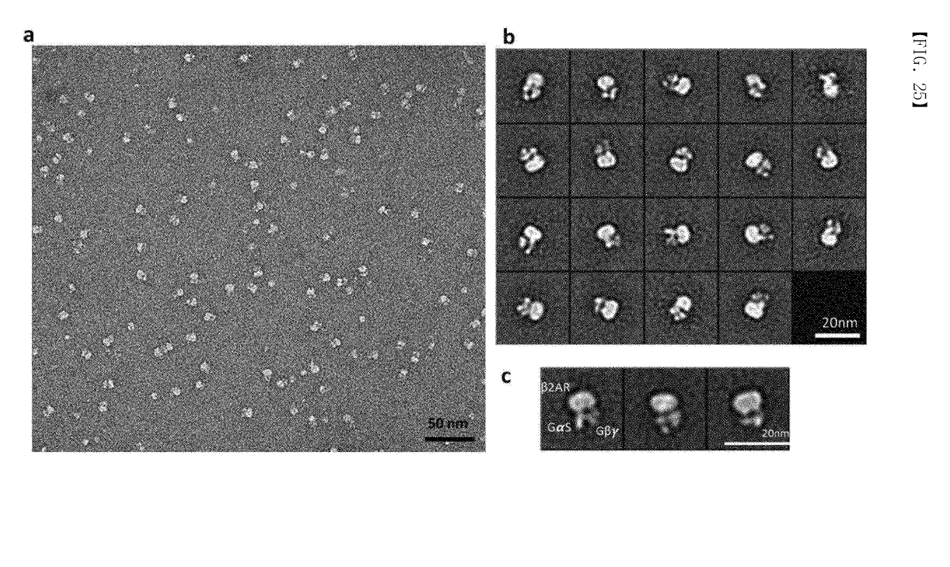

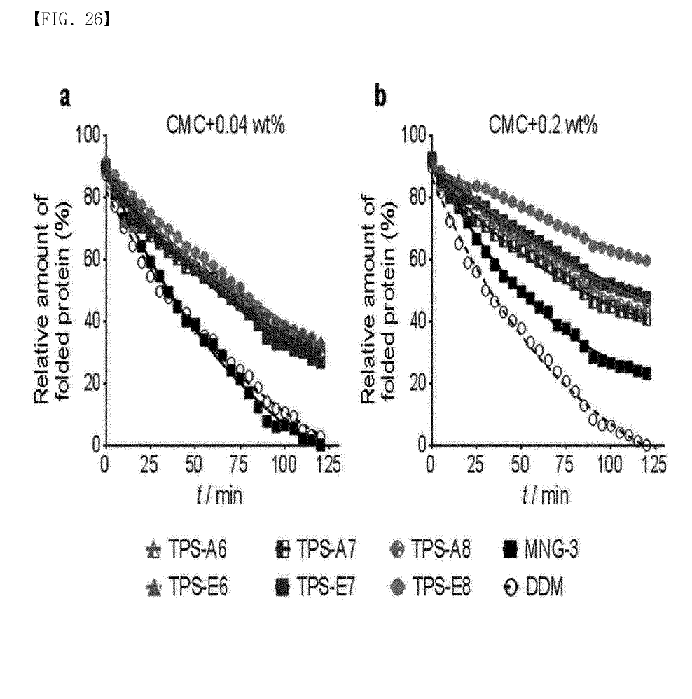

FIGS. 24 and 25 show EM analysis results of complexes purified by PSE-C11 and TPS-E10L. Negative staining EM images showed high monodispersibility without aggregation or denaturation. When image analysis was performed using 2D classification and particle averaging methods, each domain (β₂AR, G_{αS} and G_{βη}) constituting these complexes was clearly distinguished. The structures of the observed complexes are exactly the same as the structures of the complexes dissolved by MNG-3. Thus, these results suggest that PSE-C11 or TPS-E10L may be possibly used for imaging and crystallization of easily separable membrane protein complexes.

### EXAMPLE 10

### Evaluation of Ability of TPSs (TPS-As, TPS-Es and TPS-ELs) to Stabilize Structure of UapA Membrane Protein

The ability of TPSs (TPS-As, TPS-Es and TPS-ELs) to stabilize the structure of uric acid-xanthine/H+ symporter (UapA) isolated from Aspergillus nidulans was measured. The structural stability of UapA was evaluated using a sulfhydryl-specific fluorophore, N-[4-(7-diethylamino-4-methyl-3-coumarinyl)phenyl]maleimide (CPM).

Specifically, UapAG411V₁₋₁₁ (hereinafter, referred to as ‘UapA’) was expressed in a Saccharomyces cerevisiae FGY217 strain as a GFP fusion protein, and was isolated using a sample buffer (20 mM Tris (pH 7.5), 150 mM NaCl, 0.03% DDM, 1 mM xanthine). The transporter was concentrated to about 10 mg/mL using a 100 kDa molecular weight cut-off filter (Millipore). The transporter was diluted with a CMC+0.04 wt % or CMC+0.2 wt % buffer at a ratio of 1:150 in a Greiner 96-well plate containing one of TPS-As (TPS-A6, TPS-A7 or TPS-A8), TPS-Es (TPS-E6, TPS-E7 or TPS-E8), TPS-ELs (TPS-E8L, TPS-E9L, TPS-E10L or TPS-E11L), MNG-3 or DDM (control group). A CPM dye (Invitrogen) stored in DMSO (Sigma) was diluted with a dye buffer (20 mM Tris (pH 7.5), 150 mM NaCl, 0.03% DDM, 5 mM EDTA). 3 μL of the diluted dye solution was added to each protein sample. The reaction mixture was incubated at 40° C. for 120 minutes. During incubation, fluorescence emission intensity was monitored using a microplate spectrophotometer set at excitation and emission wavelengths of 387 and 463 nm, respectively. The maximum value of fluorescence intensity was used to calculate the relative percentage of folded transporters during the incubation period. The relative amount of folded transporters was plotted over time using GraphPad Prism.

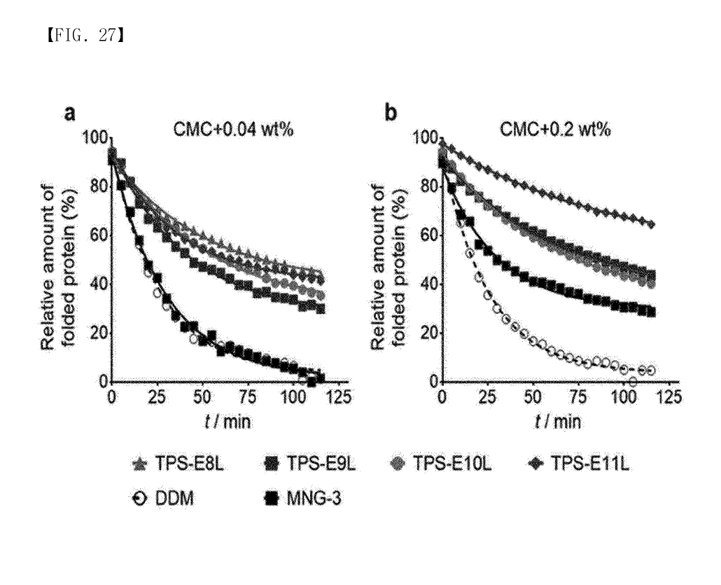

As shown in FIGS. 26 and 27, compared with DDM, all TPSs (TPS-As, TPS-Es and TPS-ELs) were excellent in maintaining a folding state of UapA proteins at all tested concentrations. In particular, TPS-E8 was the most excellent among TPS-As/Es (FIG. 26), and TPS-E11L among TPS-ELs showed the best effect at a high concentration (CMC+0.2 wt %) (FIG. 27).

Based on these results, it was confirmed that TPSs (TPS-As, TPS-Es and TPS-ELs) were excellent in maintaining, in an aqueous solution, structural stability of UapA extracted from the cell membranes. Thus, TPSs may be effectively used to stabilize membrane proteins.

When amphipathic compounds having a branched penta-saccharide hydrophilic group according to the present invention were used, compared to the existing compounds, membrane proteins or the complexes thereof can be stably stored in an aqueous solution for a long period of time. In addition, the amphipathic compounds of the present invention are excellent in solubilizing membrane proteins and thus can be used to analyze the functions and structures of membrane proteins or the complexes thereof.

Structural and functional analysis of membrane proteins is one of the areas of greatest interest in current biology and chemistry, and more than half of the new drugs currently being developed target membrane proteins. Accordingly, the compound of the present invention can be applied to protein structural studies that are closely related to the development of new drugs.

Specifically, since the compounds according to embodiments of the present invention has a high-density hydrophilic group composed of five glucose units, the compounds can have an excellent effect on the crystallization of membrane proteins. In addition, since the hydrophilic group is a hydrophilic group having a novel structure used in amphipathic compounds, the hydrophilic group can be applied to the development of the structures of various amphipathic molecules.

In addition, the compounds according to embodiments of the present invention can be synthesized from readily available starting materials in a relatively simple manner, allowing for mass production of the compounds for membrane protein studies.

What is claimed is:

1. A compound represented by Formula 1 below: 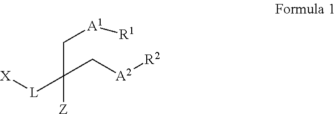
wherein L represents a substituted or unsubstituted C₁-C₁₀ alkylene group, or a direct bond;
A¹ and A² represent methylene groups or oxygen atoms;
each of R¹ and R² independently represents a substituted or unsubstituted C₃-C₂₀ alkyl group, a substituted or unsubstituted C₃-C₂₀ cycloalkyl group, or a substituted or unsubstituted C₃-C₂₀ aryl group;
X represents a glucose-centered branched penta-saccharide linked by oxygen; and
Z represents a hydrogen atom or —CH₂-A³-R³, wherein A³ represents a methylene group or an oxygen atom, and R³ independently represents a substituted or unsubstituted C₃-C₂₀ alkyl group, a substituted or unsubstituted C₃-C₂₀ cycloalkyl group, or a substituted or unsubstituted C₃-C₂₀ aryl group.

2. The compound according to claim 1, wherein L represents a methylene group; A¹ and A² represent methylene groups; each of R¹ and R² independently represents a substituted or unsubstituted C₃-C₂₀ alkyl group, a substituted or unsubstituted C₃-C₂₀ cycloalkyl group, or a substituted or unsubstituted C₃-C₂₀ aryl group; and Z represents a hydrogen atom.

3. The compound according to claim 1, wherein L represents a methylene group; A¹ and A² represent methylene groups; R¹ and R² represent substituted or unsubstituted C₅-C₁₅ alkyl groups; R₁ and R₂ are the same; and Z represents a hydrogen atom.

4. The compound according to claim 1, wherein L represents a direct bond; A¹ and A² represent oxygen atoms; each of R¹ and R² independently represents a substituted or unsubstituted C₃-C₂₀ alkyl group, a substituted or unsubstituted C₃-C₂₀ cycloalkyl group, or a substituted or unsubstituted C₃-C₂₀ aryl group; and Z represents a hydrogen atom.

5. The compound according to claim 1, wherein L represents a direct bond; A¹ and A² represent oxygen atoms; R¹ and R² represent substituted or unsubstituted C₅-C₁₅ alkyl groups; R¹ and R² are the same; and Z represents a hydrogen atom.

6. The compound according to claim 1, wherein L represents a methylene group; and Z represents —CH₂-A³-R³, wherein one or more of A¹ to A³ represent oxygen atoms and the other(s) represent(s) methylene groups; each of R¹ to R³ independently represents a substituted or unsubstituted C₃-C₂₀ alkyl group, a substituted or unsubstituted C₃-C₂₀ cycloalkyl group, or a substituted or unsubstituted C₃-C₂₀ aryl group; and R¹ to R³ are the same.

7. The compound according to claim 1, wherein L represents a methylene group; and Z represents —CH₂-A³-R³, wherein one or more of A¹ to A³ represent oxygen atoms and the other(s) represent(s) methylene groups; each of R¹ to R³ independently represents a substituted or unsubstituted C₃-C₂₀ alkyl group; and R¹ to R³ are the same.

8. The compound according to claim 1, wherein the compound is represented by one of Formulas 2 to 18 below:

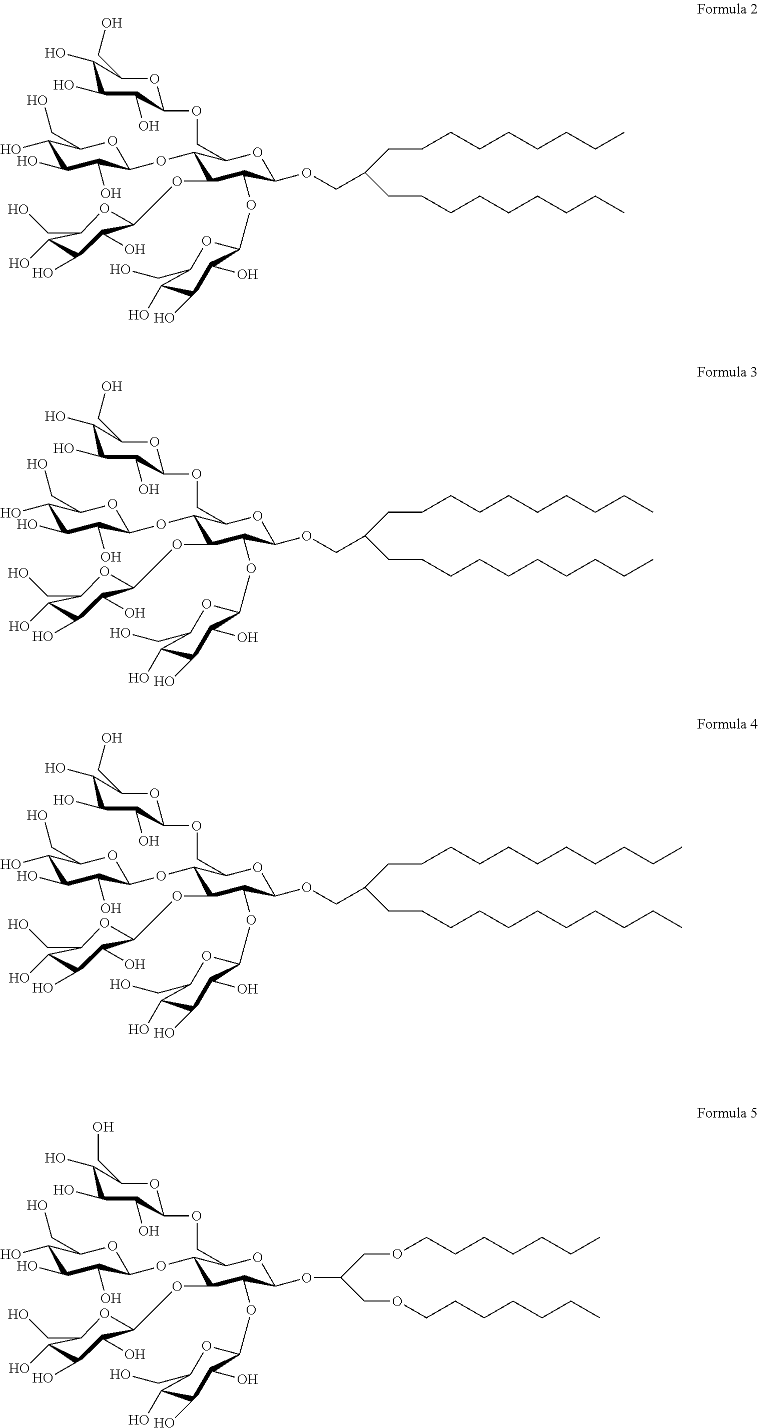

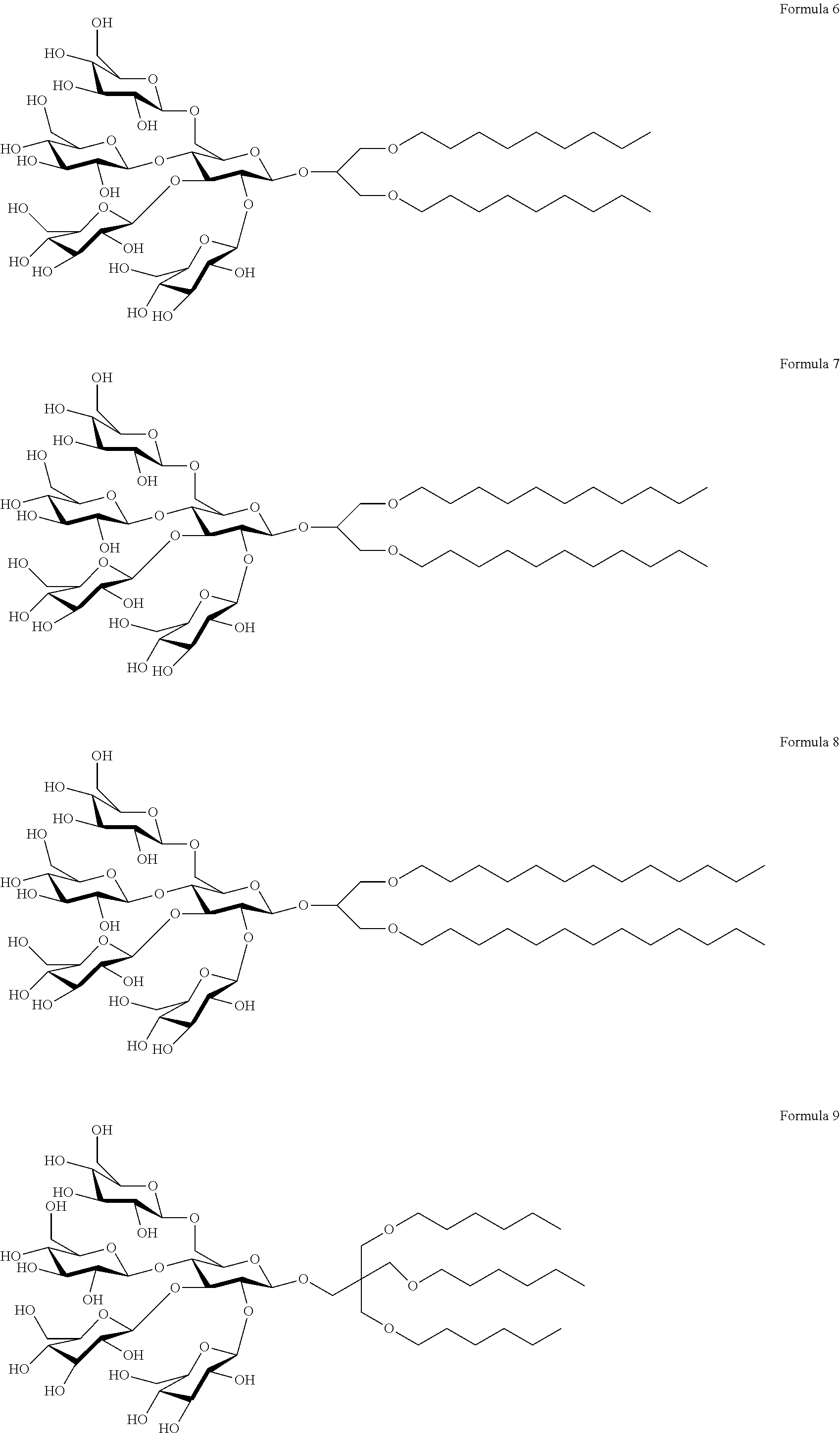

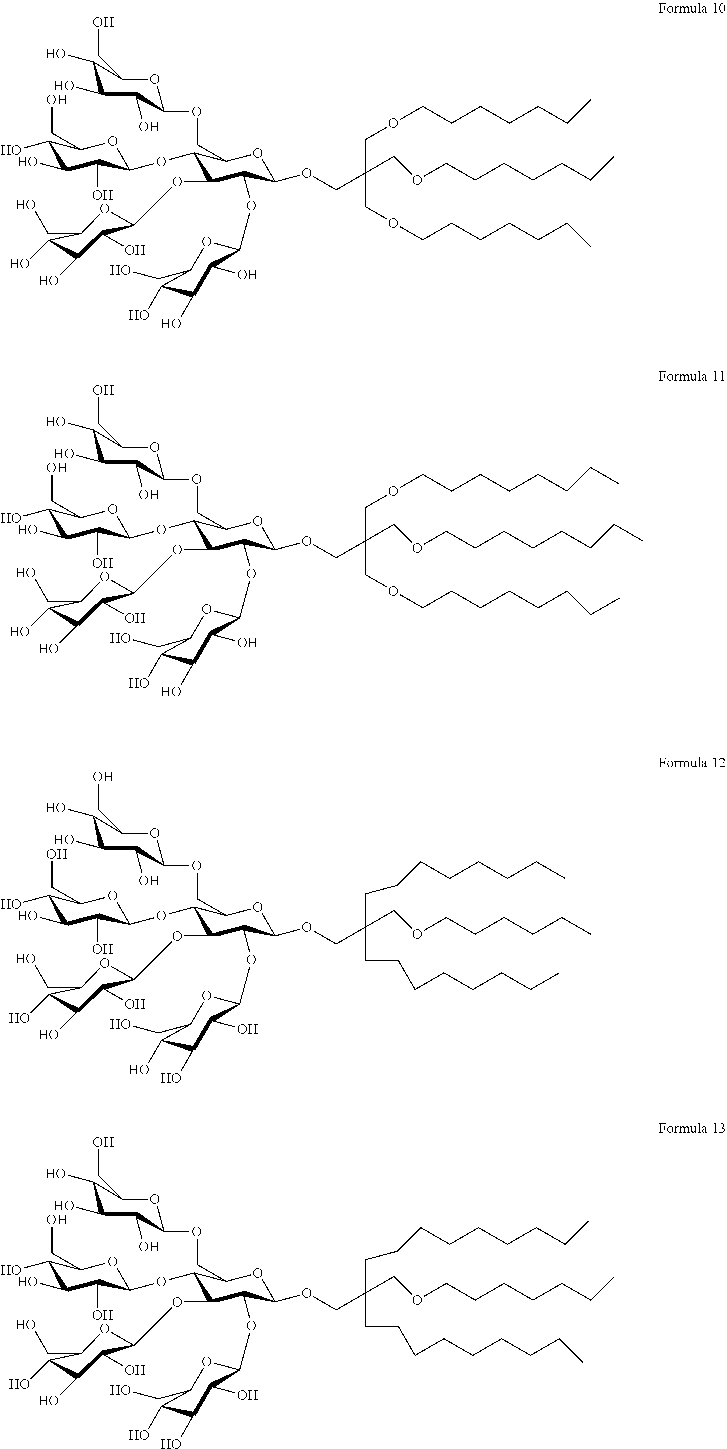

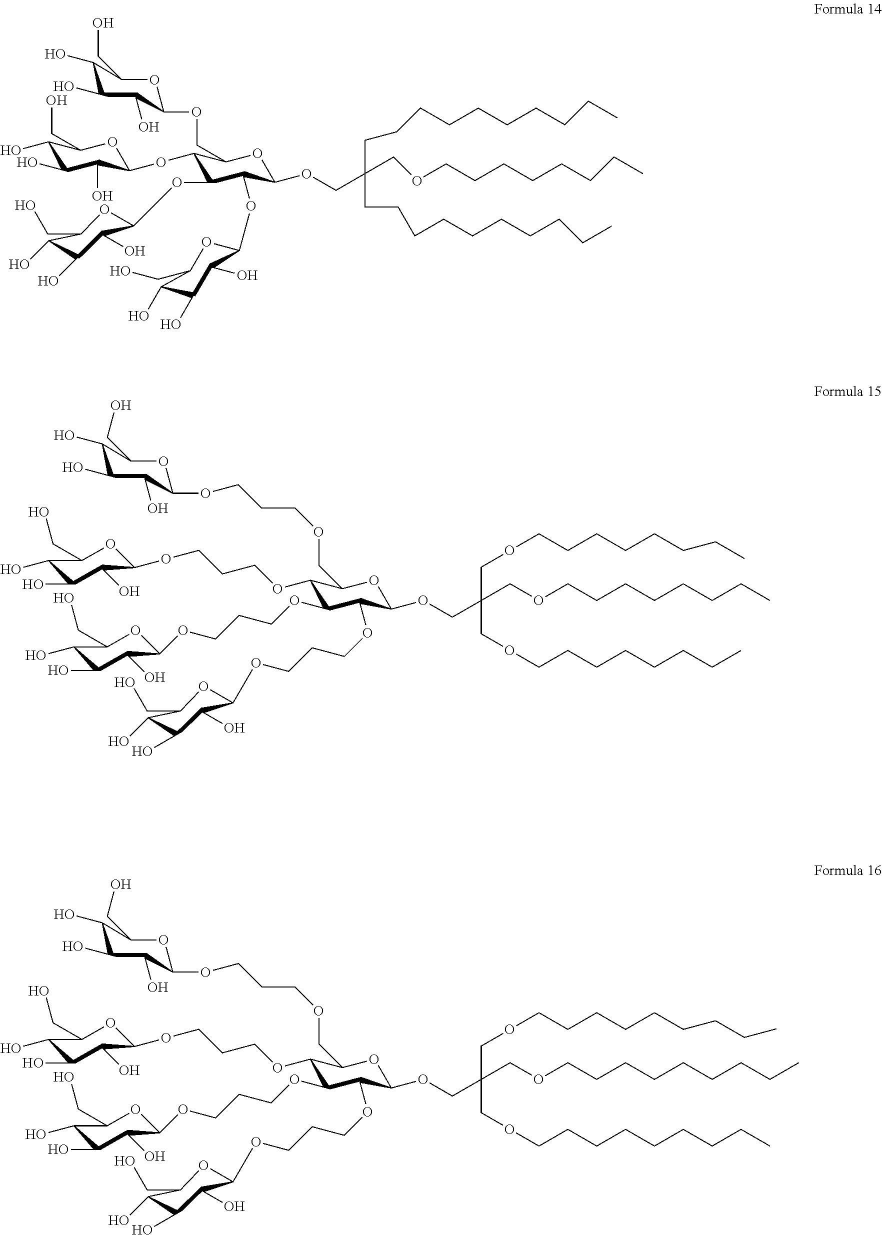

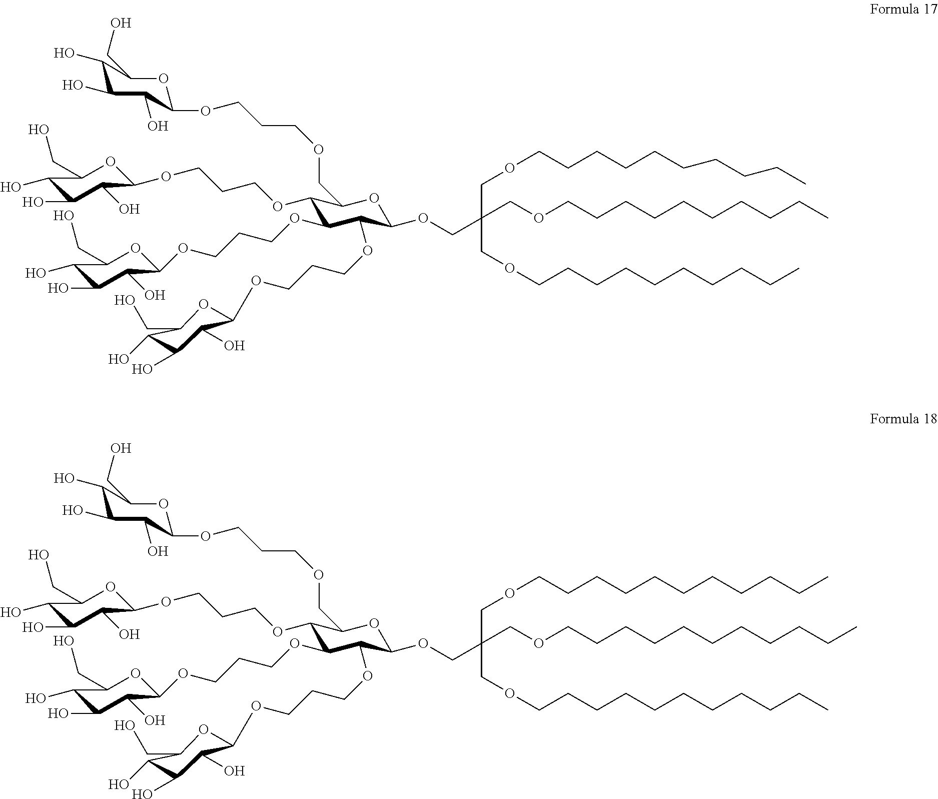

9. The compound according to claim 1, wherein the compound is an amphipathic molecule for extracting, solubilizing, stabilizing, crystallizing or analyzing membrane proteins.

10. The compound according to claim 1, wherein the compound has a critical micelle concentration (CMC) of 0.0001 to 0.1 mM in an aqueous solution.

11. A composition for extracting, solubilizing, stabilizing, crystallizing or analyzing membrane proteins, comprising the compound according to claim 1.

12. The composition according to claim 11, wherein the composition is prepared in a form of micelles, liposomes, emulsions or nanoparticles.

13. A method of preparing a compound represented by Formula 1 below, the method comprising:
preparing dialkylated diethylmalonate by adding a 1-iodoalkane to diethyl malonate;
preparing a dialkylated mono-ol by adding LiCl, DMSO and H₂O to the prepared dialkylated diethylmalonate, heating the mixture to a temperature of 150 to 200° C., and adding LiAlH₄ and THF to the mixture;
introducing a protecting group-attached glucose by performing a glycosylation reaction on the prepared dialkylated mono-ol;
removing an O-benzoyl group by performing a deprotection reaction on the product prepared in the introducing;
attaching four glucose units with attached protecting groups by performing a glycosylation reaction on the product prepared in the removing to introduce a penta-saccharide hydrophilic group; and
removing an O-benzoyl group by performing a deprotection reaction on the product prepared in the attaching, 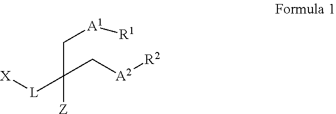
wherein L represents a methylene group; A¹ and A² represent methylene groups; each of R¹ and R² independently represents a substituted or unsubstituted C₃-C₂₀ alkyl group, a substituted or unsubstituted C₃-C₂₀ cycloalkyl group, or a substituted or unsubstituted C₃-C₂₀ aryl group; Z represents a hydrogen atom; and X represents a glucose-centered branched penta-saccharide.

14. A method of preparing a compound represented by Formula 1 below, the method comprising:
preparing an alcohol derivative by adding NaOH and an alcohol to epichlorohydrin;
introducing a protecting group-attached glucose by performing a glycosylation reaction on the prepared alcohol derivative;
removing an O-benzoyl group by performing a deprotection reaction on the product prepared in the introducing;
attaching four glucose units with attached protecting groups by performing a glycosylation reaction on the product prepared in the removing to introduce a penta-saccharide hydrophilic group: and
removing an O-benzoyl group by performing a deprotection reaction on the product prepared in the attaching, 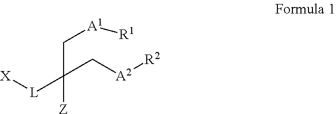
wherein L represents a direct bond; A¹ and A² represent oxygen atoms; each of R¹ and R² independently represents a substituted or unsubstituted C₃-C₂₀ alkyl group, a substituted or unsubstituted C₃-C₂₀ cycloalkyl group, or a substituted or unsubstituted C₃-C₂₀ aryl group; Z represents a hydrogen atom; and X represents a glucose-centered branched penta-saccharide.

15. A method of preparing a compound represented by Formula 1 below, comprising:
preparing dialkylated diethylmalonate by adding a 1-iodoalkane to diethyl malonate;
preparing a dialkylated diol by adding LiAlH₄ and THF to the prepared dialkylated diethylmalonate;
adding an alkyl chain by adding a 1-bromoalkane to the prepared dialkylated diol;
introducing a protecting group-attached glucose by performing a glycosylation reaction on the product prepared in the adding;
removing an O-benzoyl group by performing a deprotection reaction on the product prepared in the introducing;
attaching four glucose units with attached protecting groups by performing a glycosylation reaction on the product prepared in the removing to introduce a penta-saccharide hydrophilic group; and
removing an O-benzoyl group by performing a deprotection reaction on the product prepared in the attaching, 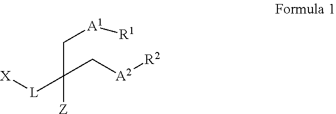
wherein L represents a methylene group; Z represents —CH₂-A³-R³, wherein one of A¹ to A³ represents an oxygen atom and the others represent methylene groups; each of R¹ to R³ independently represents a substituted or unsubstituted C₃-C₂₀ alkyl group; and R¹ to R³ are the same; and X represents a glucose-centered branched penta-saccharide.

16. A method of preparing a compound represented by Formula 1 below, the method comprising:
synthesizing a dialkylated diol using 5,5-bis-bromomethyl-2,2-dimethyl-[1,3]dioxane as a starting material;
adding an alkyl chain by adding a 1-bromoalkane to the product prepared in the synthesizing;
introducing a protecting group-attached glucose by performing a glycosylation reaction on the product prepared in the adding;
removing an O-benzoyl group by performing a deprotection reaction on the product prepared in the introducing;
attaching four glucose units with attached protecting groups by performing a glycosylation reaction on the product prepared in the removing to introduce a penta-saccharide hydrophilic group; and
removing an O-benzoyl group by performing a deprotection reaction on the product prepared in the attaching, 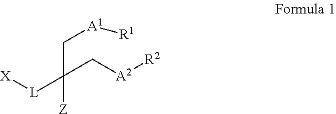
wherein L represents a methylene group; Z represents —CH₂-A³-R³, wherein A¹ to A³ represent oxygen atoms; each of R¹ to R³ independently represents a substituted or unsubstituted C₃-C₂₀ alkyl group; and R¹ to R³ are the same; and X represents a glucose-centered branched penta-saccharide.

17. A method of extracting, solubilizing, stabilizing, crystallizing or analyzing membrane protein, the method comprising treating membrane proteins with a compound represented by Formula 1 below in an aqueous solution: 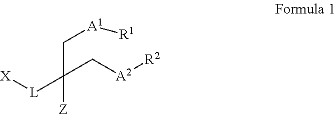
wherein L represents a substituted or unsubstituted C₁-C₁₀ alkylene group, or a direct bond;
each of A¹ and A² represents a methylene group or an oxygen atom;
each of R¹ and R² independently represents a substituted or unsubstituted C₃-C₂₀ alkyl group, a substituted or unsubstituted C₃-C₂₀ cycloalkyl group, or a substituted or unsubstituted C₃-C₂₀ aryl group;
X represents a glucose-centered branched penta-saccharide linked by oxygen; and
Z represents a hydrogen atom or —CH₂-A³-R³, wherein A³ represents a methylene group or an oxygen atom, and R³ independently represents a substituted or unsubstituted C₃-C₂₀ alkyl group, a substituted or unsubstituted C₃-C₂₀ cycloalkyl group, or a substituted or unsubstituted C₃-C₂₀ aryl group.

18. The method according to claim 17, wherein L represents a methylene group; A¹ and A² represent methylene groups; each of R¹ and R² independently represents a substituted or unsubstituted C₃-C₂₀ alkyl group, a substituted or unsubstituted C₃-C₂₀ cycloalkyl group, or a substituted or unsubstituted C₃-C₂₀ aryl group; and Z represents a hydrogen atom.

19. The method according to claim 17, wherein L represents a direct bond; A¹ and A² represent oxygen atoms; each of R¹ and R² independently represents a substituted or unsubstituted C₃-C₂₀ alkyl group, a substituted or unsubstituted C₃-C₂₀ cycloalkyl group, or a substituted or unsubstituted C₃-C₂₀ aryl group; and Z represents a hydrogen atom.

20. The method according to claim 17, wherein L represents a methylene group; and Z represents —CH₂-A³-R³, wherein one or more of A¹ to A³ represent oxygen atoms and the other(s) represent(s) methylene groups; each of R¹ to R³ independently represents a substituted or unsubstituted C₃-C₂₀ alkyl group, a substituted or unsubstituted C₃-C₂₀ cycloalkyl group, or a substituted or unsubstituted C₃-C₂₀ aryl group; and R¹ to R³ are the same.

21. The method according to claim 17, wherein the membrane proteins are boron transporter (BOR1), leucine transporter (LeuT), melibiose permease (MelB), human β2 adrenergic receptors (β2ARs), uric acid-xanthine/H+ symporter (UapA), or a combination of two or more thereof.
